(19) United States
(12) Patent Application Publication
(10) Pub. No.: US 2026/0315535 A1
(43) Pub. Date: Oct. 15, 2026

(54) TREATMENT OF AUTOIMMUNE DISORDERS WITH NK CELLS

(71) Applicant: Artiva Biotherapeutics, Inc., San Diego, CA (US)

(72) Inventors: Peter Flynn, San Diego, CA (US); Thorsten Graef, San Diego, CA (US); Fred Aslan, San Diego, CA (US); Heather Karen Raymon, San Diego, CA (US); Lisa Guerrettaz, San Diego, CA (US); Paul Rogers, San Diego, CA (US); Nicholas Veomett, San Diego, CA (US)

(21) Appl. No.: 19/484,095

(22) PCT Filed: May 14, 2024
(86) PCT No.: PCT/US2024/029336

§ 371 Date: Nov. 13, 2025

Related U.S. Application Data
(60) Provisional application No. 63/466,589, filed on May 15, 2023.
(60) Provisional application No. 63/513,154, filed on Jul. 12, 2023.
(60) Provisional application No. 63/603,850, filed on Nov. 29, 2023.
(60) Provisional application No. 63/556,636, filed on Feb. 22, 2024.
(60) Provisional application No. 63/642,218, filed on May 3, 2024.

(51) Int. Cl. A61K 35/17; A61K 31/675; A61K 31/7076; A61K 38/20; A61K 39/00; A61K 39/395; A61P 13/12; A61P 37/06; C12N 5/0783

(52) U.S. Cl. CPC A61K 35/17; A61K 31/675; A61K 31/7076; A61K 38/2013; A61K 39/3955; A61P 13/12; A61P 37/06; C12N 5/0646; A61K 2039/505

(57) ABSTRACT

Provided herein are, among other things, methods for treating a patient suffering from an autoimmune disease.

### CLAIM OF PRIORITY

This application claims the benefit of U.S. Provisional Application Ser. No. 63/466,589, filed on May 15, 2023, U.S. Provisional Application Ser. No. 63/513,154, filed on Jul. 12, 2023, U.S. Provisional Application Ser. No. 63/603,850, filed on Nov. 29, 2023, U.S. Provisional Application Ser. No. 63/556,636, filed on Feb. 22, 2024, and U.S. Provisional Application Ser. No. 63/642,218, filed on May 3, 2024. The entire contents of the foregoing are incorporated herein by reference.

### BACKGROUND

High levels of autoreactive immune cells are associated with autoimmune indications. The incomplete or reduced depletion of pathogenic B cells with antibody alone (ex. Rituximab) in autoimmune patients (for example, lupus) can result in limited efficacy or relapse of disease.

The present invention addresses these and other deficiencies in the art.

### SUMMARY

NK cells are immune cells that can engage tumor cells through a complex array of receptors on their cell surface, as well as through antibody-dependent cellular cytotoxicity (ADCC). To initiate ADCC, NK cells engage with antibodies via the CD16 receptor on their surface. NK cells may have an advantage over other immune cells, such as the T cells used in CAR-T cell therapy and other cell therapies. In an exemplary advantage, NK cells can be used as allogeneic therapies, meaning that NK cells from one donor can be safely used in one or many patients without the requirement for HLA matching, gene editing, or other genetic manipulations. Allogeneic NK cells with anti-tumor activity can be administered safely to patients without many of the risks associated with T cell therapies, such as severe cytokine release syndrome (CRS), and neurological toxicities or graft versus host disease (GvHD).

Allogeneic NK cells may provide an important treatment option for patients with autoimmune disorders, resulting in a deeper and more prolonged depletion of pathogenic B cells than, e.g., antibody alone, resulting in better efficacy and outcomes.

Additionally, cords with preferred characteristics for enhanced clinical activity (e.g., high-affinity CD16 and Killer cell Immunoglobulin-like Receptor (KIR) B-haplotype) can be selected by utilizing a diverse umbilical cord blood bank as a source for NK cells.

The administration of the allogenic NK cells, as described herein, can enhance patients' ADCC responses, e.g., when undergoing monoclonal antibody therapy.

Thus, provided herein are, among other things, methods for depleting B-cells in a patient, e.g., for treating a patient suffering from an autoimmune disorder, e.g., systemic lupus erythematosus (SLE).

Described herein are methods for treating a patient suffering from an autoimmune disorder, the method comprising administering a population of natural killer cells (NK cells) and an antibody targeted to an immune cell, wherein the NK cells are allogenic to the patient. In some embodiments, the immune cell is implicated in an autoimmune reaction. In some embodiments, the immune cell is a B cell. In some embodiments, the antibody is a B-cell depleting antibody, e.g., a B-cell depleting monoclonal antibody (mAb). In some embodiments, the antibody is an antibody targeted to human CD19 and/or human CD20. In some embodiments, the NK cells are KIR-B haplotype and homozygous for a CD16 158V polymorphism.

In some embodiments, the autoimmune disease is selected from, Acromegaly, Acquired aplastic anemia, Acquired hemophilia, Primary Agammaglobulinemia, Alopecia areata, Ankylosing spondylitis (AS). Anti-NMDA receptor encephalitis, Antiphospholipid syndrome (APS)|catastrophic antiphospholipid syndrome (CAPS)/Asherson's syndrome, Arteriosclerosis, Autoimmune Addison's disease (AAD), Autoimmune autonomic ganglionopathy (AAG)/autoimmune dysautonomia|autoimmune gastrointestinal dysmotility (AGID), Autoimmune encephalitis|acute disseminated encephalomvelitis (ADEM), Autoimmune gastritis, Autoimmune hemolytic anemia (AIHA), Autoimmune hepatitis (AIH), Autoimmune hyperlipidemia, Autoimmune hypophysitis, Autoimmune inner ear disease (AIED), Autoimmune lymphoproliferative syndrome (ALPS), Autoimmune myelofibrosis, Autoimmune myocarditis, Autoimmune oophoritis, Autoimmune pancreatitis (AIP), Autoimmune polyglandular syndromes, types I, II, & III (APS type 1. APS type 2, APS type 3, APECED), Autoimmune progesterone dermatitis, Autoimmune retinopathy (AIR), Autoimmune sudden sensorineural hearing loss (SNHL), Balo disease, Behget's disease, Birdshot chorioretinopathy/birdshot uveitis, Bullous pemphigoid, Castleman disease, Celiac disease, Chagas disease, Chronic inflammatory demyelinating polyneuropathy (CIDP), Chronic urticaria (CU), Churg-Strauss syndrome/eosinophilic granulomatosis with polyangiitis (EGPA), Cogan's syndrome, Cold agglutinin disease, CREST syndrome|limited cutaneous systemic sclerosis, Crohn's disease (CD), Cronkhite-Canada syndrome (CSS), Cryptogenic organizing pneumonia (COP), Dermatitis herpetiformis, Dermatomyositis, Type 1 Diabetes, Discoid lupus, Dressler's syndrome/postmyocardial infarction/postpericardiotomy syndrome, Eczema/Atopic Dermatitis, Endometriosis, Eosinophilic esophagitis, Eosinophilic fasciitis, Erythema nodosum, Essential mixed cryoglobulinemia, Evans syndrome. Fibrosing alveolitis/Idiopathic pulmonary fibrosis (IPF), Giant cell arteritis/temporal arteritis/Horton's disease, Giant Cell Myocarditis, Glomerulonephritis, Goodpasture's syndrome/anti-GBM/anti-TBM disease, Granulomatosis with polyangiitis (GPA)/Wegener's granulomatosis, Graves disease/thyroid eye disease. Guillain-Barré syndrome (GBS), Hashimoto's thyroiditis/chronic lymphocytic thyroiditis/autoimmune thyroiditis, Henoch-Schönlein purpura/IgA vasculitis, Hidradenitis suppurativa, Hurst's disease/acute hemorrhagic leukoencephalitis (AHLE), Hypogammaglobulinemia, IgA nephropathy/Berger's disease, Immune-mediated necrotizing myopathy (IMNM), Immune thrombocytopenia (ITP)/autoimmune thrombocytopenic purpura/autoimmune thrombocytopenia. Inclusion body myositis. IgG4-related sclerosing disease (ISD), Interstitial cystitis, Juvenile idiopathic arthritis/Adult-onset Still's disease, Juvenile polymyositis|Juvenile dermatomyositis|juvenile myositis, Kawasaki disease, Lambert-Eaton myasthenic syndrome (LEMS), Leukocytoclastic vasculitis, Lichen planus, Lichen sclerosus, Ligneous conjunctivitis. Linear IgA disease (LAD)|linear IgA bullous dermatosis (LABD), Lupus nephritis, Lyme disease/chronic Lyme disease/post-treatment Lyme disease syndrome (PTLDS), Lymphocytic colitis/microscopic colitis, Lymphocytic hypophystitis/autoimmune hypophystitis, Meniere's disease, Microscopic polyangiitis (MPA)/ANCA-associated vasculitis, Mixed connective tissue disease (MCTD), Mooren's ulcer, Mucha-Habermann disease, Multifocal motor neuropathy, Multiple sclerosis (MS), Myalgic encephalomyelitis (ME)/Chronic fatigue syndrome (CFS), Myasthenia gravis (MG), Narcolepsy, Neuromyelitis Optica/Devic's disease, Ocular cicatricial pemphigoid, Opsoclonus-mvoclonus syndrome (OMS). Palindromic rheumatism, Paraneoplastic cerebellar degeneration, Paraneoplastic pemphigus, Parry-Romberg syndrome (PRS)/Hemifacial atrophy (HFA)/Progressive facial hemiatrophy, Paroxysmal nocturnal hemoglobinuria (PNH), Peripheral uveitis/pars planitis, PANS/PANDAS, Parsonage-Turner syndrome, Pemphigus gestationis/herpes gestationis, Pemphigus foliaceus, Pemphigus vulgaris, Pemicious anemia. POEMS syndrome, Polyarteritis nodosa, Polymyalgia rheumatica, Polymyositis, Postural orthostatic tachycardia syndrome (POTS), Primary biliary cirrhosis (PBC)/primary biliary cholangitis, Primary sclerosing cholangitis (PSC), Psoriasis, Palmoplantar Pustulosis. Psoriatic arthritis, Pulmonary fibrosis, idiopathic (IPF), Pure red cell aplasia (PRCA), Pyoderma gangrenosum, Rasmussen's encephalitis. Raynaud's syndrome/phenomenon, Reactive arthritis/Reiter's syndrome, Reflex sympathetic dystrophy syndrome (RSD)/Complex regional pain syndrome (CRPS), Relapsing polychondritis, Restless leg syndrome (RLS)/Willis-Ekbom disease. Rheumatic fever, Rheumatoid arthritis, Sarcoidosis, Schmidt syndrome/autoimmune polyendocrine syndrome type II, Scleritis. Scleroderma, Sclerosing Mesenteritis/Mesenteric Panniculitis, Serpiginous choroidopathy, Sjögren's syndrome, Stiff person syndrome (SPS), Small fiber sensory neuropathy, Systemic lupus erythematosus (SLE), Subacute bacterial endocarditis (SBE), Subacute cutaneous lupus, Susac syndrome, Sydenham's chorea. Sympathetic ophthalmia, Takayasu's arteritis (vasculitis), Testicular autoimmunity (vasculitis, orchitis), Tolosa-Hunt syndrome, Transverse Myelitis (TM), Tubulointerstitial nephritis uveitis syndrome (TINU), Ulcerative colitis (UC), Undifferentiated connective tissue disease (UCTD), Uveitis|anterior/intermediate/posterior, Vasculitis. VEXAS Syndrome, Vitiligo, Vogt-Koyanagi-Harada syndrome (VKH), and combinations thereof.

In some embodiments, the autoimmune disorder is Systemic lupus erythematosus (SLE). In some embodiments, the patient has lupus nephritis. In some embodiments, the patient has relapsed after treatment with an anti-CD19 and/or anti-CD20 antibody. In some embodiments, the patient has experienced disease progression after treatment with autologous stem cell transplant or chimeric antigen receptor T-cell therapy (CAR-T).

In some embodiments, the patient is administered 1×10\hat{ }8 to 1×10\hat{ }10 NK cells. In some embodiments, the patient is administered 1×10\hat{ }9 to 8×10\hat{ }9 NK cells. The method of any one of the foregoing claims, wherein the patient is administered 4×10\hat{ }8, 1×10\hat{ }9, 4×10\hat{ }9, or 8×10\hat{ }9 NK cells. In some embodiments, the patient is administered 5×10\hat{ }8, 1×10\hat{ }9, or 4×10\hat{ }9 NK cells.

In some embodiments, the antibody is selected from Table 1, Table 2, or Table 3. In some embodiments, the antibody is rituximab, obinutuzumab, or tafasitamab. In some embodiments, the antibody is rituximab. In some embodiments, the antibody is Obinutuzumab. In some embodiments, the antibody is tafasitamab.

In some embodiments, the patient is administered a population of from 1×10\hat{ }9-5×10\hat{ }9 NK cells. In some embodiments, the patient is administered a population of 2×10\hat{ }9 NK cells, or thereabout, a population of 4×10\hat{ }9 NK cells, or thereabout, a population of 5×10\hat{ }8 NK cells, or thereabout, or a population of 1×10\hat{ }9 NK cells, or thereabout.

In some embodiments the patient is administered from 500 to 1500 mg of the antibody, or thereabout. In some embodiments, the patient is administered 100 mg of the antibody, or thereabout.

In some embodiments, the patient is subjected to lymphodepleting chemotherapy prior to treatment. In some embodiments, the lymphodepleting chemotherapy is non-myeloablative chemotherapy. In some embodiments, the lymphodepleting chemotherapy comprises treatment with at least one of cyclophosphamide and fludarabine. In some embodiments, the lymphodepleting chemotherapy comprises treatment with cyclophosphamide and fludarabine. In some embodiments, the cyclophosphamide is administered between 100 and 500 mg/m2/day. In some embodiments, the cyclophosphamide is administered at 250 or 300 mg/m2/day. In some embodiments, the cyclophosphamide is administered at 500 mg/m2/day. In some embodiments, the fludarabine is administered between 10 and 50 mg/m2/dav. In some embodiments, the fludarabine is administered 30 mg/m2/day.

In some embodiments, the method further comprises administering IL-2. In some embodiments, the patient is administered 1×10\hat{ }6 IU/m2 of IL-2. In some embodiments, the patient is administered 6 million IU of IL-2. In some embodiments, administration of IL-2 occurs within 1-4 hrs of administration of the NK cells. In some embodiments, the administration of the NK cells and the antibody occurs weekly. In some embodiments, the NK cells and the antibody are administered weekly for 3 to 8 or 4 to 8 weeks. In some embodiments, lymphodepletion occurs on days 1, 2, and 3 of a treatment cycle. In some embodiments, the NK cells are administered on days 6, 13, and 20 or 6, 9, 13 and 16 of a treatment cycle. In some embodiments, the NK cells are administered at or at about 2 billion or 4 billion cells per administration. In some embodiments, if administered, the NK cells are administered at or at about 4 billion cells on days 6, and 13. In some embodiments, if administered, the NK cells are administered at or at about 2 billion cells on days 9, 16, and 20. In some embodiments, the NK cells are administered at or at about 5×10\hat{ }8, 1×10\hat{ }9, or 4×10\hat{ }9 NK cells on days 6, 13, and 20. In some embodiments, the antibody is administered on days 2 and 13 of a treatment cycle. In some embodiments, the administration of the NK cells occurs weekly and the administration of the antibody occurs every other week.

In some embodiments, the NK cells are not genetically modified.

In some embodiments, at least 70% of the NK cells are CD56+ and CD16+. In some embodiments, at least 85% of the NK cells are CD56+ and CD3−. In some embodiments. 1% or less of the NK cells are CD3+. 1% or less of the NK cells are CD19+ and 1% or less of the NK cells are CD14+. In some embodiments, each administration of NK cells is administration of 1×10\hat{ }9 to 5×10\hat{ }9 NK cells. In some embodiments, each administration of NK cells is administration of 1×10\hat{ }9 to 5×10\hat{ }9 NK cells.

In some embodiments, the patient receives a dose of the CD20 targeted antibody before the first dose of NK cells.

In some embodiments, the expanded natural killer cells are expanded umbilical cord blood natural killer cells.

In some embodiments, the population of expanded natural killer cells comprises at least 60%, e.g., at least 70%, at least 80%, at least 90% at least 95%, at least 99%, or 100% CD16+ cells. In some embodiments, the population of expanded natural killer cells comprises at least 60%, e.g., at least 70%, at least 80%, at least 90% at least 95%, at least 99%, or 100% NKG2D+ cells. In some embodiments, the population of expanded natural killer cells comprises at least 60%. e.g., at least 70%, at least 80%, at least 90% at least 95%, at least 99%, or 100% NKp46+ cells. In some embodiments, the population of expanded natural killer cells comprises at least 60%, e.g., at least 70%, at least 80%, at least 90% at least 95%, at least 99%, or 100% NKp30+ cells. In some embodiments, the population of expanded natural killer cells comprises at least 60%, e.g., at least 70%, at least 80%, at least 90% at least 95%, at least 99%, or 100% DNAM-l+ cells. In some embodiments, the population of expanded natural killer cells comprises at least 60%, e.g., at least 70%, at least 80%, at least 90% at least 95%, at least 99%, or 100% NKp44+ cells. In some embodiments, the population of expanded natural killer cells comprises less than 20%. e.g., 10% or less, 5% or less, 1% or less, 0.5% or less, or 0% CD3+ cells. In some embodiments, the population of expanded natural killer cells comprises less than 20% or less, e.g., 10% or less, 5% or less, 1% or less, 0.5% or less, or 0% CD14+ cells. In some embodiments, the population of expanded natural killer cells comprises less than 20% or less, e.g., 10% or less. 5% or less. 1% or less, 0.5% or less, or 0% CD19+ cells. In some embodiments, the population of expanded natural killer cells comprises less than 20% or less, e.g., 10% or less, 5% or less, 1% or less, 0.5% or less, or 0% CD38+ cells.

In some embodiments, the natural killer cells do not comprise a CD16 transgene. In some embodiments, the natural killer cells do not express an exogenous CD16 protein. In some embodiments, the expanded natural killer cells are not genetically engineered. In some embodiments, the expanded natural killer cells are derived from the same umbilical cord blood donor.

In some embodiments, the population of NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion. 20 billion, 25 billion, 50 billion. 75 billion, 80 billion. 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

In some embodiments, the population of NK cells is produced by a method comprising: (a) obtaining seed cells comprising natural killer cells from umbilical cord blood; (b) depleting the seed cells of CD3+ cells; (c) expanding the natural killer cells by culturing the depleted seed cells with a first plurality of Hut78 cells engineered to express a membrane bound IL-21, a mutated TNFα, and a 4-1BBL gene to produce expanded natural killer cells, thereby producing the population of expanded natural killer cells.

In some embodiments, the population of NK cells is produced by a method comprising: (a) obtaining seed cells comprising natural killer cells from umbilical cord blood; (b) depleting the seed cells of CD3+ cells; (c) expanding the natural killer cells by culturing the depleted seed cells with a first plurality of Hut78 cells engineered to express a membrane bound IL-21, a mutated TNFα, and a 4-1BBL gene to produce a master cell bank population of expanded natural killer cells; and (d) expanding the master cell bank population of expanded natural killer cells by culturing with a second plurality of Hut78 cells engineered to express a membrane bound IL-21, a mutated TNFα, and a 4-1BBL gene to produce expanded natural killer cells; thereby producing the population of expanded natural killer cells.

In some embodiments, the population of NK cells is produced by a method further comprising, after step (c), (i) freezing the master cell bank population of expanded natural killer cells in a plurality of containers; and (ii) thawing a container comprising an aliquot of the master cell bank population of expanded natural killer cells, wherein expanding the master cell bank population of expanded natural killer cells in step (d) comprises expanding the aliquot of the master cell bank population of expanded natural killer cells.

In some embodiments, the umbilical cord blood is from a donor with the KIR-B haplotype and homozygous for the CD16 158V polymorphism.

In some embodiments, the population of NK cells is produced by a method comprising expanding the natural killer cells from umbilical cord blood at least 10.000 fold, e.g., 15,000 fold, 20,000 fold, 25.000 fold, 30,000 fold, 35,000 fold, 40,000 fold, 45,000 fold, 50,000 fold, 55,000 fold, 60,000 fold, 65,000 fold, or 70,000 fold.

In some embodiments, the population of expanded natural killer cells is not enriched or sorted after expansion.

In some embodiments, the percentage of NK cells expressing CD16 in the population of expanded natural killer cells is the same or higher than the percentage of natural killer cells in the seed cells from umbilical cord blood. In some embodiments, the percentage of NK cells expressing NKG2D in the population of expanded natural killer cells is the same or higher than the percentage of natural killer cells in the seed cells from umbilical cord blood. In some embodiments, the percentage of NK cells expressing NKp30 in the population of expanded natural killer cells is the same or higher than the percentage of natural killer cells in the seed cells from umbilical cord blood. In some embodiments, the percentage of NK cells expressing NKp44 in the population of expanded natural killer cells is the same or higher than the percentage of natural killer cells in the seed cells from umbilical cord blood. In some embodiments, the percentage of NK cells expressing NKp46 in the population of expanded natural killer cells is the same or higher than the percentage of natural killer cells in the seed cells from umbilical cord blood. In some embodiments, the percentage of NK cells expressing DNAM-1 in the population of expanded natural killer cells is the same or higher than the percentage of natural killer cells in the seed cells from umbilical cord blood.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative and are not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings of which:

FIG. 1 is a set of plots showing that AB-101 in combination with anti-CD20 antibody mediates killing of healthy donor B cells in vitro.

FIG. 2 is a graph showing that AB-101 in combination with anti-CD19 or anti-CD20 antibody mediates killing of healthy donor B cells in vitro.

FIG. 3 is a representative set of FACS plots of SLE B-cell apoptosis

FIG. 4 shows specific AB-101-mediated lysis of SLE B-cells in combination with anti-CD19 or anti-CD20 mAbs. Four sets of four bars for each antibody show % caspase positive B cells for each AB101:PBMC ratio (from left to right: 0, 0.2, 1, and 2), for each antibody level (from left to right: no antibody, 0.01 μg/mL, 0.1 μg/mL, and 1 μg/mL antibody).

FIG. 5 is a set of plots showing that AB-101 in combination with anti-CD20 antibody results in minimal killing of T cells.

FIG. 6 is a set of plots showing that AB-101 in combination with anti-CD20 antibody mediates killing of SLE donor B cells in a 4-hour cytotoxicity assay.

FIG. 7 shows enhanced SLE B cell killing observed with the combination of rituximab and AB-101. SLE patient PBMCs were isolated from peripheral blood and combined with thawed AB-101, with or without the anti-CD20 antibody, rituximab, for 4 hours. The percentage of caspase positive B cells was determined by flow cytometry. Data are expressed as the mean+/−SD of duplicate wells. Representative data is shown from one SLE patient sample. Four sets of four bars show % caspase positive B cells for each AB101:PBMC ratio (from left to right: 0, 0.2, 1, and 2), for each antibody level (from left to right: no antibody, 0.01 μg/mL, 0.1 μg/mL, and 1 μg/mL antibody).

FIG. 8 shows an example of a treatment regimen. DLT=dose-limiting toxicity; FU=follow up; EOT=end of treatment; Flu=fludarabine; Ritux=rituximab; Obi=obinutuzumab: CRR=complete renal response: Cyclo=cyclophosphamide.

FIG. 9 shows enhanced SLE B cell killing observed with the combination of Obinutuzumab and AB-101. SLE patient PBMCs were isolated from peripheral blood and combined with thawed AB-101, with or without the anti-CD20 antibody, obinutuzumab, for 4 hours. The percentage of caspase positive B-cells was determined by flow cytometry. Data are expressed as the mean+/−SD of duplicate wells. Representative data is shown from one SLE patient sample. Four sets of four bars for each antibody show % caspase positive B cells for each AB101:PBMC ratio (from left to right: 0, 0.2, 1, and 2), for each antibody level (from left to right: no antibody, 0.01 μg/mL, 0.1 μg/mL, and 1 μg/mL antibody).

FIG. 10 shows a dosing schematic for a humanized NSG model. Obin=Obinutuzumab.

FIG. 11 shows in vivo activity of AB-101 and Obinutuzumab in a humanized NSG model. HuNSG mice were administered the following treatments: vehicle (PBS IP, Freeze media IV), Obinutuzumab (150 μg/kg), AB-101 (1×10⁷ cells), or AB-101 plus obinutuzumab on study Day 0 or Days 0 and 7. Mice were bled at baseline (i.e., prior to the first treatment) and at indicated time points (A). Kinetics of peripheral blood B-cell counts by flow cytometry analysis are represented as the percent change of human CD19+B cells (cells/μl) from baseline±SEM (B). B-cells were gated on CD19 rather than CD20 due to the potential blockade of the CD20 detection reagent for flow cytometry. Data on Day 7 is following a single dose of Obin, AB-101 or obin+AB-101 and Day 14 is following two doses of obin, AB-101 or obin+AB-101 for the indicated groups. Obin=obinutuzumab. Six sets of six bars show % of baseline (cells/μL) for each days post-treatment (from left to right: 7, 14, 21, and 28), for each treatment (from left to right: vehicle, Obin (D0), Obin (D0, D7), AB-101 (D0, D7), Obin+AB-101 (D0). Obin+AB-101 (D0, D7).

FIG. 12 shows body weight change in a humanized NSG model. The mean (±SD) body weight change is shown for all groups, there is no significant difference in percent body weight change between vehicle, Obin, AB-101 or combination of Obin+AB-101 treatment groups. SD, standard deviation.

FIG. 13 shows the pharmacokinetic profile of AB-101. Distribution of AB-101 in several tissues of NSG mouse was determined by calculating amount of AB-101 DNA per μg of mouse blood/tissue DNA. Data is shown as mean concentration (+s.e.m.) of AB-101 DNA in each organ and is representative of 6 mice (3 male, 3 female) per each timepoint.

FIG. 14 shows SLE and healthy donor B cell characterization. B cell subsets from SLE and healthy donors were assessed by flow cytometry using gating shown in the top row. Data shows the average ±SEM. *p=<0.05.

FIG. 15 shows SLE and healthy donor NK cell characterization. NK cell subsets and surface markers from SLE and healthy donors were characterized by flow cytometry. Data shows the mean±SEM. *p<0.05, **p<0.005

### DETAILED DESCRIPTION

Provided herein are, amongst other things, Natural Killer (NK) cells, e.g., expanded and stimulated NK cells, methods for producing the NK cells, pharmaceutical compositions comprising the NK cells, and methods of treating patients suffering, e.g., from an autoimmune disorder, with the NK cells.

### I. EXPANSION AND STIMULATION OF NATURAL KILLER CELLS

In some cases, the NK cells are expanded and stimulated, e.g., as described in WO2022216813, which is hereby incorporated by reference in its entirety.

In some cases, e.g., after having been ex vivo expanded and stimulated, e.g., as described herein, the expanded and stimulated NK cell populations not only have a number/density (e.g., as described above) that could not occur naturally in the human body, but they also differ in their phenotypic characteristics, (e.g., gene expression and/or surface protein expression) with the starting source material or other naturally occurring populations of NK cells.

In some cases, the starting NK cell source is a sample derived from a single individual, e.g., a single cord blood unit that has not been ex vivo expanded. Therefore, in some cases, the expanded and stimulated NK cells share a common lineage, i.e., they all result from expansion of the starting NK cell source, and, therefore, share a genotype via clonal expansion of a population of cells that are, themselves, from a single organism. Yet, they could not occur naturally at the density achieved with ex vivo expansion and also differ in phenotypic characteristics from the starting NK cell source.

In some cases, the population of expanded and stimulated NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion. 500 billion, 600 billion, 700 billion, 800 billion, 900 billion, I trillion, 2 trillion, 3 trillion. 4 trillion. 5 trillion. 6 trillion, 7 trillion, 8 trillion, 9 trillion, or 10 trillion expanded natural killer cells.

In some embodiments, the expanded and stimulated NK cells comprise at least 80%, e.g., at least 90%, at least 95%, at least 99%, or 100% CD56+CD3− cells.

In some embodiments, the expanded and stimulated NK cells do not comprise a CD16 transgene.

In some embodiments, the expanded and stimulated NK cells do not express an exogenous CD16 protein.

The expanded and stimulated NK cells can be characterized, for example, by surface expression. e.g., of one or more of CD16. CD56, CD3, CD38, CD14. CD19. NKG2D.

NKp46, NKp30, DNAM-1, and NKp44.

The surface protein expression levels stated herein, in some cases are achieved without positive selection on the particular surface protein referenced. For example, in some cases, the NK cell source, e.g., a single cord unit, comprises both the KIR B allele of the KIR receptor family and the 158 V/V variant of CD16 and is + enriched and CD3(+) depleted, e.g., by gating on CD56+CD3− expression, but no other surface protein expression selection is carried out during expansion and stimulation.

In some embodiments, the expanded and stimulated NK cells. e.g., from expansion and stimulation of a single cord blood unit, e.g., as described above, comprise at least 60%, e.g., at least 70%, at least 80%, at least 90% at least 95%, at least 99%, or 100% NKG2D+ cells.

In some embodiments, the expanded and stimulated NK cells, e.g., from expansion and stimulation of a single cord blood unit. e.g., as described above, comprise at least 60%, e.g., at least 70%, at least 80%, at least 90% at least 95%, at least 99%, or 100% NKp46+ cells.

In some embodiments, the expanded and stimulated NK cells, e.g., from expansion and stimulation of a single cord blood unit. e.g., as described above, comprise at least 60%, e.g., at least 70%, at least 80%, at least 90% at least 95%, at least 99%, or 100% NKp30+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% DNAM-1+ cells.

In some embodiments, the expanded and stimulated NK cells. e.g., from expansion and stimulation of a single cord blood unit, e.g., as described above, comprise at least 60%, e.g., at least 70%, at least 80%, at least 90% at least 95%, at least 99%, or 100% NKp44+ cells.

In some embodiments, the expanded and stimulated NK cells. e.g., from expansion and stimulation of a single cord blood unit, e.g., as described above, comprise at least 60%, e.g., at least 70%, at least 80%, at least 90% at least 95%, at least 99%, or 100% CD94+(KLRD1) cells.

In some embodiments, the expanded and stimulated NK cells. e.g., from expansion and stimulation of a single cord blood unit. e.g., as described above, comprises less than or equal to 20%, e.g., less than or equal to 10%, less than or equal to 5%, less than or equal to 1% or 0% CD3+ cells.

In some embodiments, the expanded and stimulated NK cells, e.g., from expansion and stimulation of a single cord blood unit. e.g., as described above, comprises less than or equal to 20%, e.g., less than or equal to 10%, less than or equal to 5%, less than or equal to 1% or 0% CD14+ cells.

In some embodiments, the expanded and stimulated NK cells, e.g., from expansion and stimulation of a single cord blood unit. e.g., as described above, comprises less than or equal to 20%, e.g., less than or equal to 10%, less than or equal to 5%, less than or equal to 1% or 0% CD19+ cells.

In some embodiments, the expanded and stimulated NK cells, e.g., from expansion and stimulation of a single cord blood unit. e.g., as described above, comprises less than or equal to 20%, e.g., less than or equal to 10%, less than or equal to 5%, less than or equal to 1% or 0% CXCR+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD122+(IL2RB) cells.

In some embodiments, the expanded and stimulated NK cells, e.g., from expansion and stimulation of a single cord blood unit. e.g., as described above, comprises 90% or more, e.g., 91%, 92%, 93%, 94%, 95%, 96%, 97%, 98%, 99%, or 100% CD3−CD14−CD19−CD16+CD56− cells.

As described herein, the inventors have demonstrated that, surprisingly, the NK cells expanded and stimulated by the methods described herein express CD16 at high levels throughout the expansion and stimulation process, resulting in a cell population with high CD16 expression. The high expression of CD16 obviates the need for engineering the expanded cells to express CD16, which is important for initiating ADCC, and, therefore, a surprising and unexpected benefit of the expansion and stimulation methods described herein.

Thus, in some embodiments, the expanded and stimulated NK cells, e.g., from expansion and stimulation of a single cord blood unit, e.g., as described above, comprise 50% or more, e.g., 55%, 60%, 65%, 70%, 75%, 80%, 85%, 90%, or 95% CD16+NK cells.

In some embodiments, the expanded and stimulated NK cells, e.g., from expansion and stimulation of a single cord blood unit. e.g., as described above, comprises both the KIR B allele of the KIR receptor family and the 158 V/V variant of CD16 and comprise 50% or more, e.g., 55%, 60%, 65%, 70%, 75%, 80%, 85%, 90%, or 95% CD16+NK cells.

In some embodiments, the percentage of expanded and stimulated NK cells, e.g., from expansion and stimulation of a single cord blood unit, e.g., as described above, expressing CD16 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKG2D is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp30 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing DNAM-1 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp44 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp46 is the same or higher than the percentage of natural killer cells in the seed cells from umbilical cord blood.

As described herein, the inventors have also demonstrated that, surprisingly, the NK cells expanded and stimulated by the methods described herein express CD38 at low levels. CD38 is an effective target for certain cancer therapies (e.g., multiple myeloma and acute myeloid leukemia). See, e.g., Jiao et al., “CD38: Targeted Therapy in Multiple Myeloma and Therapeutic Potential for Solid Cancers,” Expert Opinion on Investigational Drugs 29(11):1295-1308 (2020).

Thus, in some embodiments, the expanded and stimulated NK cells. e.g., from expansion and stimulation of a single cord blood unit. e.g., as described above, comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit. e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells. e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells, and 50% or more, e.g., 55%, 60%, 65%, 70%, 75%, 80%, 85%, 90%, or 95% CD16+NK cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise: i) 50% or more, e.g., 55%, 60%, 65%, 70%, 75%, 80%, 85%, 90%, or 95% CD16+NK cells; and/or ii) less than or equal to 80% CD38+ cells, e.g., less than or equal to 75%, 70%, 65%, 60%, 55%, 50%, 45%, 40%, 35%, 30%, 25%, or 20% CD38+ cells; and/or iii) at least 60%, e.g., at least 70%, at least 80%, at least 90% at least 95%, at least 99%, or 100% NKG2D+ cells; and/or iv) at least 60%, e.g., at least 70%, at least 80%, at least 90% at least 95%, at least 99%, or 100% NKp46+ cells; and/or v) at least 60%, e.g., at least 70%, at least 80%, at least 90% at least 95%, at least 99%, or 100% NKp30+ cells; and/or vi) at least 60%, e.g., at least 70%, at least 80%, at least 90% at least 95%, at least 99%, or 100% DNAM-1+ cells; and/or vii) at least 60%, e.g., at least 70%, at least 80%, at least 90% at least 95%, at least 99%, or 100% NKp44+ cells; and/or viii) at least 60%, e.g., at least 70%, at least 80%, at least 90% at least 95%, at least 99%, or 100% CD94+(KLRD1) cells; and/or ix) less than or equal to 20%, e.g., less than or equal to 10%, less than or equal to 5%, less than or equal to 1% or 0% CD3+ cells; and/or x) less than or equal to 20%, e.g., less than or equal to 10%, less than or equal to 5%, less than or equal to 1% or 0% CD14+ cells; and/or xi) less than or equal to 20%, e.g., less than or equal to 10%, less than or equal to 5%, less than or equal to 1% or 0% CD19+ cells; and/or xii) less than or equal to 20%, e.g., less than or equal to 10%, less than or equal to 5%, less than or equal to 1% or 0% CXCR+ cells; and/or xiii) less than or equal to 20%. e.g., less than or equal to 10%, less than or equal to 5%, less than or equal to 1% or 0% CD122+(IL2RB) cells.

In some embodiments, the NK cell is engineered to alter, e.g., reduce, expression of one or more inhibitor receptor genes.

In some embodiments, the inhibitory receptor gene is a HLA-specific inhibitory receptor. In some embodiments, the inhibitory receptor gene is a non-HLA-specific inhibitory receptor.

In some embodiments, the inhibitor receptor gene is selected from the group consisting of KIR, CD94/NKG2A, LILRB1, PD-1, Irp60. Siglec-7. LAIR-1, and combinations thereof.

Also provided herein are pharmaceutical compositions comprising the natural killer cells described herein and dosage units of the pharmaceutical compositions described herein.

In some cases, the dosage unit comprises between 100 million and 1.5 billion cells, e.g., 100 million, 200 million, 300 million, 400 million, 500 million, 600 million, 700 million, 800 million, 900 million, 1 billion, 1.1 billion, 1.2 billion, 1.3 billion, 1.4 billion, or 1.5 billion.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

In some embodiments, the pharmaceutical composition comprises: a) natural killer cell(s) described herein; and b) a cryopreservation composition. Suitable cryopreservation compositions are described herein.

In some embodiments, the composition is frozen. In some embodiments, the composition has been frozen for at least three months, e.g., at least six months, at least nine months, at least 12 months, at least 15 months, at least 18 months, at least 24 months, or at least 36 months.

In some embodiments, at least 60%, e.g., at least 70%, at least 80%, at least 90% at least 95%, at least 99%, or 100% of the natural killer cells are viable after being thawed.

In some embodiments, the pharmaceutical composition comprises: a) a cryopreservation composition described herein; and b) therapeutic cell(s), e.g., the engineered NK cells described herein.

In some embodiments, the pharmaceutical composition further comprises: c) a buffer solution. Suitable buffer solutions are described herein, e.g., as for cryopreservation compositions.

In some embodiments, the pharmaceutical composition comprises from or from about 1×10⁷ to or to about 1×10⁹ cells/mL. In some embodiments, the pharmaceutical composition comprises 1×10⁸ cells/mL. In some embodiments, the pharmaceutical composition comprises about 1×10⁸ cells/mL.

In some embodiments, the pharmaceutical composition comprises from or from about 1×10⁸ to or to about 1×10¹⁰ cells/mL.

In some embodiments, the pharmaceutical composition further comprises an antibody or antigen binding fragment thereof, e.g., an antibody described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see. e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzvl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Examples of suitable pharmaceutical compositions are described, for example, in WO2017/135631 and WO2022/0133061, each of which is hereby incorporated by reference in its entirety.

### II. ANTIBODIES

The methods described herein comprise administering an antibody, e.g., an antibody that targets an immune cell, e.g., an immune cell implicated in an autoimmune reaction, e.g., a B cell. In some cases, the antibody is a B-cell depleting antibody, e.g., a B-cell depleting monoclonal antibody (mAb). In some cases, the antibody is a CD20 and/or CD19 targeted antibody. In some cases, the methods described herein comprise administering multiple antibodies, e.g., multiple antibodies that target immune cell(s), e.g., immune cell(s) implicated in an autoimmune reaction, e.g., a B cell. In some cases, one or more of the antibodies is a B-cell depleting antibody, e.g., a B-cell depleting monoclonal antibody (mAb). In some cases, the antibodies are selected from CD20 and/or CD19 targeted antibodies, e.g., as described herein.

The term “antibody” refers to an immunoglobulin molecule or immunologically active portion thereof, i.e., an antigen-binding portion.

The term “monoclonal antibody” as used herein refers to an antibody obtained from a population of substantially homogeneous antibodies, e.g., the individual antibodies comprising the population are identical except for possible naturally-occurring mutations that may be present in minor amounts. An antibody can be monoclonal. An antibody can be a human or humanized antibody. The term “monoclonal antibody” encompasses intact and full-length monoclonal antibodies as well as antibody fragments (e.g., Fab. Fab′, F(ab′)2, Fv), single chain antibodies (e.g., scFv), fusion proteins comprising an antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The term “chimeric antibody” refers to an antibody in which a portion of the heavy and/or light chain is derived from a first source or species, while the remainder of the heavy and/or light chain is derived from a different source or species.

The term “humanized antibody” as used herein refers to an antibody that comprises a human heavy chain variable region and a light chain variable region wherein the native CDR residues are replaced by residues from corresponding CDRs from a nonhuman antibody (e.g., mouse, rat, rabbit, or nonhuman primate), wherein the nonhuman antibody has the desired specificity, affinity, and/or activity. In some embodiments, one or more framework region residues of the human heavy chain or light chain variable regions are replaced by corresponding residues from nonhuman antibody. Furthermore, humanized antibodies can comprise residues that are not found in the human antibody or in the nonhuman antibody. In some embodiments, these modifications are made to further refine and/or optimize antibody characteristics. In some embodiments, the humanized antibody comprises at least a portion of an immunoglobulin constant region (e.g., CH1, CH2, CH3, Fc), typically that of a human immunoglobulin.

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, recombinant protein production, and B-cell hybridoma technology.

“Antibody fragments” can include a portion of an intact antibody, preferably the antigen binding or variable region of the intact antibody. Examples of antibody fragments include Fab, Fab′, F(ab′)2, and Fv, fragments; diabodies; linear antibodies; single-chain antibody molecules; and multispecific antibodies formed from antibody fragments.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of software bioinformatic tools available on the internet. X-ray crystallography may be used to characterize an epitope on a target protein by analyzing the amino acid residue interactions of an antigen/antibody complex.

“Fv” includes the minimum antibody fragment which contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. The Fab fragment also contains the constant domain of the light chain and the first constant domain (CH1) of the heavy chain. Fab fragments differ from Fab′ fragments by the addition of a few residues at the carboxy terminus of the heavy chain CHi domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgB1, IgG2, IgG3, IgG4, IgA, and IgA2. “Single-chain Fv” or “sFv” antibody fragments comprise the VH and VL domains of antibody, wherein these domains are present in a single polypeptide chain. In some cases, the Fv polypeptide further comprises a polypeptide linker between the VH and VL domains which enables the sFv to form the desired structure for antigen binding.

In various embodiments, the antibody or antigen binding fragment thereof comprises a human or humanized antibody. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. Methods for humanizing non-human antibodies are well known in the art.

An antibody that “binds to,” “specifically binds to,” or is “specific for” a particular polypeptide or an epitope on a particular polypeptide is one that binds to that particular polypeptide or epitope on a particular polypeptide without substantially binding to any other polypeptide or polypeptide epitope. The term “specifically binds” as used herein refers to a binding agent (e.g., antibody) that interacts more frequently, more rapidly, with greater duration, with greater affinity, or with some combination of the above to a particular antigen, epitope, protein, or target molecule than with alternative substances. A binding agent (e.g. antibody) that specifically binds an antigen can be identified, for example, by immunoassays, ELISAs, Surface Plasmon Resonance (SPR) assays (e.g., Biacore), or other techniques known to those of skill in the art. A binding agent that specifically binds an antigen binds the target antigen with a higher affinity than its affinity for a different antigen. The different antigen can be a related antigen. In some embodiments, a binding agent that specifically binds an antigen binds the target antigen with an affinity that is at least 20 times greater than its affinity for a different antigen, e.g., at least 30 times greater, at least 40 times greater, at least 50 times greater, at least 60 times greater, at least 70 times greater, at least 80 times greater, at least 90 times greater, or at least 100 times greater, than its affinity for a different antigen. In some embodiments, a binding agent that specifically binds a particular antigen binds a different antigen at such a low affinity that binding cannot be detected using an assay described herein or otherwise known in the art. In some embodiments, affinity is measured using SPR technology, e.g., in a Biacore system or other system known to those of skill in the art.

In some embodiments, the antibody or antigen-binding fragment thereof is an antibody. e.g., a full length antibody comprising an Fc domain including at least one heavy chain. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgA, IgD, IgE, IgG, or IgM antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, the antibody is an antibody fragment comprising an antigen-binding site. In some embodiments, the antibody is a scFv. In some embodiments, the antibody is a disulfide-linked scFv. In some embodiments, the antibody is a bispecific antibody or a multispecific antibody. In some embodiments, the antibody is a monovalent antibody. In some embodiments, the antibody is a monospecific antibody. In some embodiments, the antibody is a bivalent antibody. In some embodiments, the antibody is isolated. In some embodiments, the antibody is substantially pure. In some embodiments, a binding agent is a polyclonal antibody. Polyclonal antibodies can be prepared by any method known to those of skill in the art.

In some embodiments, a binding agent is a monoclonal antibody. Monoclonal antibodies can be prepared by any method known to those of skill in the art. In some embodiments, a binding agent is a humanized antibody. Various methods for generating humanized antibodies are known in the art. In some embodiments, a binding agent is a human antibody. Human antibodies can be prepared using various techniques known in the art.

In some embodiments, a binding agent is a scFv antibody, a Fv, a Fab, a F(ab′)2, a F(ab′), or a bispecific antibody.

In some embodiments, a bispecific antibody has decreased toxicity and/or side effects. In some embodiments, a bispecific antibody has decreased toxicity and/or side effects as compared to a mixture of the two individual antibodies or the antibodies as single agents. In some embodiments, a bispecific antibody has an increased therapeutic index. In some embodiments, a bispecific antibody has an increased therapeutic index as compared to a mixture of the two individual antibodies or the antibodies as single agents. Several techniques for making bispecific antibodies are known by those skilled in the art. In some embodiments, the bispecific antibodies comprise heavy chain constant regions with modifications in the amino acids that are part of the interface between the two heavy chains. These modifications are made to enhance heterodimer formation and generally reduce or eliminate homodimer formation. In some embodiments, the bispecific antibodies are generated using a knobs-into-holes (KIH) strategy. In some embodiments, the bispecific antibodies comprise variant hinge regions incapable of forming disulfide linkages between identical heavy chains (e.g., reduce homodimer formation). In some embodiments, the bispecific antibodies comprise heavy chains with changes in amino acids that result in altered electrostatic interactions. In some embodiments, the bispecific antibodies comprise heavy chains with changes in amino acids that result in altered hydrophobic/hydrophilic interactions. Bispecific antibodies can be intact antibodies or antibody fragments comprising antigen-binding sites.

Binding agents with more than two valencies are also contemplated. In some embodiments, trispecific or tetraspecific antibodies are generated.

In some cases, the antibody or antigen-binding fragment thereof is an IgG, IgA, or IgE antibody or antigen-binding fragment thereof. In some cases, the antibody or antigen-binding fragment thereof is an IgG antibody or antigen-binding fragment thereof. In some cases, the antibody or antigen-binding fragment thereof is an IgG1, IgG3, or IgG4 antibody or antigen-binding fragment thereof. In some cases, the antibody or antigen-binding fragment thereof is an IgG1 antibody or antigen-binding fragment thereof.

In some cases, the antibody or antigen binding fragment thereof is an antibody or combination of antibodies selected from Table 1, Table 2 or Table 3. In some cases, the antibody or antigen binding fragment thereof is an IgG antibody selected from Table 1, Table 2 or Table 3. In some cases, the antibody or antigen binding fragment thereof is an IgG1 antibody selected from Table 1, Table 2 or Table 3. In some cases, patient suffers from a disorder that the antibody or antigen-binding fragment thereof is approved as a therapeutic (e.g., by a regulatory agency such as the U.S. Food and Drug Administration or the European Medicines Agency). e.g., as reflected in Table 1, Table 2 or Table 3.

In some cases, the antibody or antigen-binding fragment thereof is engineered to enhance binding of its Fc domain to activating receptors (e.g., Fcγ-receptors), relative to antibody without the engineering.

TABLE 1
CD19 and CD20 Targeting Antibody Therapeutics Approved in US or EP. The Antibody
Society. Therapeutic monoclonal antibodies approved or in review in the EU or
US. (Apr. 17, 2023); antibodysociety.org/resources/approved-antibodies.

| International | | | Indication |
|---|---|---|---|
| non-proprietary | | | first |
| name, US product | Brand | Target; | approved |
| identifier | name | Format | or reviewed |
| Tositumomab- | Bexxar | CD20; Murine IgG2a | Non-Hodgkin lymphoma |
| I131 | | | |
| Rituximab | MabThera, | CD20; Chimeric IgG1 | Non-Hodgkin lymphoma |
| | Rituxan | | |
| Ibritumomab | Zevalin | CD20; Murine IgG1 | Non-Hodgkin lymphoma |
| tiuxetan | | | |
| Ofatumumab | Arzerra | CD20; Human IgG1 | Chronic lymphocytic |
| | | | leukemia |
| Obinutuzumab | Gazyva, | CD20; Humanized IgG1 | Chronic lymphocytic |
| | Gazyvaro | Glycoengineered | leukemia |
| Blinatumomab | Blincyto | CD19, CD3; Murine | Acute lymphoblastic |
| | | bispecific tandem scFv | leukemia |
| Ocrelizumab | OCREVUS | CD20; Humanized IgG1 | Multiple sclerosis |
| Tafasitamab, | Monjuvi, | CD19; Humanized IgG1 | Diffuse large B-cell |
| tafasitamab-cxix | Minjuvi | | lymphoma |
| Inebilizumab, | Uplizna | CD19; Humanized IgG1 | Neuromyelitis optica |
| inebilizumab- | | | spectrum disorders |
| cdon | | | |
| Loncastuximab | Zynlonta | CD19; Humanized IgG1 | Diffuse large B-cell |
| tesirine, | | ADC | lymphoma |
| loncastuximab | | | |
| tesirine-lpyl | | | |
| Ublituximab | BRIUMVI | CD20; Chimeric IgG1 | Multiple sclerosis |
| Tebentafusp, | KIMMTRAK ® | gp100, CD3; Bispecific | Metastatic uveal |
| tebentafusp-tebn | | immunoconjugate | melanoma |
| Relatlimab | Opdualag | LAG-3; Human IgG4 | Melanoma |
| | (relatlimab + | | |
| | nivolumab | | |
| | combo) | | |
| Mosunetuzumab | Lunsumio | CD20, CD3; | Follicular lymphoma |
| | | Humanized IgG1 | |
| | | bispecific | |
| Epcoritamab | (Pending) | CD20, CD3; | Large B-cell lymphoma |
| | | Humanized IgG1k/l | |
| | | bispecific | |
| Glofitamab | (Pending) | CD20, CD3e; Bispecific | Diffuse large B-cell |
| | | 2 + 1 IgG1 CrossMab | lymphoma |

TABLE 2
Commercially sponsored monoclonal antibody therapeutics first approved
or undergoing regulatory review outside the European Union or
United States in 2022. Kaplon et al. (2023) Antibodies to watch
in 2023, mAbs, 15:1, DOI: 10.1080/19420862.2022.2153410

| INN | Target(s); | Indication first approved |
|---|---|---|
| (Brand name) | Format | or in review |
| Ripertamab | CD20; Chimeric | Diffuse large B- |
| (Anpingxi ®) | IgG1κ | cell lymphoma |

TABLE 3
CD20 Targeted Antibodies

| Name | Internal Name | Antigen | Company | Reference |
|---|---|---|---|---|
| ofatumumab | Arzerra, Kesimpta, | CD20 | Genmab, GSK, | Sorensen et al., Neurology. |
| | GSK1841157, | | Novartis | 2014 Feb. 18; 82(7): 573-81 |
| | HuMax-CD20, 2F2, | | | |
| | OMB157 | | | |
| ibritumomab | Zevalin, 2B8, C2B8, | CD20 | Biogen, CTI | Witzig et al., J Clin Oncol. |
| tiuxetan | Y2B8 | | Biopharma, | 2002 May 15; 20(10): 2453-63 |
| | | | Spectrum | |
| rituximab | Mab Thera, Rituxan, | CD20 | Biogen, Roche | Salles et al., Adv Ther. 2017 |
| | C2B8, IDEC-C2B8, | | | October; 34(10): 2232-2273 |
| | IDEC-102, RG105 | | | |
| obinutuzumab | Gazyvaro, Gazyva, | CD20 | Biogen, Genentech, | Marcus et al., N Engl J Med. |
| | GA101, GA-101, | | Glycart, Roche | 2017 Oct. 5; 377(14): 1331- |
| | RO5072759. | | | 1344 |
| | RG7159, R7159, | | | |
| | humanized B-Ly1, | | | |
| | afatuzumab | | | |
| tositumomab | Bexxar, I131 | CD20 | GSK | Zelenctz, Semin Oncol. |
| | tositumomab | | | 2003 April: 30(2 Suppl 4): |
| | | | | 22-30 |
| ocrelizumab | Ocrevus, 2H7.v16, | CD20 | Biogen, Genentech, | Kappos et al., Lancet. 2011 |
| | rhuMAb2H7, | | Xoma | Nov. 19: 378(9805): 1779-87 |
| | PRO70769, RG1594 | | | |
| GP2013 | Riximyo, SDZ- | CD20 | Novartis, Sandoz | Smolen et al., Ann Rheum |
| | RTX, Rixathon | | | Dis. 2017 September; 76(9): |
| | | | | 1598-1602 |
| rituximab-abbs | Truxima, Tuxella, | CD20 | Celltrion. Mundi | Coiffier, Expert Rev Clin |
| | Ritemvia, Blitzima, | | pharma | Pharmacol. 2017 |
| | CT-P10 | | | September; 10(9): 923-933 |
| BCD-020 | AcellBia, BCD020 | CD20 | Biocad | Poddubnaya et al., Hematol |
| | | | | Oncol. 2020 February; 38(1): |
| | | | | 67-73 |
| HLX01 | Hanlikon | CD20 | Shanghai Henlius | Shi et al., J Hematol Oncol. |
| | | | | 2020 Apr. 16; 13(1): 38 |
| IGN002 | | CD20 | ImmunGene, Val | Trial ID: NCT02847949 |
| | | | or Bio | |
| MK-8808 | | CD20 | Merck (MSD) | Trial ID: NCT01370694 |
| plamotamab | XmAb13676, | CD20, | Novartis, Xencor | Trial ID: NCT02924402 |
| | XENP13676 | CD3 | | |
| MT-3724 | | CD20 | Molecular | Huang et al., Blood Cancer |
| | | | Templates | J. 2018 Mar. 20; 8(3): 33 |
| RGB-03 | | CD20 | Gedeon Richter | Trial ID: NCT02371096 |
| IGM-2323 | | CD20, | IGM Bio | Trial ID: NCT04082936 |
| | | CD3 | | |
| CHO-H01 | | CD20 | Cho Pharma | Trial ID: NCT03221348 |
| B001 | | CD20 | Shanghai Pharma | Trial ID: NCT03332121 |
| | | | Holdings | |
| BCD-132 | | CD20 | Biocad | Trial ID: NCT04056897 |
| Sunshine | | CD20 | Sunshine Guojian | Trial ID: NCT03980379 |
| Guojian 304 | | | Pharma | |
| IMM0306 | | CD20, | ImmuneOnco | Trial ID: NCT04746131 |
| | | CD47 | | |
| ocaratuzumab | AME 33, AME- | CD20 | AME, Lilly, | Cheney et al., MAbs. May- |
| | 133v, LY2469298 | | Mentrik | June 2014; 6(3): 749-55 |
| PRO131921 | | CD20 | Genentech | Casulo et al., Clin Immunol. |
| | | | | 2014 September; 154(1): 37-46 |
| TL011 | | CD20 | Teva | Trial ID: NCT01205737 |
| mosunetuzumab | BTCT4465A, | CD20, | Genentech | Hosseini et al., NPJ Syst |
| | RG7828, | CD3e | | Biol Appl. 2020 Aug. |
| | RO7030816 | | | 28; 6(1): 28 |
| 2B8T2M | ALT-803 | CD20, | Altor | Trial ID: NCT01946789 |
| | | IL-15 | | |
| MIL62 | | CD20 | Beijing | Trial ID: NCT04103905 |
| | | | Mabworks | |
| TQB2303 | | CD20 | Chia Tai | Trial ID: NCT03777085 |
| | | | Tianging Pharma | |
| SBI-087 | PF-05230895, | CD20 | Pfizer, Trubion | Damjanov et al., J |
| | 2LM20-4 | | | Rheumatol. 2016 |
| | | | | December; 43(12): 2094-2100 |
| TRU-015 | PF-5212374 | CD20 | Pfizer, Trubion | Burge et al., Clin Ther. 2008 |
| | | | | October; 30(10): 1806-16 |
| veltuzumab | hA20, IMMU-106 | CD20 | Immunomedics, | Goldenberg et al., Leuk |
| | | | Nycomed, Takeda | Lymphoma. 2010 |
| | | | | May; 51(5): 747-55 |
| odronextamab | REGN1979 | CD20, | Regeneron | Trial ID: NCT03888105 |
| | | CD3 | | |
| RO7082859 | CD20-TCB | CD20 | Roche | Trial ID: NCT03075696 |
| zuberitamab | HS006, | CD20 | Zheijang Hisun | Trial ID: NCT03485118 |
| | RHCACD20MA | | | |
| PBO-326 | | CD20 | Probiomed | Trial ID: NCT01277172 |
| ublituximab | LFB-R603, TGTX- | CD20 | LFB, TG | Fox et al., Mult Scler. 2021 |
| | 1101, TG-1101 | | Therapeutics | March; 27(3): 420-429 |
| Reditux | DRL RI | CD20 | Dr. Reddy's | Bhati et al., Clin Rheumatol. |
| | | | | 2016 August; 35(8): 1931-1935 |
| CMAB304 | Retuxira | CD20 | Shanghai CP | Trial ID: NCT01459887 |
| | | | Guojian | |
| PF-05280586 | Rituximab-Pfizer | CD20 | Pfizer | Sharman et al., BioDrugs. |
| | | | | 2020 April; 34(2): 171-181 |
| BI 695500 | | CD20 | Boehringer | Trial ID: NCT01950273 |
| ripertamab | SCT400 | CD20 | Sinocelltech | Trial ID: NCT02206308 |
| ABP 798 | ABP798 | CD20 | Amgen | Niederwieser et al., Target |
| | | | | Oncol. 2020 October; 15(5): |
| | | | | 599-611 |
| IBI301 | IBI301-A | CD20 | Innovent, Lilly | Jiang et al., Sci Rep. 2020 |
| | | | | Jul. 15; 10(1): 11676 |
| MabionCD20 | | CD20 | Mabion | Trial ID: NCT02617485 |
| RTXM83 | | CD20 | mAbxience | Cerutti et al., BioDrugs. |
| | | | | 2019 June: 33(3): 307-319 |
| SAIT101 | | CD20 | Samsung Bioepis | Trial ID: NCT02809053 |
| epcoritamab | GEN3013, | CD20, | Abbvie, Genmab | Van der Horst et al., Blood |
| | DuoBody- | CD3 | | Cancer J. 2021 Feb. |
| | CD3 × CD20 | | | 18; 11(2): 38 |
| GB241 | | CD20 | Genor | Trial ID: NCT03003039 |
| JHL1101 | | CD20 | JHL Biotech | Trial ID: NCT03670901 |

In some cases, the antibody or antigen-binding fragment thereof is an NK cell engager, e.g., a bispecific or trispecific antibody, that bridges NK cell activating receptor(s) (for example, CD16A, NKG2D, NKp30, or NKp46) and molecule(s) specific to disease cell (e.g., tumor cell(s)). See, e.g., Demaria et al., “Natural Killer Cell Engagers in Cancer Immunotherapy: Next Generation of Immuno-Oncology Treatments.” European Journal of Immunology 51(8):doi.org/10.1002/eji.202048953 (2021).

### III. PHARMACEUTICAL COMPOSITIONS

Provided herein are pharmaceutical compositions comprising the natural killer cells described herein and dosage units of the pharmaceutical compositions described herein.

In some cases, the dosage unit comprises between 100 million and 1.5 billion cells, e.g., 100 million, 200 million, 300 million, 400 million, 500 million, 600 million, 700 million, 800 million, 900 million, 1 billion, 1.1 billion, 1.2 billion, 1.3 billion, 1.4 billion, or 1.5 billion cells, or thereabout.

In some cases, the dosage unit comprises between 100 million and 10 billion cells, e.g., 100 million, 200 million, 300 million, 400 million, 500 million, 600 million, 700 million, 800 million, 900 million, 1 billion, 1.5 billion, 2 billion, 2.5 billion, 3 billion, 3.5 billion, 4 billion, 4.5 billion, 5 billion, 5.5 billion, 6 billion, 6.5 billion, 7 billion, 7.5 billion, 8 billion. 8.5 billion, 9 billion, 9.5 billion, 10 billion cells, or thereabout.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

In some embodiments, the pharmaceutical composition comprises: a) natural killer cell(s) described herein; and b) a cryopreservation composition.

Suitable cryopreservation compositions are described herein.

In some embodiments, the composition is frozen. In some embodiments, the composition has been frozen for at least three months, e.g., at least six months, at least nine months, at least 12 months, at least 15 months, at least 18 months, at least 24 months, or at least 36 months.

In some embodiments, at least 60%, e.g., at least 70%, at least 80%, at least 90% at least 95%, at least 99%, or 100% of the natural killer cells are viable after being thawed.

In some embodiments, the pharmaceutical composition comprises: a) a cryopreservation composition described herein; and b) therapeutic cell(s).

In some embodiments, the therapeutic cell(s) are animal cell(s). In some embodiments, the therapeutic cell(s) are human cell(s).

In some embodiments, the therapeutic cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof.

In some embodiments, the immune cell(s) are natural killer (NK) cells. In some embodiments, the natural killer cell(s) are expanded and stimulated by a method described herein.

In some embodiments, the pharmaceutical composition further comprises: c) a buffer solution. Suitable buffer solutions are described herein, e.g., as for cryopreservation compositions.

In some embodiments, the pharmaceutical composition comprises from or from about 1×10⁷ to or to about 1×10⁹ cells/mL. In some embodiments, the pharmaceutical composition comprises 1×10⁸ cells/mL. In some embodiments, the pharmaceutical composition comprises about 1×10⁸ cells/mL.

In some embodiments, the pharmaceutical composition comprises from or from about 1×10⁸ to or to about 1×10¹⁰ cells/mL.

In some embodiments, the pharmaceutical composition further comprises an antibody or antigen binding fragment thereof, e.g., an antibody described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see. e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents: antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polvetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

### IV. METHODS OF TREATMENT

The NK cells described herein find use for treating autoimmune disorders.

Thus, also provided herein are methods of treating a patient suffering from a disorder, e.g., a disorder associated with autoimmunity, e.g., autoreactive immune cells, e.g., autoreactive B cells, comprising administering the NK cells, e.g., the NK cells described herein, and a CD19 and/or CD20 targeting antibody, e.g., an antibody described herein.

As used herein, the terms “treatment,” “treat,” and “treating” refer to reversing, alleviating, delaying the onset of, or inhibiting the progress of a disorder associated with autoimmunity (e.g., a disorder described herein, e.g., systemic lupus erythematosus (“SLE”)). In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein. “delaying” development of a disease or disorder, or one or more symptoms thereof, means to defer, hinder, slow, retard, stabilize and/or postpone development of the disease, disorder, or symptom thereof. This delay can be of varying lengths of time, depending on the history of the disease and/or subject being treated. As is evident to one skilled in the art, a sufficient or significant delay can, in effect, encompass prevention, in that the subject does not develop the disease, disorder, or symptom thereof. For example, a method that “delays” development of an autoimmune disorder is a method that reduces the probability of disease development in a given time frame and/or reduces extent of the disease in a given time frame, when compared to not using the method. Such comparisons may be based on clinical studies, using a statistically significant number of subjects.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject and/or before a certain stage of the disease. The subject may be an individual at risk of developing the disease or disorder, or at risk of disease progression, e.g., development of proliferative lupus nephritis. Such as an individual who has one or more risk factors known to be associated with development or onset of the disease or disorder. For example, an individual may have mutations associated with the development or progression of an autoimmune disease (e.g., SLE). Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

In some cases, an “increased” or “enhanced” amount refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

In some cases, a “decreased” or “reduced” or “lesser” amount refers to a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

Also provided herein are methods of depleting immune cell(s), e.g., autoreactive immune cells, e.g., autoreactive B cells, comprising administering the NK cells, e.g., the NK cells described herein, and a CD19 and/or CD20 targeting antibody, e.g., an antibody described herein. In some cases, the immune cell is a CD19+ immune cell. e.g., a CD19+ autoreactive immune cell, e.g., a CD19+ autoreactive B cell. In some cases, the immune cell is a CD20+ immune cell, e.g., a CD20+ autoreactive immune cell, e.g., a CD20+ autoreactive B cell. In some cases, the immune cell is a CD19+/CD20−, CD19+/CD20+, or CD19−/CD20+ immune cell. e.g., a CD19+/CD20−, CD19+/CD20+, or CD19−/CD20+ autoreactive immune cell, e.g., a CD19+/CD20−, CD19+/CD20+, or CD19−/CD20+ autoreactive B cell.

In some cases, the immune cell(s) (e.g., the autoreactive immune cell(s)) comprise or consist of B cell(s) (e.g., autoreactive B cells). In some cases, the B cell(s) are CD19+/CD20−B cell(s), CD19+/CD20+B cell(s), CD19−/CD20+B cell(s), or a combination thereof.

In some cases, the B cell(s) are selected from B cell(s) of anorectum, B cell(s) of appendix, B cell(s) of medullary sinus of lymph node, lymph node mantle zone B cell(s), monocytoid B cell(s), CD19-positive B cell(s), and combinations thereof,

In some cases, the CD19-positive B cell(s) are selected from immature B cell(s), mature B cell(s), precursor B cell(s), transitional stage B cell(s), and combinations thereof.

In some cases, the immature B cell(s) are selected from CD38-negative immature B cell(s), fraction E immature B cell(s), and combinations thereof.

In some cases, the mature B cell(s) are selected from B-1 B cell(s), B-2 B cell(s). Be cell(s), Peyer's patch B cell(s), follicular B cell(s), fraction F mature B cell(s), germinal center B cell(s), marginal zone B cell(s) of lymph node, marginal zone B cell(s) of spleen, memory B cell(s), naïve B cell(s), plasmablast(s), regulatory B cell(s), and combinations thereof.

In some cases, the B-1 B cell(s) are selected from B-1a B cell(s), B-1b B cell(s), and combinations thereof. In some cases, the B-2 B cell(s) are selected from Peyer's patch B cell(s), follicular B cell(s), fraction F mature B cell(s), and combinations thereof. In some cases, the follicular B cell(s) are selected from Bm1 B cell(s). Bm2 B cell(s), and combinations thereof. In some cases, the fraction F mature B cell(s) are Bm1 B cell(s). In some cases, the Be cell(s) are selected from Bel cell(s), Be2 cell(s), and combinations thereof. In some cases, the germinal center B cell(s) are selected from Bm2′ B cell(s), Bm3 B cell(s), Bm3-delta B cell(s), Bm4 B cell(s), centroblast(s), centrocyte(s), tonsil germinal center B cell(s), and combinations thereof.

In some cases, the memory B cell(s) are selected from Bm5 B cell(s). IgD-negative memory B cell(s), IgM memory B cell(s), class switched memory B cell(s), double negative memory B cell(s), unswitched memory B cell(s), and combinations thereof.

In some cases, the IgD-negative memory B cell(s) are selected from Bm5 B cell(s), CD38-negative IgG memory B cell(s). IgD-negative CD38-positive IgG memory B cell(s), IgM memory B cell(s), double negative memory B cell(s), and combinations thereof In some cases, the double negative memory B cell(s) are selected from IgG-negative double negative memory B cell(s), IgG-positive double negative memory B cell(s), and combinations thereof.

In some cases, the class switched memory B cell(s) are selected from IgA memory B cell(s), IgE memory B cell(s), IgG memory B cell(s), IgG-negative class switched memory B cell9s), and combinations thereof. In some cases, the IgG memory B cell(s) are selected from CD38-negative IgG memory B cell(s), CD38-postiive IgG memory B cell(s), and combinations thereof. In some cases, the CD38-positive IgG memory B cell(s) are selected from IgD-negative CD38-positive IgG memory B cell(s), IgD-positive CD38-positive IgG memory B cell(s), and combinations thereof. In some cases, the IgG-negative class switched memory B cell(s) ae selected from CD38-positive IgG-negative class switched memory B cell(s), CD38-positive IgG-negative class switched memory B cell(s), and combinations thereof. In some cases, the CD38-negative IgG-negative class switched memory B cell(s) are selected from CD24-negative CD38-negative IgG-negative class switched memory B cell(s), CD24-positive CD38-negative IgG-negative class switched memory B cell(s), and combinations thereof. In some cases, the CD38-positive IgG-negative class switched memory B cell(s) are selected from B220-low CD38-positive IgG-negative class switched memory B cell(s), B220-positive CD38-positive IgG-negative class switched memory B cell(s), and combinations thereof. In some cases, the B220-positive CD38-positive IgG-negative class switched memory B cell(s) are B220-low CD38-positive IgG-negative class switched memory B cell(s). In some cases, the double negative memory B cell(s) are selected from IgG-negative double negative memory B cell(s), IgG-positive double negative memory B cell(s), and combinations thereof. In some cases, the unswitched memory B cell(s) are selected from CD38-negative unswitched memory B cell(s), CD38-positive unswitched memory B cell(s), and combinations thereof. In some cases, the CD38-negative unswitched memory B cell(s) are selected from B220-low CD38-negative unswitched memory B cell(s), B220-positive CD38-negative unswitched memory B cell(s), and combinations thereof. In some cases, the B220-positive CD38-negative unswitched memory B cell(s) are B220-low CD38-negative unswitched memory B cell(s). In some cases, the CD38-positive unswitched memory B cell(s) are selected from B220-low CD38-positive unswitched memory B cell(s), B220-positive CD38-positive unswitched memory B cell(s), and combinations thereof. In some cases, the B220-positive CD38-positive unstitched memory B cell(s) are B220-low CD38-positive unswitched memory B cell(s). In some cases, the naïve B cell(s) are selected from CD38-negative naïve B cell(s), CD38-positive naïve B cell(s), and combinations thereof. In some cases, the CD38-positive naïve B cell(s) are B220-low CD38-positive naïve B cell(s), B220-positive cd38-positive naïve B cell(s), and combinations thereof. In some cases, the B220-positive CD38-positive naïve B cell(s) are B220-low CD38-positive naïve B cell(s). In some cases, the plasmablast(s) are selected from CD86-positive plasmablast(s), IgA plasmablast(s), IgD plasmablast(s), IgD plasmablast(s), IgE plasmablast(s), IgG plasmablast(s), IgM plasmablast(s), and combinations thereof.

In some cases, the precursor B cell(s) are selected from fraction B/C precursor B cell(s), fraction C′ precursor B cell(s), fraction D precursor B cell(s), late pro-B cell(s), pre-B-I cell(s), pre-B-II cell(s), and combinations thereof. In some cases, the pre-B-II cell(s) are selected from large pre-B-II cell(s), small pre-B-II cell(s), and combinations thereof. In some cases, the large pre-B-II cell(s) are selected from preBCR-negative large pre-B-II cell(s), preBCR-positive large pre-B-II cell(s), and combinations thereof. In some cases, the pre-BCR-positive large pre-B-II cell(s) are CD38-high pre-BCR positive cell(s). In some cases, the small pre-B-II cell(s) are CD22-positive, CD38-low small pre-B cell(s).

In some cases, the transitional stage B cell(s) are selected from TI B cell(s), T2 B cell(s). T3 B cell(s), and combinations thereof.

### A. Disorders

Methods and manufactured compositions disclosed herein find use in targeting a number of disorders, such as autoimmune disorders. A benefit of the approaches herein is that allogenic cells are used in combination with exogenous antibody administration to specifically target immune cells. e.g., B cells.

In some cases, the autoimmune disorder is driven by autoantibodies and/or autoreactive B cells.

In some cases, the autoimmune disorder is selected from Acromegaly, Acquired aplastic anemia, Acquired hemophilia, Primary Agammaglobulinemia. Alopecia areata, Ankylosing spondylitis (AS), Anti-NMDA receptor encephalitis, Antiphospholipid syndrome (APS)|catastrophic antiphospholipid syndrome (CAPS)/Asherson's syndrome, Arteriosclerosis, Autoimmune Addison's disease (AAD), Autoimmune autonomic ganglionopathy (AAG)/autoimmune dysautonomia|autoimmune gastrointestinal dysmotility (AGID), Autoimmune encephalitis|acute disseminated encephalomyelitis (ADEM), Autoimmune gastritis, Autoimmune hemolytic anemia (AIHA), Autoimmune hepatitis (AIH), Autoimmune hyperlipidemia, Autoimmune hypophysitis, Autoimmune inner ear disease (AIED), Autoimmune lymphoproliferative syndrome (ALPS), Autoimmune myelofibrosis, Autoimmune myocarditis, Autoimmune oophoritis, Autoimmune pancreatitis (AIP), Autoimmune polyglandular syndromes, types I, IL. & III (APS type 1, APS type 2, APS type 3, APECED), Autoimmune progesterone dermatitis. Autoimmune retinopathy (AIR), Autoimmune sudden sensorineural hearing loss (SNHL), Balo disease, Behget's disease, Birdshot chorioretinopathy/birdshot uveitis, Bullous pemphigoid, Castleman disease, Celiac disease, Chagas disease, Chronic inflammatory demyelinating polyneuropathy (CIDP), Chronic urticaria (CU), Churg-Strauss syndrome/eosinophilic granulomatosis with polyangiitis (EGPA), Cogan's syndrome, Cold agglutinin disease, CREST syndrome|limited cutaneous systemic sclerosis, Crohn's disease (CD), Cronkhite-Canada syndrome (CSS), Cryptogenic organizing pneumonia (COP), Dermatitis herpetiformis, Dermatomyositis, Type 1 Diabetes, Discoid lupus, Dressler's syndrome/postmyocardial infarction/postpericardiotomy syndrome, Eczema/Atopic Dermatitis, Endometriosis, Eosinophilic esophagitis, Eosinophilic fasciitis, Erythema nodosum, Essential mixed cryoglobulinemia, Evans syndrome, Fibrosing alveolitis/Idiopathic pulmonary fibrosis (IPF), Giant cell arteritis/temporal arteritis/Horton's disease, Giant Cell Myocarditis, Glomerulonephritis, Goodpasture's syndrome/anti-GBM/anti-TBM disease. Granulomatosis with polyangiitis (GPA)/Wegener's granulomatosis, Graves disease/thyroid eye disease, Guillain-Barré syndrome (GBS), Hashimoto's thyroiditis/chronic lymphocytic thyroiditis/autoimmune thyroiditis, Henoch-Schönlein purpura/IgA vasculitis, Hidradenitis suppurativa, Hurst's disease/acute hemorrhagic leukoencephalitis (AHLE), Hypogammaglobulinemia, IgA nephropathy/Berger's disease, Immune-mediated necrotizing myopathy (IMNM), Immune thrombocytopenia (ITP)/autoimmune thrombocytopenic purpura/autoimmune thrombocytopenia, Inclusion body myositis, IgG4-related sclerosing disease (ISD), Interstitial cystitis, Juvenile idiopathic arthritis/Adult-onset Still's disease, Juvenile polymyositis|Juvenile dermatomyositis|juvenile myositis, Kawasaki disease, Lambert-Eaton myasthenic syndrome (LEMS), Leukocytoclastic vasculitis, Lichen planus, Lichen sclerosus, Ligneous conjunctivitis, Linear IgA disease (LAD)|linear IgA bullous dermatosis (LABD), Lupus nephritis, Lyme disease/chronic Lyme disease/post-treatment Lyme disease syndrome (PTLDS), Lymphocytic colitis/microscopic colitis, Lymphocytic hypophystitis/autoimmune hypophystitis, Meniere's disease, Microscopic polyangiitis (MPA)/ANCA-associated vasculitis, Mixed connective tissue disease (MCTD), Mooren's ulcer, Mucha-Habermann disease, Multifocal motor neuropathy, Multiple sclerosis (MS), Myalgic encephalomyelitis (ME)/Chronic fatigue syndrome (CFS), Myasthenia gravis (MG), Narcolepsy, Neuromyelitis Optica/Devic's disease, Ocular cicatricial pemphigoid, Opsoclonus-myoclonus syndrome (OMS). Palindromic rheumatism, Paraneoplastic cerebellar degeneration, Paraneoplastic pemphigus, Parry-Romberg syndrome (PRS)/Hemifacial atrophy (HFA)/Progressive facial hemiatrophy, Paroxysmal nocturnal hemoglobinuria (PNH), Peripheral uveitis/pars planitis, PANS/PANDAS, Parsonage-Turner syndrome, Pemphigus gestationis/herpes gestationis, Pemphigus foliaceus, Pemphigus vulgaris, Pemicious anemia. POEMS syndrome, Polyarteritis nodosa, Polymyalgia rheumatica, Polymyositis, Postural orthostatic tachycardia syndrome (POTS). Primary biliary cirrhosis (PBC)/primary biliary cholangitis, Primary sclerosing cholangitis (PSC), Psoriasis, Palmoplantar Pustulosis, Psoriatic arthritis, Pulmonary fibrosis, idiopathic (IPF), Pure red cell aplasia (PRCA), Pyoderma gangrenosum, Rasmussen's encephalitis, Raynaud's syndrome/phenomenon, Reactive arthritis/Reiter's syndrome, Reflex sympathetic dystrophy syndrome (RSD)/Complex regional pain syndrome (CRPS), Relapsing polychondritis, Restless leg syndrome (RLS)/Willis-Ekbom disease, Rheumatic fever, Rheumatoid arthritis, Sarcoidosis, Schmidt syndrome/autoimmune polyendocrine syndrome type II, Scleritis, Scleroderma, Sclerosing Mesenteritis/Mesenteric Panniculitis, Serpiginous choroidopathy, Sjögren's syndrome. Stiff person syndrome (SPS), Small fiber sensory neuropathy, Systemic lupus erythematosus (SLE), Subacute bacterial endocarditis (SBE), Subacute cutaneous lupus, Susac syndrome, Sydenham's chorea, Sympathetic ophthalmia, Takayasu's arteritis (vasculitis), Testicular autoimmunity (vasculitis, orchitis), Tolosa-Hunt syndrome, Transverse Myelitis (TM), Tubulointerstitial nephritis uveitis syndrome (TINU), Ulcerative colitis (UC), Undifferentiated connective tissue disease (UCTD), Uveitis|anterior/intermediate/posterior, Vasculitis, VEXAS Syndrome, Vitiligo, Vogt-Koyanagi-Harada syndrome (VKH), and combinations thereof.

In some embodiments, treatment comprises an improvement in a patient or patient population's symptoms (e.g., as described herein). In some cases, the improvement is in comparison to a baseline or threshold amount. In some cases, the baseline or threshold amount is a value normally considered to be within a normal (e.g., healthy) range. In some cases, the baseline or threshold amount is based on a value prior to treatment and a value after treatment (e.g., a patient's own baseline score prior to treatment or a patient population's baseline score prior to treatment).

In some cases, the improvement is measured after administration, e.g., within a treatment cycle. In some cases, the response is measured within a week, e.g., within 1, 2, 3, 4, 5, 6, or 7 days after administration. In some cases, the response is measured after the last administration of a first treatment cycle (e.g., as described herein). In some cases, the response is measured after the last administration of a second treatment cycle (e.g., as described herein). In some cases, the response is measured within 1, 2, 3, 4, 5, 6, 7, 8, 9, 10, or 12 months after administration, e.g., the last administration of a treatment cycle.

### 1. Lupus

In some cases, the autoimmune disorder is systemic lupus erythematosus (SLE). In some cases, the autoimmune disorder is lupus nephritis.

Systemic lupus erythematosus (SLE) is a chronic autoimmune disease characterized by autoantibody production, abnormal B lymphocyte function and a loss of immune tolerance of endogenous nuclear material, which leads to systemic autoimmunity that can lead to damage to various tissues and organs (Pisetsky 2001, Anders 2020).

SLE can lead to arthritis, kidney failure, heart and lung inflammation, central nervous system (CNS) changes, vasculitis, severe skin rash, and blood dyscrasias such as anemia, leukopenia, and thrombocytopenia. The manifestations of SLE vary from patient to patient, and it may take many years to render the proper diagnosis.

Initial therapy for SLE typically consists of glucocorticoids in combination with either mycophenolate mofetil (MMF) or cyclophosphamide. Reasonable alternatives for initial therapy include MMF in combination with either a calcineurin inhibitor (voclosporin or tacrolimus) or belimumab, or cyclophosphamide in combination with belimumab. Patients in whom initial therapy fails should be switched to alternative therapies which include the usage of intravenous cyclophosphamide and/or an anti-CD20 monoclonal antibody (mAb).

Lupus nephritis (LN) is a form of glomerulonephritis and constitutes one of the most severe organ manifestations of SLE and in many cases, LN is the presenting manifestation that results in the diagnosis of SLE (Singh S 2009, Pons-Estel GL 2011). The goals of LN treatment are to achieve rapid remission of active disease, prevent renal flares, prevent progression of CKD, reduce morbidity and mortality, minimize treatment-associated toxicity, and preserve fertility. Standard of care treatment involves the use of immunosuppressive drugs, such as glucocorticoids, and cyclophosphamide (CYC) or mycophenolate mofetil (MMF) and adjuvant therapies and is based on histological disease classification. Immunosuppressive therapy is mostly used for treatment of class III and class IV LN and is divided into two phases: an induction phase with intensive immunosuppression, usually lasting 3-6 months; and a maintenance phase of prolonged, less intensive treatment to prevent renal flares.

However, conventional immunosuppressive treatments are not uniformly effective. The importance of achieving a complete or partial response was illustrated by the Lupus Nephritis Collaborative Study Group. Patients who achieved complete remission had better 10-year patient and renal survival rates (95% and 94%, respectively) as compared to those who attained partial remission (76% and 45%, respectively) (Korbert S M 2000). For patients who had no response to therapy, the 10-year patient and survival rate was 46% and 13% respectively (Chen Y E 2008) (Houssiau F A 2004. Houssiau F and Ginzler, 2008).

The management of patients with refractory disease varies with the first line agent used for induction therapy, clinical factors, and local practices. Switching to another first line induction agent is the recommended initial approach for patients with refractory LN and is recommended by both the EULAR/ERA-EDTA and American College of Rheumatology guidelines (Yo J H 2019). In general, CYC-resistant patients are treated with MMF and MMF-resistant patients with CYC. In addition to switching immunosuppressive agents, some accompany this with 3 days of intravenous pulses of glucocorticoids (Hahn B H 2012).

LN is histologically classified into six distinct classes that represent different manifestations and severities of renal involvement in SLE (Bajema I M 2018). The rationale for classifying LN into different classes is based on differences in the prognosis. Whereas class VI LN essentially represents kidney atrophy in end-stage kidney disease (ESKD), patients with class III, IV or V LN, but not class I or II LN, are immediate risk of chronic kidney disease (CKD) progression that reduces kidney lifespan (Romagnani P 2017, Mackay M 2019). Understanding of the genetic and pathogenetic basis of LN has improved substantially over the past few decades. However, despite this increased knowledge and improved treatment options, LN remains a substantial cause of morbidity and death among patients with SLE. Five-year mortality for LN decreased between 1975 and 1995 but remained stable thereafter, and the rate of progression to end-stage kidney disease (ESKD) remains unchanged (Croca S C 2011, Anders 2020).

B cells play a principal role in the pathogenesis of LN and are therefore attractive therapeutic targets. Rituximab (RTX) is a chimeric mouse-human monoclonal antibody directed against the B-cell surface molecule CD20. Several uncontrolled studies reported efficacy of RTX in patients with refractory LN (Lan 2012). However, RTX failed to show benefit in the EXPLORER trial which excluded patients with severe active LN (Merril J Y 2010). Additionally, the randomized, double-blind placebo-controlled trial (LUNAR) comparing RTX with placebo when added to standard of care with prednisolone and MMF in incident LN, also failed to demonstrate significant therapeutic efficacy (Rovin B H 2012). However, a higher complete response rate was seen with respect to proteinuria in patients who received RTX (32 vs 9%). This reduction in proteinuria persisted through to 78 weeks, raising the possibility that a longer period of follow up may have yielded a significant difference. The study also found higher partial response rates in the RTX group for all patients and in the prespecified subgroup of African-American patients, however, the trial was not powered to detect differences in partial response rates. (Yo J H 2019). A recent post hoc analysis of the LUNAR trial indicated that there was substantial variability in peripheral blood B cell depletion in patients with LN treated with RTX. Achievement of complete peripheral depletion (peripheral B cells 0 cells/μL) as well as the rapidity and duration of complete peripheral depletion were associated with complete response at week 78 (Gomez Mendez L M 2018). Reasons for this have been hypothesized to include autoreactive pathogenic B cells persisting within lymphoid structures and the kidney tubulointerstitium (Ahuja A 2007).

Despite the failure of the LUNAR trial to meet its primary endpoint, advocacy for the use of RTX in refractory LN continues and is primarily based on observational evidence, especially in patients who have an inadequate response to first line induction regimens. In a systematic review of published case reports and case series on the efficacy of RTX in patients with refractory LN, 300 patients who were followed up for 60 weeks had sustained complete and partial response rates in 87, 76, 67 and 76% of patients with class III, IV, V and mixed class respectively (Weidenbusch M 2013).

Obinutuzumab is an anti-CD20 monoclonal antibody that has been glycoengineered to increase antibody-dependent cellular cytotoxicity (ADCC). It has a type II binding conformation which leads to a greater direct cell death effect and more limited internalization of the monoclonal antibody. These characteristics result in a much more pronounced and sustained B cell depletion compared to rituximab [Reddy V 2017]. Since better B cell depletion, especially in the kidneys themselves, may increase the rate of CRR, obinutuzumab was tested in a small phase II trial, called NOBILITY [Furie 2020]. Obinutuzumab was compared to PBO on a MMF background and a moderate dose of promptly tapered glucocorticoids. The percentage of patients achieving complete renal response (CRR) was higher for patients given obinutuzumab compared to those receiving placebo, reaching 35% (versus 23%) at week 76 and 41% (versus 23%) at week 104. Almost all patients still had very low peripheral B cell counts (CD19+ count ≤5 cells/uL) at week 52, a finding much different from that of the LUNAR trial where only half of RTX-treated patients had undetectable peripheral B cells after one year of treatment [Rovin B H 2012]. Confirmation might come from a global phase III trial (NCT04221477).

The contribution of pathogenic B and T cells has been well studied in SLE but the role of innate immune cells has been less well established. Natural killer (NK) cells are key components of the innate immune system, providing immune surveillance by recognizing healthy cells and eliminating stressed cells, including virus-infected and cancer cells.

In SLE, NK cell dysfunction has been reported. NK cells are reduced in number in peripheral blood of SLE patients, have decreased cytotoxic function, impaired differentiation and altered cytokine production (Humbel, 2021, Liu, 2021, Lu, 2022). Interestingly, the reduction in NK cell number correlated with disease activity, where patients with higher disease activity had a greater reduction in NK cell number (Humbel, 2021). The dysfunction of NK cells in SLE may contribute to the pathogenesis of the disease by impairing the clearance of apoptotic cells and immune complexes, leading to the release of self-antigens and the activation of autoreactive B and T cells. Moreover, the altered cytokine production by NK cells may contribute to the chronic inflammation observed in SLE.

In addition to NK cell number reduction and dysfunction, there are reports that certain subsets of peripheral SLE NK cells demonstrate increased IFNγ production and an activated phenotype (Liu, 2021). However, the data are inconsistent across studies and may be related to the stage of the disease and treatment medication. Further, most knowledge on the characterization of NK cells in SLE has come from peripheral blood; less is known about the function of tissue resident NK cells. The kidney immune cell profile of SLE patients was recently evaluated by single-cell RNA sequencing where two distinct clusters of NK cells were identified (Azari, 2019). Additional studies are needed to determine how specific NK cell subsets contribute to the pathogenesis of SLE.

In some cases, treating SLE comprises an improvement (e.g., as described above) in Overall Renal Response Rate (ORR). In some cases, the improvement is as compared to the patient's ORR before treatment. In some cases, the improvement is as compared to a baseline or threshold amount. In some cases, the baseline or threshold amount is a value normally considered to be within a normal (e.g., healthy) range. In some cases, the baseline or threshold amount is based on the patient's own levels, e.g., prior to treatment. In some cases, the improvement is a Complete Renal Response (CRR). In some cases, CRR is a urinary protein-to-creatinine ratio (UPCR) of <0.5 and/or normal renal function (serum creatinine less than or equal to ULN) without worsening of baseline serum creatinine by more than 15%. In some cases, the improvement is a Partial Renal Response (PRR). In some cases, PRR is a 50% or more reduction in UPCR from baseline to a value<1 (to <3 if baseline UPCR was 3 or more) and/or serum creatinine not increased >15% from baseline. In some cases, the response is measured after administration, e.g., within a treatment cycle. In some cases, the response is measured within a week, e.g., within 1, 2, 3, 4, 5, 6, or 7 days after administration. In some cases, the response is measured after the last administration of a first treatment cycle (e.g., as described herein). In some cases, the response is measured after the last administration of a second treatment cycle (e.g., as described herein). In some cases, the response is measured within 1, 2, 3, 4, 5, 6, 7, 8, 9, 10, or 12 months after administration, e.g., the last administration of a treatment cycle. In some cases, the response (e.g., CRR or PERR) is measured at week 12, 22, 52, 76, and/or 104.

In some cases, treating SLE comprises an improvement (e.g., as described above) in one or more of: the SLE Disease Activity Index (SLEDAI), the Hybrid SELENA-SLEDAI (Systemic Lupus Erythematosus Disease Activity Index), the Systemic Lupus Activity Measure (SLAM), C3 complement levels, and C4 complement levels. In some cases, the improvement is at week 1, 2, 3, 4, 5, 6, 7, 8, 9, 10, 11, 12, 13, 14, 15, 16, 17, 18, 19, 20, 21, 22, 23, or 24 of a treatment cycle.

### 2. Rheumatoid Arthritis (RA)

In some cases, the autoimmune disorder is Rheumatoid arthritis (RA).

RA is a systemic, autoimmune disease with an annual incidence of 41/100,000 population in the US and Northem European countries (Myasoedova. 2021). It is characterized by a symmetrical inflammatory polvarthritis, but extra-articular features are common and are often associated with poor prognosis. Refractory RA has emerged as an area of unmet need. Most patients are adequately managed on methotrexate and other first-line disease-modifying anti-rheumatic drugs (DMARDs e.g., sulfasalazine, hydroxychloroquine, leflunomide), a proportion of patients require a biologic disease-modifying anti-rheumatic drugs (bDMARD e.g., Infliximab, Rituximab, Etanercept, Tocilizumab) and/or a targeted synthetic disease-modifying anti-rheumatic drugs (tsDMARD e.g., baricitinib, tofacitinib), with a further subsection failing multiple agents. Recent observational studies have adopted working definitions of refractory RA based on the number of failed DMARDs, with prevalence estimates of refractory RA ranging from 6-21% of all patients treated with conventional DMARDs (Melville, 2020).

In some cases, treating RA comprises an improvement (e.g., as described above) in one or more of: the rheumatoid arthritis disease activity score DAS28, the clinical disease activity index (CDAI), and participant response assessed by EULAR criteria. In some cases, the improvement is at week 1, 2, 3, 4, 5, 6, 7, 8, 9, 10, 11, 12, 13, 14, 15, 16, 17, 18, 19, 20 21, 22, 23, or 24 of a treatment cycle.

### 3. Pemphigus Vulgaris (PV)

In some cases, the autoimmune disorder is pemphigus vulgaris (PV).

Pemphigus is a group of autoimmune bullous disorders involving mucous membranes and/or skin. The most common type of pemphigus is pemphigus vulgaris (PV), which accounts for approximately 80% of pemphigus. Pemphigus vulgaris is an autoimmune skin disease caused by cadherin desmoglein (Dsg) specific autoantibodies that bind to epidermal desmosomes, resulting in blisters and erosions of the mucous membranes or skin. Although various modalities have been tried, the disease has been a challenge to manage. Targeting pathogenic immune pathways with anti-CD20 monoclonal antibodies, BAFF inhibitors, IL-17 blockade, mTOR pathway inhibitors, p38 MAPK, BTK inhibitors and TNF-α inhibitors have all been tried in the management of PV (Abulikemu, 2023). However, the disease continues to be challenging to manage with patients having flares and developing resistance or intolerance to existing treatments.

In some cases, treating PV comprises an improvement (e.g., as described above) in one or more of: the Pemphigus Disease Area Index (PDAI), time to disease flares, number of disease flares, time to initial complete remission evaluated by PDAI, change in health related QoL by Dermatology Life Quality Index (DLQI), and blood DSG 1 and 3 levels. In some cases, the improvement is at week 1, 2, 3, 4, 5, 6, 7, 8, 9, 10, 11, 12, 13, 14, 15, 16, 17, 18, 19, 20 21, 22, 23, or 24 of a treatment cycle.

4. Granulomatosis with Polyangiitis (GPA) and Microscopic Polyangiitis (MPA)

In some cases, the autoimmune disorder is granulomatosis with polyangiitis (GPA) and/or microscopic polyangiitis (MPA).

Granulomatosis with polyangiitis (GPA) and microscopic polyangiitis (MPA) are forms of small-medium vessel vasculitis. GPA and MPA are rare diseases with a prevalence of 24 to 160 patients per million and 39 to 94 per million, respectively. Although no validated diagnostic criteria exist, the 1990 American College of Rheumatology (ACR) classification criteria (for GPA) and the 2012 Chapel Hill Consensus Conference nomenclature help define these diseases for the purposes of clinical trials, as their clinical presentation makes them amenable to innovative therapeutic approaches. Both GPA and MPA commonly cause pulmonary and/or renal manifestations, with GPA frequently affecting the upper airway. Because of their clinical similarities, GPA and MPA are frequently studied together in clinical trials. Multidisciplinary approaches are needed for the diagnosis and management of vasculitis because of its systemic nature (Koike, 2022).

In some cases, treating GPA and/or MPA comprises an improvement (e.g., as described above) in one or more of the Birmingham Vasculitis Activity Score, time to disease flares, number of disease flares, time to initial complete remission, change in health related QoL by Dermatology Life Quality Index (DLQI). In some cases, the improvement is at week 1, 2, 3, 4, 5, 6, 7, 8, 9, 10, 11, 12, 13, 14, 15, 16, 17, 18, 19, 20 21, 22, 23, or 24 of a treatment cycle.

### B. Patients

Suitable patients for the compositions and methods herein include those who are suffering from, who have been diagnosed with, or who are suspected of having an autoimmune disorder, e.g., as described herein.

In some embodiments, the methods of treatment provided herein may be used to treat a subject (e.g., human, monkey, dog, cat, mouse) who has been diagnosed with or is suspected of having an autoimmune disorder, e.g., as described herein. In some embodiments, the subject is a mammal. In some embodiments, the subject is a human.

In some embodiments, the patient is or has been diagnosed with an autoimmune disorder, e.g., and autoimmune disorder described herein. In some cases, the patient is resistant to, relapsed, or refractory after initial treatment for the disorder. In some cases, the patient has failed at least two lines of previous treatments in alignment with the Standard of Care (SOC).

In some cases, the patient has systemic lupus erythematosus (SLE).

In some cases, the patient's SLE is diagnosed according to the 2010 American College of Rheumatology (ACR)/European League Against Rheumatism (EULAR) classification criteria for RA (Kay, 2012).

In some cases, the patient has a total systemic lupus erythematosus disease activity index (SLEDAI-2K) total score of six or more prior to treatment. In some cases, the patient has a SLEDAI-2K total score of eight or more prior to treatment, excluding alopecia, mucosal ulcers, and fever.

In some cases, the patient has tried and failed conventional SLE therapies (e.g., at least 12 weeks of 2 conventional therapies), including, for example: antimalarials, corticosteroids, immunosuppressive agents such as mycophenolate mofetil. Methotrexate, Azathioprine, and/or biological agents such as Belimumab, Anifrolumab. and Rituximab. In some cases, the patient has received antimalarial drugs (e.g., hydrochloroquine, chloroquine, quinacrine), e.g. for 12 or more weeks prior to the first treatment and optionally at a stable dose for a minimum of 6 weeks prior to the first administration of NK cells. In some cases, the patient has received immunomodulatory drugs (e.g., mycophenolate mofetil (MMF)/mycophenolic acid, azathioprine/6 mercaptopurine, leflunomide, methotrexate with or without concomitant folic acid, calcineurin inhibitor, and/or cyclosporin A), e.g. at a stable dose for at least 12 weeks prior to the first administration of NK cells.

In some cases, the patient has lupus nephritis. In some cases, the patient has Class I, Class II, Class III, Class IV, Class V, and/or Class VI lupus nephritis. In some cases, the patient has minimal mesangial lupus nephritis, mesangial proliferative lupus nephritis, focallupus nephritis, diffuse lupus nephritis, membranous lupus nephritis, and/or advanced sclerosis lupus nephritis. In some cases, the lupus nephritis is refractory, e.g., the patient has failed to improve after 3-4 months of prior treatment, has not achieved partial response after 6-12 months, and/or has not achieved complete response after 2 years of prior treatment.

In some cases, the patient has evidence of active disease on a renal biopsy based on the modified NIH Lupus Nephritis activity and chronicity indices (Bajema I M 2018). In some cases, the patient has detectable anti-double stranded DNA antibody titers.

In some cases, the patient is a subject (e.g., an adult subject) with lupus nephritis Class III or IV, either with or without the presence of Class V according to the 2018 ISN/RPS criteria (Bajema I M 2018). In some cases, the patient is relapsed or refractory after initial therapy for SLE. In some cases, the initial therapy comprises one or more of, glucocorticoids in combination with either mycophenolate mofetil (MMF) or cyclophosphamide, MMF in combination with either a calcineurin inhibitor (e.g., voclosporin or tacrolimus) or belimumab, cyclophosphamide in combination with belimumab, intravenous cyclophosphamide, an anti-CD20 monoclonal antibody (mAb), or intravenous cyclophosphamide in combination with an anti-CD20 monoclonal antibody (mAb). In some cases, the patient has failed to improve within 3-4 months of a previous treatment, has not achieved partial response after 6-12 months of a previous treatment, or has not achieved complete response after 2 years of a previous treatment. In some cases, the patient has failed at least two lines of previous treatments in alignment with the Standard of Care (SOC) for subjects with lupus nephritis.

In some cases, the patient has rheumatoid arthritis (RA).

In some cases, the patient's RA is diagnosed according to the 2010 American College of Rheumatology (ACR)/European League Against Rheumatism (EULAR) classification criteria for RA (Kay, 2012). In some cases, the patient has had prior treatment with a biologic disease-modifying anti-rheumatic drug (a “bDMARD,” e.g., infliximab, rituximab, etanercept, tocilizumab) and/or a targeted synthetic disease-modifying anti-rheumatic drug (a “tsDMARD,” e.g., baricitinib, tofacitinib) and were deemed refractory, e.g., by one or more of: lack of benefit to a bDMRD or a tsDMARD: lack of benefit to at least two bDMRDs; lack of benefit to at least one bDMARD and one tsDMARD: intolerance to one or more lines of prior therapy (e.g., one, two, or three lines of prior therapy), including bDMARDs and/or tsDMARDs. In some cases, lack of benefit includes inadequate improvement in one or more of: joint counts, physical function, and disease activity. In some cases, the patient has a minimum of 1, 2, 3, 4, 5, 6, 7, 8, 9, or 10 swollen joint counts (SJC) and/or a minimum of 1, 2, 3, 4, 5, 6, 7, 8, 9, or 10 tender joint counts (TJC). In some cases, the patient has a minimum of 6 swollen joint counts (SJC) and 6 tender joint counts (TJC).

In some cases, the patient has pemphigus vulgaris (PV).

In some cases, the patient has one or more of active lesions; positive for anti-desmoglein Dsg1 or Dsg3; and a pemphigus disease area index core (Shimizu, 2014) of >80%.

In some cases, the patient has granulomatosis with polyangiitis (GPA) and/or microscopic polyangiitis (MPA). In some cases, the patient has one or more of: ≥1 “major” item, ≥3 “other” items, and ≥2 renal items on the Birmingham Vasculitis Activity Score Version 3 (BVASv3).

### C. Lymphodepletion

In some embodiments, the patient is lymphodepleted before treatment.

Illustrative lymphodepleting chemotherapy regimens, along with correlative beneficial biomarkers, are described in WO 2016/191756 and WO 2019/079564, hereby incorporated by reference in their entirety. In certain embodiments, the lymphodepleting chemotherapy regimen comprises administering to the patient doses of cyclophosphamide (between 200 mg/m²/day and 2000 mg/m²/day) and doses of fludarabine (between 20 mg/m²/day and 900 mg/m²/day).

In some embodiments, lymphodepletion comprises administration of or of about 100 to or to about 1500 mg/m² of cyclophosphamide, e.g., of or of about 250 to or to about 500 mg/m² of cyclophosphamide, e.g., from or from about 250 to or to about 500, 250, 400, 500, about 250, about 400, or about 500 mg/m² of cyclophosphamide. In some embodiments, lymphodepletion comprises administration of or of about 500 mg/m² of cyclophosphamide. In some embodiments, lymphodepletion comprises administration of or of about 100 mg/m² of cyclophosphamide.

In some embodiments, lymphodepletion comprises administration of or of about 20 mg/m²/day to or to about 40 mg/m²/day fludarabine. e.g., 30 or about 30 mg/m²/day.

In some embodiments, lymphodepletion comprises administration of both cyclophosmamide and fludarabine.

In some embodiments, the patient is lymphodepleted by intravenous administration of cyclophosphamide (250 mg/m²/day) and fludarabine (30 mg/m²/day).

In some embodiments, the patient is lymphodepleted by intravenous administration of cyclophosphamide (500 mg/m²/day) and fludarabine (30 mg/m²/day).

In some embodiments, the lymphodepletion occurs no more than 5 days prior to the first dose of NK cells. In some embodiments, the lymphodepletion occurs no more than 7 days prior to the first dose of NK cells.

In some embodiments, lymphodepletion occurs daily for 3 consecutive days, starting 5 days before the first dose of NK cells.

In some embodiments, the first dose of NK cells is given on day 6 and lymphodepletion occurs on days 1, 2, and 3.

### D. Administration

Described herein are methods comprising administration of NK cells (e.g., as described herein). In some cases, the NK cells are administered as part of a therapy further comprising administration of one or more additional agents, including, for example, an antibody (e.g., as described herein), a cytokine (e.g., as described herein), a lymphodepleting agent (e.g., as described herein), a corticosteroid (e.g., prednisone, prednisolone, dexamethasone, methylprednisolone), an analgesic, an antipyretic, and/or an antihistamine.

For example, patients may be pre-treated/pre-medicated prior to NK cell infusion, e.g., as described herein. For example, as set forth in Table 13 or Table 14.

### 1. NK Cells

In some embodiments, the NK cells are administered as part of a pharmaceutical composition, e.g., a pharmaceutical composition described herein. Cells are administered after thawing, in some cases without any further manipulation in cases where their cryoprotectant is compatible for immediate administration. For a given individual, a treatment regimen often comprises administration over time of multiple aliquots or doses of NK cells, which can be drawn from a common batch or donor. In some embodiments, the NK cells, e.g., the NK cells described herein are administered at or at about 1×10⁸ to or to about 8×10⁹ NK cells per dose. In some embodiments, the NK cells are administered at or at about 1×10⁸, at or at about 1×10⁹, at or at about 4×10⁹, or at or at about 8×10⁹ NK cells per dose. In some cases, the NK cells are administered at or about 4 billion cells per dose. In some cases, the NK cells are administered at or about 2 billion cells per dose.

In some embodiments, the NK cells are administered weekly. In some embodiments, the NK cells are administered for or for about 3-8 weeks. In some embodiments, the NK cells are administered for or for about 4-8 weeks. In some embodiments, the NK cells are administered weekly for or for about 4 weeks. In some embodiments, the NK cells are administered weekly for or for about 8 weeks.

In some embodiments, the NK cells are administered on days 6, 9, 13, and 16 of a treatment cycle. In some embodiments, the NK cells are administered on days 6, 13, and 20 of a treatment cycle.

In some embodiments, administration is repeated for one or more additional treatment cycles, e.g., following an administration scheduled described above. In some embodiments, the additional treatment cycle is or is about 2-12 months after the previous treatment cycle, e.g., 2, 3, 4, 5, 6, 7, 8, 9, 10, 11, or 12 months, or thereabout, after the previous treatment cycle.

In some embodiments, the NK cells are cryopreserved in an infusion-ready media, e.g., a cryopreservation composition suitable for intravenous administration, e.g., as described herein.

In some embodiments, the NK cells are cryopreserved in vials containing from or from about 1×10⁸ to or to about 8×10⁹ cells per vial. In some embodiments, the NK cells are cryopreserved in vials containing a single dose.

In some embodiments, the cells are thawed, e.g., in a 37° C. water bath, prior to administration.

In some embodiments, the thawed vial(s) of NK cells are aseptically transferred to a single administration vessel, e.g., administration bag using, e.g., a vial adapter and a sterile syringe. The NK cells can be administered to the patient from the vessel through a Y-type blood/solution set filter as an IV infusion, by gravity.

In some embodiments, the NK cells are administered as soon as practical, preferably less than 90 minutes, e.g., less than 80, 70, 60, 50, 40, 30, 20, or 10 minutes after thawing. In some embodiments, the NK cells are administered within 30 minutes of thawing.

In some embodiments, the pharmaceutical composition is administered intravenously via syringe.

In some embodiments, 1 mL. 4 mL, or 10 mL of drug product is administered to the patient intravenously via syringe.

### 2. Antibodies

In some embodiments, the NK cell(s) described herein, e.g., the pharmaceutical compositions comprising NK cell(s) described herein, are administered in combination with an antibody or antibodies. e.g., an antibody or antibodies described herein. e.g., a B-cell depleting antibody. e.g., a CD19 and/or CD20 antibody. e.g., Rituximab and/or Obinutuzumab. In some embodiments, the antibodie(s) are administered together with the NK cells as part of a pharmaceutical composition. In some embodiments, the antibodie(s) are administered separately from the NK cells, e.g., as part of a separate pharmaceutical composition. Antibodies can be administered prior to, subsequent to, or simultaneously with administration of the NK cells.

In some embodiments, the antibody is administered before the NK cells. In some embodiments, the antibody is administered after the NK cells.

In some embodiments, the NK cells are administered at least 30 minutes, 60 minutes, 90 minutes. 120 minutes. 150 minutes. 180 minutes. 210 minutes, or 240 minutes after completing administration of the antibody.

In some embodiments, the NK cells are administered the day after the antibody is administered.

In some embodiments, the NK cells are administered at each administration, while the antibody is administered at a subset of the administrations. For example, in some embodiments, the NK cells are administered once a week and the antibody is administered once a month.

In some embodiments, the antibody is administered weekly for 8 weeks. In some embodiments, the antibody is administered every two weeks for 8 weeks.

In some embodiments, a dose of antibody is given prior to the first dose of cells. In some embodiments, a debulking dose of the antibody is given prior to the first dose of cells.

In some embodiments, the antibody is administered on days 2 and 13 of a treatment cycle. In some embodiments, the antibody is administered on days 1 and 15 of a treatment cycle.

### 3. Cytokines

In some embodiments, a cytokine is administered to the patient.

In some embodiments, the cytokine is administered together with the NK cells as part of a pharmaceutical composition. In some embodiments, the cytokine is administered separately from the NK cells, e.g., as part of a separate pharmaceutical composition. In some embodiments, the cytokine is IL-2.

In some embodiments, a cytokine is not administered to the patient.

### E. Dosing

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography, flow cytometry, or a molecular assay.

### F. Treatment Cycles

In some cases, treatment comprises administration of the NK cells and antibodies (e.g., as described herein), over the course of a treatment cycle. For example, in some cases, a treatment cycle comprises lymphodepletion followed by administrations of NK cells and a B-cell depleting antibody. In some cases, the B-cell depleting antibody is administered prior to the first administration of NK cells and. In some cases, the B-cell depleting antibody is administered both prior to the first administration of NK cells and after the first administration of NK cells (e.g., spaced about two weeks apart, e.g., at days 2 and 13). In some cases, the NK cells are administered one or more times (e.g., 1, 2, 3, 4, 5, 6, 7, 8, 9, or 10 times) during the treatment cycle, e.g., weekly. In some cases, the NK cells are administered about weekly after lymphodepletion (e.g., on days 6, 13, 20, and so on). In some cases, the NK cells are administered on days 6, 13, and 20. In some cases, the treatment cycle is repeated one or more times, e.g., 1, 2, 3, 4, 5, or 6 times. In some cases, the treatment cycles are spaced about one or more months apart, e.g., about 1, 2, 3, 4, 5, 6, 7, 8, 9, 10, 11, or 12 months apart.

For example, an exemplary treatment cycle (e.g., for SLE) comprises lymphodepletion at the beginning of the treatment cycle (e.g., before administration of NK cells). In some cases, lymphodepletion comprises 3 consecutive days (Days 1-3) of a lymphodepleting regimen to induce lymphocyte depletion and to create an optimal environment for expansion of NK cells in vivo. In this example, a B-cell depleting antibody (e.g., Rituximab or Obinutuzumab) is administered twice (spaced ~2 weeks apart) as an intravenous infusion (e.g., of 1000 mg) during the treatment cycle, optionally after premedication with methylprednisolone to reduce the risk of infusion-related reactions. In some cases, NK cells are dosed after the administration of the lymphodepleting regimen and after the first dose of antibody, by IV infusion. In some cases, the treatment cycle comprises dosing NK cells according to one of the dosing levels in Table 4. The lymphodepletion regimen can comprise, for example, a fludarabine and cyclophosphamide regimen, e.g., in which fludarabine is administered at 30 mg/m² on day 1, 2, and 3 at the beginning of each treatment cycle and cyclophosphamide is administered at 1000 mg/m² on day 3 of each treatment cycle. In one example, the antibody is administered at 1000 mg on Day 2 and Day 13 of each treatment cycle. In some cases, the first dose of AB-101 is administered at least 48 hours after the last infusion of the lymphodepletion regimen for each treatment cycle, using the dosing scheme set forth in Table 4. In another example, a second treatment cycle is administered, e.g., approximately 24 weeks after the first dose of NK cells in the first treatment cycle.

TABLE 4
Example Dose Levels and Dosing Days for AB-101

| Dosing | Day 6 | Day 9 | Day 13 | Day 16 | Day 20 |
|---|---|---|---|---|---|
| Dose Level −1 | 2 × 10⁹ cells | NA | 2 × 10⁹ cells | NA | 1 × 10⁹ cells |
| Dose Level 1 | 4 × 10⁹ cells | NA | 4 × 10⁹ cells | NA | 2 × 10⁹ cells |
| Dose Level 2 | 4 × 10⁹ cells | 2 × 10⁹ cells | 4 × 10⁹ cells | 2 × 10⁹ cells | NA |

In another example, a treatment cycle (e.g., for SLE) comprises lymphodepletion at the beginning of the treatment cycle (e.g., before administration of NK cells). In this example, lymphodepletion comprises 3 consecutive days (Days 1-3) of a lymphodepleting regiment and a B-cell depleting antibody (e.g., Rituximab or Obinutuzumab) is administered twice (spaced ~2 weeks apart, e.g., on days 2 and 13) as an intravenous infusion (e.g., of 1000 mg) during the treatment cycle, optionally after premedication to reduce the risk of infusion-related reactions (e.g., as described herein). In some cases, NK cells are dosed after the administration of the lymphodepleting regimen and after the first dose of antibody, by IV infusion. In some cases, the treatment cycle comprises dosing NK cells according to one of the dosing levels in Table 5. The lymphodepletion regimen can comprise, for example, a fludarabine and cyclophosphamide regimen, e.g., in which fludarabine is administered at 30 mg/m² on day 1, 2, and 3 at the beginning of each treatment cycle and cyclophosphamide is administered at 1000 mg/m² on day 3 of each treatment cycle. In one example, the antibody is administered at 1000 mg on Day 2 and Day 13 of each treatment cycle. In some cases, the first dose of AB-101 is administered at least 48 hours after the last infusion of the lymphodepletion regimen for each treatment cycle, using the dosing scheme set forth in Table 5. In some cases, AB-101 is administered on days 6, 13, and 20 of the treatment cycle. In some cases, a second treatment cycle is administered, e.g., approximately 24 weeks after the first dose of NK cells in the first treatment cycle.

TABLE 5
Example Dose Levels for AB-101 and B-cell Depleting Antibody

| Cohorts | Paired mAb | AB-101 Dose Levels |
|---|---|---|
| Monotherapy DL−1 | None | Dose Level −1 (5 × 10⁸ cells) |
| Monotherapy DL1 | None | Dose Level 1: 1 × 10⁹ cells |
| Monotherapy DL2# | None | Dose Level 1: 4 × 10⁹ cells |
| Rituxmab DL1 | Rituximab 1,000 mg/dose | Dose Level 1: 1 × 10⁹ cells |
| Rituximab DL2 | Rituximab 1,000 mg/dose | Dose Level 1: 4 × 10⁹ cells |
| Obinutuzumab DL1 | Obinutuzumab 1,000 mg/dose | Dose Level 1: 1 × 10⁹ cells |
| Obinutuzumab DL2 | Obinutuzumab 1,000 mg/dose | Dose Level 1: 4 × 10⁹ cells |

### V. VARIANTS

In some embodiments, the fusion protein(s) or components thereof described herein, or the NK cell genotypes described herein, are at least 80%, e.g., at least 85%, 90%, 95%, 98%, or 100% identical to the amino acid sequence of an exemplary sequence (e.g., as provided herein), e.g., have differences at up to 1%. 2%, 5%, 10%, 15%, or 20% of the residues of the exemplary sequence replaced, e.g., with conservative mutations, e.g., including or in addition to the mutations described herein. In preferred embodiments, the variant retains desired activity of the parent.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e. a query) and a second polypeptide or nucleic acid sequence (i.e. target) is determined in various ways that are within the skill in the art, for instance, using publicly available computer software such as Smith Waterman Alignment (Smith. T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool: (Altschul, S. F., W. Gish, et al. (1990) J Mol Biol 215: 403-10), BLAST-2, BLAST-P, BLAST-N, BLAST-X, WU-BLAST-2, ALIGN. ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

### VI. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example. 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating.” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes place contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the term “buffer solution” refers to an aqueous solution consisting of a mixture of a weak acid and its conjugate base, or vice versa.

As used herein, the term “cell culture medium” refers to a mixture for growth and proliferation of cells in vitro, which contains essential elements for growth and proliferation of cells such as sugars, amino acids, various nutrients, inorganic substances, etc.

A buffer solution, as used herein, is not a cell culture medium.

As used herein, the term “bioreactor” refers to a culture apparatus capable of continuously controlling a series of conditions that affect cell culture, such as dissolved oxygen concentration, dissolved carbon dioxide concentration, pH, and temperature.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule(s) or polynucleotide(s) of the present application. Certain vectors are capable of directing the expression of nucleic acids to which they are operatively linked. Such vectors are referred to herein as expression vectors.

The term “operably linked” refers to two or more nucleic acid sequence or polypeptide elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The terms “host cell,” “host cell line,” and “host cell culture” are used interchangeably and refer to cells into which exogenous nucleic acid has been introduced, including the progeny of such cells. Host cells include “engineered cells,” “transformants.” and “transformed cells,” which include the primary engineered (e.g., transformed) cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with a polynucleotide encoding a fusion protein, as described herein.

The section headings used herein are for organizational purposes only and are not to be construed as limiting the subject matter described.

### VII. REFERENCES

Ahuja A, Shupe J, Dunn R, Kashgarian M, Kehry M R, Shlomchik M J. Depletion of B cells in murine lupus: efficacy and resistance. J Immunol. 2007:179(5):3351
ACTEMERA® Prescribing Information. Genentech USA. Inc.; 2017
Albert D, Dunham J, Khan S, Stansberry J, Kolasinski S, Tsai D, Pullman-Mooar S, Bamack F, Striebich C, Looney R J, Prak E T, Kimberly R, Zhang Y, Eisenberg R.
Variability in the biological response to anti-CD20 B cell depletion in systemic lupus ervthaematosus. Ann Rheum Dis. 2008 December: 67(12):1724-31. doi: 10.1136/ard.2007.083162. Epub 2008 Feb. 4. PMID: 18250115.
Anders H J, Saxena R, Zhao M H, et al., Lupus nephritis. Nat Rev Dis Primers. 2020; 23; 6(1); 7
Arazi A, Rao D A, Berthier C C, et al. The immune cell landscape in kidneys of patients with lupus nephritis [published correction appears in Nat Immunol. 2019 Aug. 13]. Nat Immunol. 2019; 20(7):902-914. doi:10.1038/s41590-019-0398-x
Bajema I M, Wilhelmus S, Alpers C E, Bruijn J A, Colvin R B, Cook H T. D'Agati V D. Ferrario F, Haas M, Jennette J C, Joh K, Nast C C, Noel L H, Rijnink E C, Roberts I SD, Seshan S V, Sethi S, Fogo A B. Revision of the International Society of Nephrology/Renal Pathology Society classification for lupus nephritis: clarification of definitions, and modified National Institutes of Health activity and chronicity indices. Kidney Int. 2018 April; 93(4):789-796
Boross P, Leusen J H. Mechanisms of action of CD20 antibodies. Am J Cancer Res. 2012:2(6):676-90. Epub 2012 Nov. 20. PMID: 23226614; PMCID: PMC3512181.
Chen Y E, Korbet S M, Katz R S et al. Value of a complete or partial remission in severe lupus nephritis. Clin Jam Soc Nephrol. 2008; 3(1); 46-53
Cooley S, Trachtenberg E, Bergemann T L et al. Donors with group B KIR haplotypes improve relapse-free survival after unrelated hematopoietic cell transplantation for acute myelogenous leukemia. Blood. 2009; 113(3): 726-732
Croca S C, Rodrigues T and Isenberg D A. Assessment of a lupus nephritis cohort over a 30 year period. Rheumatology. 2011; 50: 1424-1430
Depil S, Duchateau P, Grupp S A, Mufti G, Poirot L. ‘Off-the-shelf’ allogeneic CAR T cells: development and challenges. Nat Rev Drug Discov. 2020 March; 19(3):185-199. doi: 10.1038/s41573-019-0051-2. Epub 2020 Jan. 3. PMID: 31900462.
Desai A, Gupta R, Advani S. et al. Mortality in Hospitalized Patients With Cancer and Coronavirus Disease 2019: A Systematic Review and Meta-Analysis of Cohort Studies. Cancer. 2021; 127(9):1459-1468.
Dudley M E, Wunderlich J R, Yang J C. et al. Adoptive cell transfer therapy following non-myeloablative but lymphodepleting chemotherapy for the treatment of patients with refractory metastatic melanoma. Journal of clinical oncology: official journal of the American Society of Clinical Oncology. 2005 Apr. 1; 23(10):2346.
ENHERTU® (fam-trastuzumab deruxtecan-nxki). Prescribing information. Daiichi Sankyo, Inc.; 2022.
Freeman A J, Vervoort S J, Ramsbottom K M, et al. Natural Killer Cells Suppress T Cell-Associated Tumor Immune Evasion. Cell Rep. 2019; 28(11):2784-2794.
Furie, R.: Aroca, G.; Alvarez, A.; Fragoso-Loyo, H.; Zuta Santillan, E.: Rovin, B.; Brunetta, P.; Schindler, T.; Hassan, I.; Cascino, M.; et al. Two-year results from a randomized; controlled study of obinutuzumab for proliferative lupus nephritis (abstract). Arthritis Rheumatol. 2020, 72 (Suppl. 10).
Gattinoni, L, Finkelstein S E. Klebanoff C A. et al. Removal of homeostatic cytokine sinks by lymphodepletion enhances the efficacy of adoptively transferred tumor-specific CD8+ T cells. J Exp Med. 2005 Oct. 3; 202(7):907-12. doi: 10.1084/jem.20050732
Gladman D D, Ibanez D, Urowitz M B et al. Systemic lupus erythematosus disease activity index. 2000. J Rheumatol. 2002; 29(2): 288-291
Gomez Mendez L M, Cascino M D, Garg J, et al. Peripheral blood B cell depletion after rituximab and complete response in lupus nephritis. Clin J Am Soc Nephrol. 2018:13(10):1502-1509
Green, M R J et al. “Natural killer cell activity in families of patients with systemic lupus erythematosus: demonstration of a killing defect in patients.” Clinical and experimental immunology vol. 141,1 (2005): 165-73. doi:10.1111/j.1365-2249.2005.02822.x
Guillerey C, Huntington N D and Smyth M J. Targeting natural killer cells in cancer immunotherapy. Nat. Immunology. 2016; 17(9); 1025-1036
Hahn B H, McMahon M A, Wilkinson A et al. American College of Rheumatology guidelines for screening treatment and management of lupus nephritis. Arthritis Care Res (Hoboken). 2012:64(6): 797-808
Hanly J G, O'Keeffe A G, Su I, et al. The frequency and outcome of lupus nephritis: results from an international inception cohort study. Rheumatology. 2016; 55(2): 252-262
Harris A, Young R, Devine S, et al. International, Multicenter Standardization of Acute Graft-versus-Host Disease Clinical Data Collection: A Report from the Mount Sinai Acute GVHD International Consortium. Biol Blood Marrow Transplant 22 (2016) 4-10
Hartmann J, SchfiBler-Lenz M, Bondanza A, Buchholz C J. Clinical development of CAR T cells-challenges and opportunities in translating innovative treatment concepts. EMBO Mol Med. 2017; 9(9):1183-1197. doi:10.15252/emmm.201607485
Houssiau F A and Ginzler E M. Current treatment of lupus nephritis. Lupus. 2008: 17(5): 426-430.
Houssiau F A, Vasconcelos C, D'Cruz D et al. Early response to immunosuppressive therapy predicts good renal outcome in lupus nephritis: lessons from long-term follow-up of patients in the Euro-Lupus Nephritis Trial. Arthritis Rheum. 2004; 50(12): 3934-3940
Humbel M, Bellanger F, Fluder N, Horisberger A, Suffiotti M, Fenwick C, Ribi C, Comte D. Restoration of NK Cell Cytotoxic Function With Elotuzumab and Daratumumab Promotes Elimination of Circulating Plasma Cells in Patients With SLE. Front Immunol. 2021 Mar. 22; 12:645478. doi: 10.3389/fimmu.2021.645478. PMID: 33828555; PMCID: PMC8019934.
ICMJE 2021
Khanal R, Meht A, Maly J, et al. AB-101, an Allogeneic, Non-Genetically Modified, Natural Killer (NK) Cell Therapy, Evaluated as Monotherapy or in Combination with Rituximab in R/R Non-Hodgkin Lymphoma.
Klebanoff C A, Gattinoni L, Torabi-Parizi P, et al. Central memory self-tumor-reactive CD8+ T cells confer superior antitumor immunity compared with effector memory T cells. PNAS, Jul. 5, 2005, vol. 102, no. 27, 9571-9576
Korbert S M, Lewis El, Schwartz M M et al. Factors predictive of outcome in severe lupus nephritis. Lupus Nephritis Collaborative Study Group. 2000. Am J Kidney Dis; 35(5): 904-914
Kozlowski, S H et al. “Comparison of natural killing with antibody dependent cell mediated cytotoxicity in patients with systemic lupus erythematosus.” The Journal of rheumatology vol. 9.1 (1982): 59-62.
Lan et al./J Zhejiang Univ-Sci B (Biomed & Biotechnol) 2012 13(9):731-744
Le R Q, Li L, Yuan W, et al. FDA Approval Summary: Tocilizumab for Treatment of Chimeric Antigen Receptor T Cell-Induced Severe or Life-Threatening Cytokine Release Syndrome. The Oncologist, Vol. 23, Issue 8, August 2018, 943-947
Lee D S, Rojas O L, Gommerman J L. B cell depletion therapies in autoimmune disease: advances and mechanistic insights. Nat Rev Drug Discov 2021; 20:179-199
Lee D W, Santomasso B D, Locke F L, et al. ASTCT Consensus Grading for Cytokine Release Syndrome and Neurologic Toxicity Associated with Immune Effector Cells. Biol Blood Marrow Transplant 2019 25: 625-638.
Liu, Manman et al. “Activation status of CD56dim natural killer cells is associated with disease activity of patients with systemic lupus erythematosus.” Clinical rheumatology vol. 40.3 (2021): 1103-1112. doi: 10.1007/s10067-020-05306-x
Lu, Zhimin et al. “Increased oxidative stress contributes to impaired peripheral CD56dimCD57+NK cells from patients with systemic lupus erythematosus.” Arthritis research & therapy vol. 24,1 48, 16 Feb. 2022. doi:10.1186/s13075-022-02731-y
Mackensen A, Muller F, Mougiakakos D et al. Anti-CD19 CAR T cell therapy for refractory systemic lupus erythematosus. Nat Med. 2022; 28(10): 2124-2132
MacKay M, Dall'Era M. Fishbein J et al. Establishing Surrogate Kidney End Points for Lupus Nephritis Clinical Trials: Development and Validation of a Novel Approach to Predict Future Kidney Outcomes. Arthritis Rheumatol. 2019; 71(3): 411-419
Malmberg K J, Carlsten M. Bjorklund A et al. Natural killer cell-mediated immunosurveillance of human cancer. Semin Immunol. 2017 June: 31: 20-29. Doi:10.1016/j.smim.2017.08.002. Epub 2017 Sep. 6. PMID: 28888619.
Manches O, Lui G. Chaperot L, Gressin R, Molens J P, Jacob M C, Sotto J J, Leroux D, Bensa J C, Plumas J. In vitro mechanisms of action of rituximab on primary non-Hodgkin lymphomas. Blood. 2003 Feb. 1; 101(3):949-54. doi: 10.1182/blood-2002-02-0469. Epub 2002 Oct. 3. PMID: 12393572.
Merrill J T, Neuwelt C M, Wallace D J, et al. Efficacy and safety of rituximab in moderately-to-severely active systemic lupus erythematosus: the randomized, double-blind, phase II/III systemic lupus erythematosus evaluation of rituximab trial. Arthritis Rheum. 2010; 62(1):222-233. doi: 10.1002/art.27233
Miller J S. Therapeutic applications: natural killer cells in the clinic. Hematology Am Soc Hematol Educ Program. 2013; 2013:247-53
Moretta A, Locatelli F. Moretta L. Human NK cells: from HLA class I-specific killer Ig-like receptors to the therapy of acute leukemias. Immunological reviews. 2008 August; 224(1):58-69.
Mougiakakos D, Kronke G, Volkl S et al. CD19-Targeted CAR T Cells in Refractory Systemic Lupus Erythematosus. N Engl J Med. 2021; 5: 385(6):567-569
Musolino A, Naldi N, Bortesi B. et al. Immunoglobulin G fragment C receptor polymorphisms and clinical efficacy of trastuzumab-based therapy in patients with HER-2/neu-positive metastatic breast cancer. J Clin Oncol. 2008; 26(11):1789-1796. doi:10.1200/JCO.2007.14.8957
Nishimoto N, Terao K. Mima T. et al: Mechanisms and pathologic significances in increase in serum interleukin-6 (IL-6) and soluble IL-6 receptor after administration of an anti-IL-6 receptor antibody, tocilizumab, in patients with rheumatoid arthritis and Castleman disease. Blood 2008; 1112:3959-3964.
Oh S and Payne A S. Engineering Cell Therapies for Autoimmune Diseases: From Preclinical to Clinical Proof of Concept. Immune Netw. 2022: 22(5): e37: doi: 10.4110/1n.2022.22.e37; pISSN 1598-2629-eISSN 2092-6685
Parra Sinchez, A. R., Voskuyl, A. E. & van Vollenhoven. R. F. Treat-to-target in systemic lupus erythematosus: advancing towards its implementation. Nat. Rev. Rheumatol. 18, 146-157 (2022)
Petri M, Kim M Y, Kalunian K C, et al. Combined oral contraceptives in women with systemic lupus erythematosus. N Engl J Med 2005; 353:2550-8
Pisetskv D S. Immune response to DNA in systemic lupus erythematosus. Isr Med Assoc J. 2001; (3)(11): 850-853
Pons-Estel G J, Serrano R, Plasin M A et al. Epidemiology and management of refractory lupus nephritis. Autoimmun Rev. 2011: 10(11): 655-663
Reagan P M, Friedverg J W. Axicabtagene ciloleucel and brexucabtagene autoleucel in relapsed and refractory diffuse large B-cell and mantel cell lymphomas. Future Oncol. 2021; 17(11):1269-1283.
Reddy, V.; Klein, C.; Isenberg, D. A.; Glennie, M. J.; Cambridge, G.; Cragg, M. S.; Leandro, M. J. Obinutuzumab induces superior B-cell cytotoxicity to rituximab in rheumatoid arthritis and systemic lupus erythematosus patient samples. Rheumatology 2017, 56, 1227-1237.
Rees F, Doherty M, Grainge M J et al. The worldwide incidence and prevalence of systemic lupus erythematosus: a systematic review of epidemiological studies Rheumatology. Oxford 2017: 56: 1945-1961
RITUXAN—rituximab injection, solution prescribing information, revised June 2023
Romagnani P, Remuzzi G, Glassock R et al. Chronic kidney disease. Nat Rev Dis Primers. 2017; 3, 17088
Rosman Z, Shoenfeld Y, Zandman-Goddard G. Biologic therapy for autoimmune diseases: an update. BMC Med 2013; 11:88.
Rovin B H, Furie R, Latinis K, et al. Efficacy and safety of rituximab in patients with active proliferative lupus nephritis: the Lupus nephritis assessment with rituximab study. Arthritis Rheum. 2012; 64(4):1215-1226. doi: 10.1002/art.34359
Rubnitz J E, Inaba H, Ribeiro R C, et al. NKAML: a pilot study to determine the safety and feasibility of haploidentical natural killer cell transplantation in childhood acute myeloid leukemia. J Clin Oncol. 2010:28(6):955-959. doi:10.1200/JCO.2009.24.4590
Singh S and Saxena R. Lupus nephritis. Am J Med Sci. 2009; 337(6): 451=460
Stach C M, Sloan V S, Woodworth T G, Kilgallen B, Furst D E. Rheumatology Common Toxicity Criteria (RCTC): An Update Reflecting Real-World Use. Drug Saf 2019 December:42(12):1499-1506. doi: 10.1007/s40264-019-00864-9. PMID: 31696432.
Stojan G and Petri M Epidemiology of systemic lupus erythematosus: an update. Curr Opin Rheumatol. 2018; 30(2): 144-150
Terme M, Ullrich E, Delahaye N F, Chaput N, Zitvogel L. Natural killer cell-directed therapies: moving from unexpected results to successful stratagies. Nat Immunol. 2008; 9(5):486-494.
Touma Z, Urowitz M B, Gladman D D. SLEDAI-2K for a 30-day window. Lupus 2010; 19:49-51
Veluchamy J P, Kok N, van der Vliet H J, Verheul H M W, de Gruijl T D, Spanholtz J. The Rise of Allogeneic Natural Killer Cells As a Platform for Cancer Immunotherapy: Recent Innovations and Future Developments. Front Immunol. 2017; 8:631. Published 2017 May 31. doi:10.3389/fimmu.2017.00631
Vivier E, Tomasello E. Baratin M, Walzer T, Ugolini S. Functions of natural killer cells. Nat Immunol. 2008 May; 9(5):503-10. doi: 10.1038/ni1582. PMID: 18425107.Weidenbusch M, Rommele C, Schrottle A, Anders H J. Beyond the LUNAR trial. Efficacy of rituximab in refractory lupus nephritis. Nephrol Dial Transplant. 2013; 28(1):106-111
Weidenbusch M. Rommele C, Schrottle A, Anders H J. Beyond the LUNAR trial. Efficacy of rituximab in refractory lupus nephritis. Nephrol Dial Transplant. 2013; 28(1):106-111. doi:10.1093/ndt/gfs285
Xie, G., Dong, H., Liang, Y., Ham, J. D., Rizwan, R., & Chen, J. (2020). CAR-NK cells: A promising cellular immunotherapy for cancer. EBioMedicine, 59, 102975.
Yo J H, Barbour T D and Nicholls K. Management of refractory lupus nephritis challenges and solutions. Rheumatol. 2019; 12; 11:179-188
Zhang W, Feng J, Cinquina A, Wang Q, Xu H, Zhang Q, Sun L, Chen Q. Xu L, Pinz K, et al. Treatment of systemic lupus erythematosus using BCMA-CD19 compound CAR. Stem Cell Rev Rep 2021; 17:2120-2123.
Zhao Y, Niu C. Cui J. Gamma-delta (γδ) T cells: friend or foe in cancer development?J Transl Med. 2018; 16(1):3.
Myasoedova E. Davis J, Matteson E L, Crowson C S. Is the epidemiology of rheumatoid arthritis changing?Results from a population-based incidence study, 1985-2014. Ann Rheum Dis. 2020 April; 79(4):440-444. doi: 10.1 136/annrheumdis-2019-216694. Epub 2020 Feb. 17. PMID: 32066556; PMCID: PMC7085464.
Melville A R, Kearsley-Fleet L, Buch M H, Hyrich K L. Understanding Refractory Rheumatoid Arthritis: Implications for a Therapeutic Approach. Drugs. 2020 June; 80(9):849-857. doi: 10.1007/s40265-020-01309-9. PMID: 32361822.
Abulikemu K, Hu F. Liang J, Kang X. Targeting therapy in pemphigus: Where are we now and where are we going?Heliyon. 2023 May 25; 9(6):e16679. doi: 10.1016/j.heliyon.2023.e16679. PMID: 37292301; PMCID: PMC10245244.
Koike H, Nishi R, Ohyama K. Morozumi S, Kawagashira Y, Furukawa S, Mouri N, Fukami Y, Iijima M, Sobue G. Katsuno M. ANCA-Associated Vasculitic Neuropathies: A Review. Neurol Ther. 2022 March; 11(1):21-38. doi: 10.1007/s40120-021-00315-7. Epub 2022 Jan. 19. PMID: 35044596; PMCID: PMC8857368.
Jonathan Kay, Katherine S. Upchurch, ACR/EULAR 2010 rheumatoid arthritis classification criteria, Rheumatology, Volume 51, Issue suppl_6, December 2012, Pages vi5-vi9, doi.org/10.1093/rheumatology/kes279
Shimizu. T., Takebayashi. T., Sato, Y., Niizeki. H., Aoyama. Y., Kitajima. Y., Iwatsuki, K., Hashimoto, T., Yamagami, J., Werth, V. P., Amagai, M. and Tanikawa, A. (2014), Grading criteria for disease severity by pemphigus disease area index. J Dermatol, 41: 969-973. doi.org/10.1111/1346-8138.12649

### VIII. EXAMPLES

The following examples are included for illustrative purposes only and are not intended to limit the scope of the invention.

### Example 1: AB-101

AB-101 is a universal, off-the-shelf, cryopreserved allogeneic cord blood derived NK cell therapy product comprising ex vivo expanded and activated effector cells designed to enhance ADCC anti-tumor responses in patients, e.g., patients treated with monoclonal antibodies or NK cell engagers. AB-101 was prepared as described, for example, in WO2022/133056.

### Example 2: AB-101 in Combination with Anti-CD20 Antibody Mediates Killing of Healthy Donor B Cells In Vitro

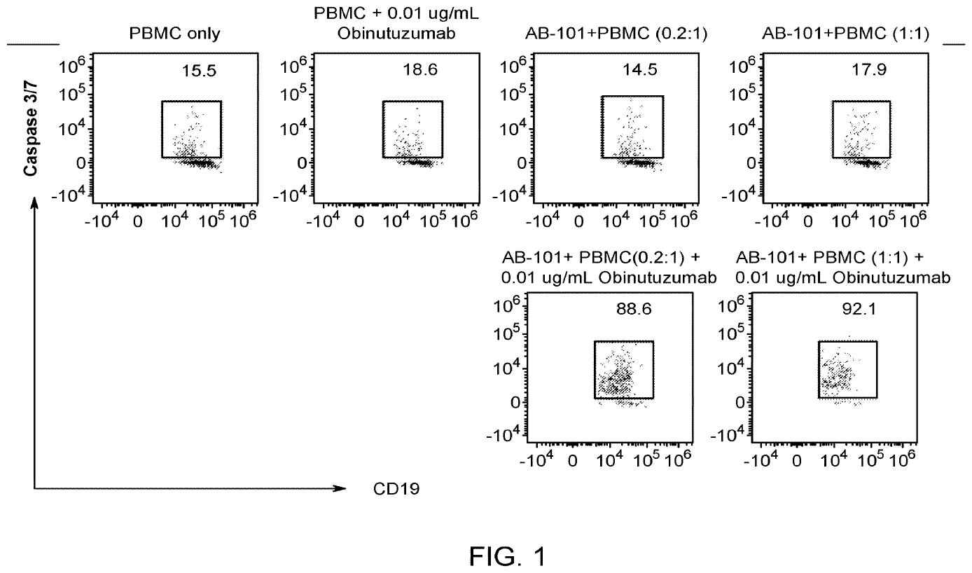

AB-101 was combined with healthy donor PBMC (0.2:1 or 1:1 ratio) and combined with or without antibody in a 4-hour killing assay. Donor B cells (CD19+CD20+) were detected by flow cytometry and the percentage of apoptotic cells was detected with caspase 3/7 dye. Number in the plot shows the percentage of caspase-positive B cells. Results show that AB-101 in combination with anti-CD20 antibody (bottom panel) results in ADCC and increased B cell death versus Obinutuzumab alone or AB-101 plus PBMC (no antibody) in the top panel. As shown in FIG. 1, the combination mediated killing of healthy donor B cells.

### Example 3: AB-101 in Combination with Anti-CD19 or Anti-CD20 Antibody Mediates Killing of Healthy Donor B Cells In Vitro

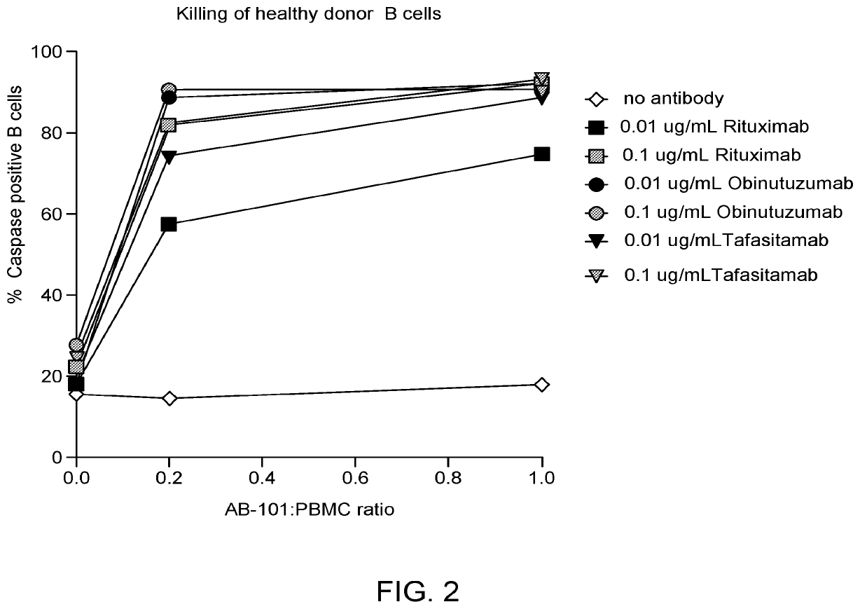

AB-101 was combined with healthy donor PBMC (0.2:1 or 1:1 ratio) and combined with or without antibody at 0.01 or 0.1 ug/mL in a 4-hour killing assay. Donor B cells (CD19+CD20+) were detected by flow cytometry and the percentage of apoptotic cells was detected with caspase 3/7 dye. Results show that AB-101 in combination with antibody results in ADCC and increased B cell death versus PBMC+antibody in the absence of AB-101 (symbols on the y-axis). Obinutuzumab in combination with AB-101 was the most potent combination to demonstrate B cell killing. As shown in FIG. 2, AB-101 in combination with anti-CD19 (Tafasitamab) or anti-CD20 antibody (Rituximab or Obinutuzumab) mediates killing of healthy donor B cells in vitro.

ADCC of AB-101 was evaluated against healthy human peripheral blood mononuclear cells (PBMCs) and PBMCs isolated from SLE patients, in combination with anti-CD20 (rituximab and obinutuzumab) or anti-CD19 (tafasitamab) monoclonal antibodies. PBMCs were incubated with or without antibodies at different effector (NK cell) to target (PBMCs) ratios. Apoptotic (caspase positive) cells were quantified by flow cytometry.

Whole blood from healthy donors or SLE donors was collected in ACD-A anticoagulant and shipped overnight at ambient temperature. PBMCs were isolated and viable cell number was determined. PBMCs were resuspended in assay medium and plated at 2×10⁵ cells/well. AB-101 was thawed in a water bath at 37° C. Once thawed, AB-101 was washed in culture medium, centrifuged, and a cell aliquot was counted. AB-101 cell number was adjusted to 5×10⁶ cells/mL. AB-101 was further diluted in culture medium and added to the PBMCs at 4×10⁴ cells/well or 2×10⁵ cells/well, yielding E:T ratios of 1:1 and 0.2:1. Antibodies (0.01, 0.1, or 1 μg/mL of rituximab, obinutuzumab, or tafasitamab) were added to the co-culture and the plates were incubated for 3.5 hours in a 37° C. incubator with 5% CO₂. Caspase 3/7 green was added to each well and incubated for an additional 30 minutes at 37° C. Following the incubation period, the co-culture was prepared for analysis by flow cytometry. B cells were identified by as CD19+ cells for the rituximab and obinutuzumab treated cells and CD20+ for the tafasitamab treated cells due to antibody interference with the flow cytometry detection antibodies. The specific lysis (%) of target cells was calculated as follows: Specific lysis (%)=(Sample well-spontaneous)/(100-spontaneous)*100.

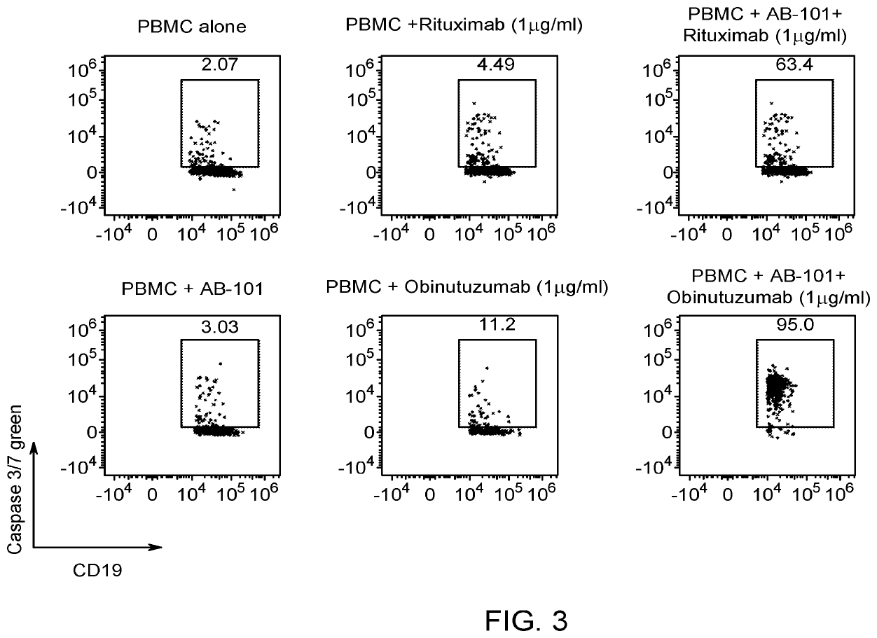

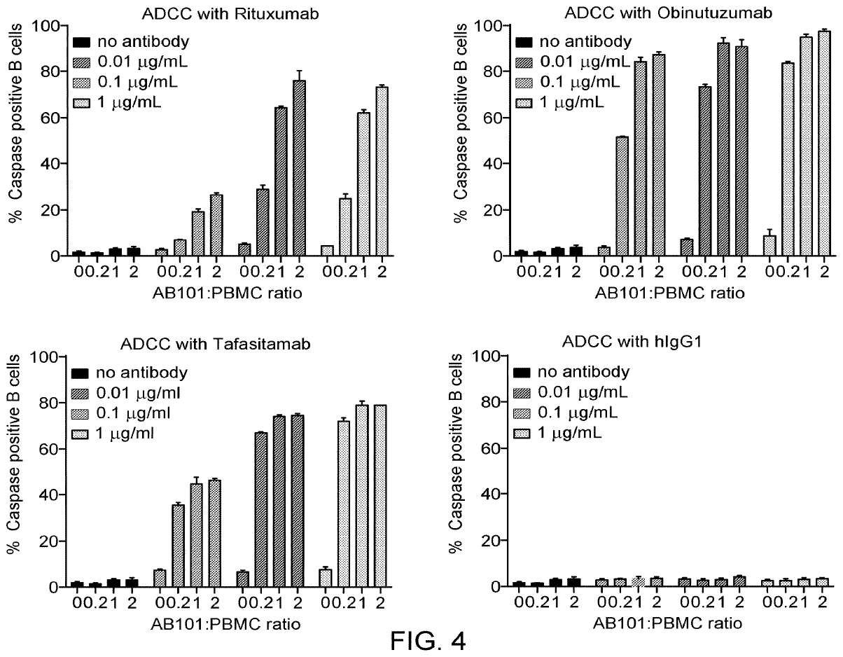

Cytotoxicity experiments utilizing healthy human PBMCs co-cultured with AB-101 demonstrated enhanced AB-101-mediated B cell killing at different E:T ratios in the presence of anti-CD20 or anti-CD19 antibodies (Table 6). Obinutuzumab, in combination with AB-101, was more effective at inducing B cell apoptosis at lower E:T ratios than rituximab or tafasitamab, presumably through enhanced antibody glycoengineering. Little to no increase in B-cell apoptosis was noted with PBMCs and AB-101 alone (Table 6). PBMCs isolated from SLE patient samples (n=3) were tested in the same co-culture system with AB-101 as the normal human PBMCs. Anti-CD20 or anti-CD 19 antibodies were added to the co-culture and B cell apoptosis was measured. Enhanced ADCC against SLE B cells was observed when AB-101 was combined with anti-CD20 or anti-CD19 antibodies in an antibody concentration-dependent and an E:T ratio-dependent manner (FIG. 3 and FIG. 4). At a 1:1 E:T ratio with 1 μg/mL of antibody, enhanced cytotoxicity of SLE B cells was observed with obinutuzumab (range 78.6-95.4%), tafasitamab (range 24.2-76.92), and Rituximab (19.2-62.23) versus minimal B cell killing with human IgG1 control antibody (range 2.3-8.3% o) (Table 7, representative donor). The specificity of cell killing in the SLE PBMC sample was evaluated by examining the effects of the combination on SLE T-cells. Little to no off-target apoptosis was observed in this cell population in the presence of AB-101 and any of the three antibodies tested (Table 7, representative donor).

In conclusion, AB-101, in combination with anti-CD 19 and anti-CD20 mAbs, was shown to kill SLE B-cells via an ADCC mechanism. The killing was specific to B-cells, as no significant effect on SLE T-cells was observed under the same co-culture conditions.

TABLE 6
Effect of AB-101 and anti-CD19 or anti-CD20
treatment on the percentage of healthy donor
Human B- and T-cells (Summary of all donors)

| Healthy Donors | Percent Caspase Positive B cells | | | | | |
|---|---|---|---|---|---|---|
| Condition | PBMC alone | | E:T (0.2:1) | | E:T (1:1) | |
| (μg/ml of Ab) | Avg | SD | Avg | SD | Avg | SD |
| No Ab (0) | 3.82 | 1.92 | 3.77 | 1.84 | 4.11 | 1.34 |
| Ritux (0.01) | 3.32 | 2.44 | 5.81 | 2.91 | 12.87 | 3.80 |
| Ritux (0.1) | 3.67 | 2.60 | 11.98 | 5.21 | 32.20 | 12.07 |
| Ritux (1) | 3.90 | 2.51 | 13.77 | 4.73 | 38.13 | 9.47 |
| Obin (0.01) | 3.91 | 1.47 | 29.20 | 13.97 | 61.50 | 1.88 |
| Obin (0.1) | 5.60 | 2.48 | 34.30 | 13.78 | 71.43 | 3.60 |
| Obin (1) | 7.73 | 3.33 | 51.70 | 6.89 | 79.10 | 3.23 |
| Tafa (0.01) | 4.81 | 2.62 | 25.98 | 11.63 | 57.83 | 10.07 |
| Tafa (0.1) | 4.35 | 2.34 | 28.20 | 8.55 | 63.55 | 7.28 |
| Tafa (1) | 4.92 | 2.07 | 26.75 | 9.15 | 61.55 | 6.40 |
| hIgG1 (0.01) | 3.35 | 1.01 | 3.95 | 1.52 | 5.32 | 2.05 |
| hIgG1 (0.1) | 3.85 | 1.41 | 4.20 | 1.32 | 5.46 | 1.46 |
| hIgG1 (1) | 3.98 | 1.40 | 4.49 | 2.05 | 5.85 | 1.51 |
| | Percent Caspase Positive T cells | | | | | |
| Condition | PBMC alone | | E:T (0.2:1) | | E:T (1:1) | |
| (μg/ml of Ab) | Avg | SD | Avg | SD | Avg | SD |
| No Ab (0) | 3.09 | 2.77 | 3.68 | 2.83 | 6.36 | 3.21 |
| Ritux (0.01) | 3.04 | 2.73 | 3.66 | 2.90 | 6.28 | 3.17 |
| Ritux (0.1) | 3.33 | 2.98 | 3.64 | 2.91 | 6.11 | 2.91 |
| Ritux (1) | 3.16 | 2.69 | 4.02 | 3.09 | 6.07 | 3.19 |
| Obin (0.01) | 3.28 | 2.72 | 4.07 | 3.24 | 6.19 | 3.09 |
| Obin (0.1) | 3.41 | 2.83 | 4.35 | 3.42 | 6.95 | 3.32 |
| Obin (1) | 3.81 | 2.76 | 4.97 | 3.33 | 8.07 | 4.09 |
| Tafa (0.01) | 2.81 | 2.45 | 3.69 | 3.09 | 6.30 | 3.25 |
| Tafa (0.1) | 2.99 | 2.66 | 3.55 | 2.76 | 6.25 | 3.38 |
| Tafa (1) | 2.94 | 2.51 | 3.84 | 3.12 | 6.32 | 3.31 |
| hIgG1 (0.01) | 3.17 | 2.64 | 3.95 | 3.29 | 6.67 | 3.54 |
| hIgG1 (0.1) | 3.71 | 3.05 | 3.97 | 3.36 | 6.89 | 3.40 |
| hIgGl (1) | 4.04 | 3.25 | 4.59 | 3.31 | 7.40 | 4.00 |

Ritux = rituximab,
Obin = obinutuzumab,
Tafa = tafasitamab,
E:T = AB-101:PBMC,
Avg = average,
SD = standard deviation

FIG. 3 shows representative FACS plots showing the gating of dead/dying caspase 3/7⁺ CD19⁺SLE B cells in PBMC alone (upper left) or PBMC plus AB-101 (bottom left), with anti-CD20 antibodies (center) or with AB-101 plus anti-CD20 antibodies at a 1:1 E:T ratio (right). Lymphocytes were first gated by forward scatter (FSC), side scatter (SSC), followed by single cells. B cells were gated as CD45⁺CD14⁻CD3⁻CD56⁻CD16⁻ and CD19⁺.

SLE patient PBMCs were isolated from peripheral blood and combined with thawed AB-101, with or without the anti-CD20, rituximab or obinutuzumab (top panel), or anti-CD19, tafasitamab, or human IgG1 isotype control (bottom panel), for 4 hours. The percentage of caspase positive B cells was determined by flow cytometry. FIG. 4. Data are expressed as the mean±SD of duplicate wells.

TABLE 7
Effect of AB-101 and anti-CD19 or anti-CD20 treatment
on the percentage of SLE B- and T-cells

| SLE Donor | Percent Caspase Positive B cells | | | | | | | |
|---|---|---|---|---|---|---|---|---|
| Condition | PBMC alone | | E:T (0.2:1) | | E:T (1:1) | | E:T (2:1) | |
| (μg/ml of Ab) | Avg | SD | Avg | SD | Avg | SD | Avg | SD |
| No Ab (0) | 1.84 | 0.33 | 1.4 | 0.08 | 3.14 | 0.15 | 3.34 | 0.88 |
| Ritux (0.01) | 2.77 | 0.89 | 6.94 | 0.17 | 19.40 | 1.13 | 26.70 | 0.57 |
| Ritux (0.1) | 5.10 | 0.80 | 29.2 | 1.98 | 64.70 | 0.14 | 76.15 | 5.87 |
| Ritux (1) | 4.49 | 0.01 | 25 | 2.55 | 62.20 | 1.70 | 73.75 | 0.35 |
| Obin (0.01) | 3.63 | 0.40 | 51.75 | 0.07 | 84.50 | 2.12 | 87.65 | 1.20 |
| Obin (0.1) | 7.09 | 0.54 | 74 | 0.57 | 92.40 | 3.25 | 91.10 | 3.68 |
| Obin (1) | 8.60 | 3.68 | 84 | 0.42 | 95.40 | 0.57 | 97.65 | 1.06 |
| Tafa (0.01) | 7.59 | 0.34 | 36.9 | 1.27 | 69.55 | 0.21 | 74.75 | 1.20 |
| Tafa (0.1) | 6.63 | 0.92 | 46.45 | 3.75 | 76.40 | 0.71 | 81.60 | 1.84 |
| Tafa (1) | 7.73 | 1.60 | 48.1 | 0.14 | 76.85 | 0.49 | 81.90 | 0.00 |
| hIgG1 (0.01) | 3.09 | 0.06 | 3.37 | 0.20 | 3.67 | 0.88 | 3.90 | 0.35 |
| hIgG1 (0.1) | 3.53 | 0.16 | 3.02 | 0.25 | 3.38 | 0.51 | 4.45 | 0.16 |
| hIgG1 (1) | 2.99 | 0.47 | 2.93 | 0.85 | 3.25 | 0.68 | 3.62 | 0.29 |
| | Percent Caspase Positive T cells | | | | | | | |
| Condition | PBMC alone | | E:T (0.2:1) | | E:T (1:1) | | E:T (2:1) | |
| (μg/ml of Ab) | Avg | SD | Avg | SD | Avg | SD | Avg | SD |
| No Ab (0) | 0.70 | 0.18 | 0.68 | 0.04 | 2.01 | 0.21 | 2.87 | 0.31 |
| Ritux (0.01) | 1.11 | 0.10 | 1.11 | 0.10 | 2.08 | 0.17 | 2.77 | 0.29 |
| Ritux (0.1) | 1.01 | 0.00 | 1.31 | 0.03 | 2.29 | 0.18 | 3.25 | 0.06 |
| Ritux (1) | 1.16 | 0.10 | 1.83 | 0.25 | 3.58 | 0.24 | 4.85 | 0.23 |
| Obin (0.01) | 1.25 | 0.13 | 1.44 | 0.01 | 2.48 | 0.01 | 3.55 | 0.04 |
| Obin (0.1) | 1.52 | 0.13 | 1.95 | 0.01 | 6.30 | 0.18 | 9.86 | 0.49 |
| Obin (1) | 1.52 | 0.04 | 3.08 | 0.28 | 10.10 | 0.43 | 14.90 | 0.57 |
| Tafa (0.01) | 1.92 | 0.20 | 2.17 | 0.19 | 3.83 | 0.63 | 5.42 | 0.06 |
| Tafa (0.1) | 1.77 | 0.12 | 2.06 | 0.09 | 3.68 | 0.22 | 5.66 | 0.14 |
| Tafa (1) | 1.33 | 0.11 | 1.71 | 0.11 | 2.60 | 0.18 | 4.29 | 0.24 |
| hIgG1 (0.01) | 1.44 | 0.17 | 1.61 | 0.03 | 2.52 | 0.02 | 3.91 | 0.04 |
| hIgG1 (0.1) | 1.58 | 0.13 | 1.75 | 0.14 | 2.73 | 0.25 | 4.09 | 0.04 |
| hIgG1 (1) | 1.48 | 0.23 | 1.86 | 0.04 | 2.77 | 0.08 | 3.87 | 0.64 |

Ritux = rituximab,
Obin = obinutuzumab,
Tafa = tafasitamab,
hIgG1 = control Human Immunoglobulin G1,
E:T = AB-101:PBMC,
Avg = average,
SD = standard deviation

### Example 4: AB-101 in Combination with Anti-CD20 Antibody Results in Minimal Killing of T Cells

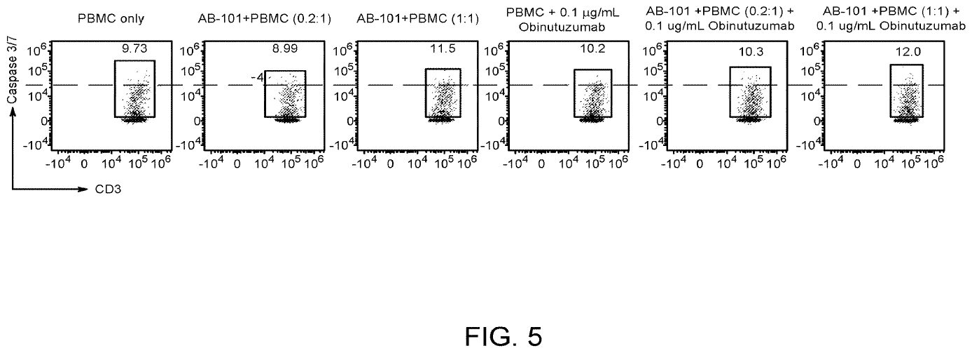

AB-101 was cultured with healthy donor PBMC and with or without antibody. After 4 hours, the percentage of caspase positive T cells (CD3+) was determined by flow cytometry. Addition of AB-101 and Obinutuzumab results in minimal increase in T cell killing versus PBMC alone. As shown in FIG. 5, AB-101 in combination with anti-CD20 antibody (Obinutuzumab) results in minimal killing of T cells (CD3±).

### Example 5: AB-101 in Combination with Anti-CD20 Antibody Mediates Killing of SLE Donor B Cells in a 4-Hour Cytotoxicity Assay

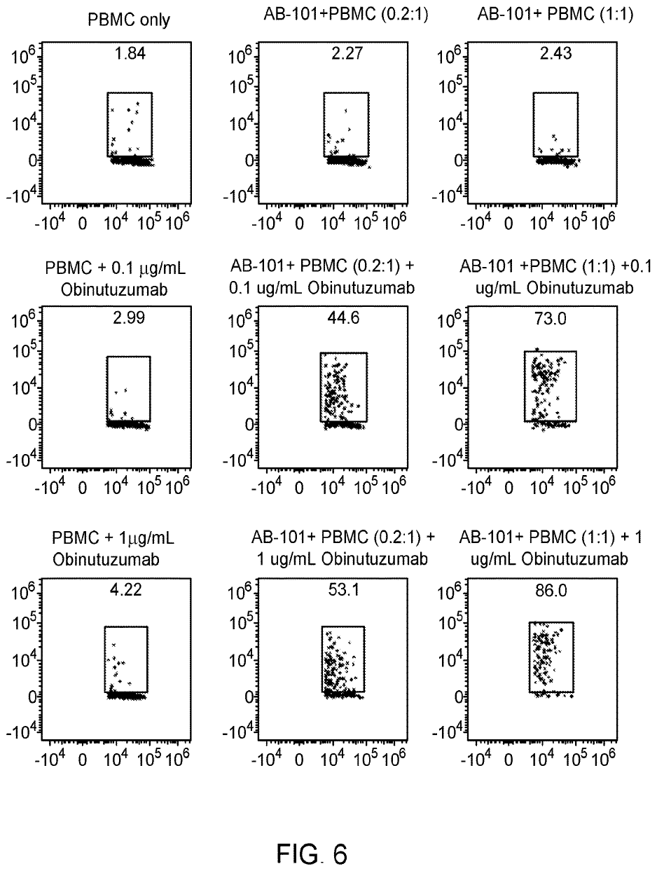

AB-101 was cultured with SLE donor PBMC and with or without antibody. After 4 hours, the percentage of caspase positive B cells (CD19+) was determined by flow cytometry. Combination of AB-101 plus Obinutuzumab (0.1 or 1 μg/mL) results in increased killing of SLE donor B cells. The higher ratio of AB-101 results in more B cell killing in the presence of antibody. As shown in FIG. 6, AB-101 in combination with anti-CD20 antibody (Obinutuzumab) mediates killing of SLE donor B cells in a 4-hour cytotoxicity assay.

### Example 6: Humanized NSG Mouse Model

In order to evaluate ADCC with the combination of AB-101 and the anti-CD20 mAb, Obinutuzumab, against human B-cells in vivo, a CD34+ humanized NSG (huNSG) mouse model was used. The CD34+ huNSG mouse model is specialized to study human hematopoiesis and the immune system. In brief, this model consists of NSG mice being conditioned with whole body irradiation followed by engraftment with human cord blood-derived CD34+ hematopoietic stem cells (Ishikawa 2005). These huNSG mice have high human B-cell levels, and moderate T-cell levels, as well as a small population of myeloid cells but few NK cells in the peripheral blood at ~12-16 weeks post-engraftment. Given the high level of human B-cells, this model was considered relevant for assessing treatment effects on B-cell levels and general animal health.

19-23 week-old female CD34+ Humanized NSG (NOD.Cg-Prkd^{scid} Il2rg^{tn1Wjl}/SzJ) mice were used as experimental recipients. Upon receipt, animals were allowed a 7-day acclimation period prior to being placed on study. Animals were assigned to a treatment group on Day 0 based on human CD45+CD19+B-cell engraftment levels to ensure the group mean was similar across groups.

The selected dose and schedule for administration of AB-101 and Obinutuzumab was based on previously published preclinical studies using Obinutuzumab in a similar humanized model (Bacac M, 2018), as well as internal studies with B-cell lymphoma xenografts and pilot studies with huNSG mice. The selected 150 μg/kg Obinutuzumab dose was considered a dose level that partially depletes human B-cells, leaving a window to observe ADCC with the combination.

The dosing schema is shown in FIG. 10. In the single agent groups, AB-101 (1×10⁷ cells/dose) was administered as a single slow bolus intravenous injection (IV) to huNSG mice on Day 0 and 7 and obinutuzumab was given by intraperitoneal (IP) injection on Day 0 or Day 0 and 7. For the combination groups, obinutuzumab was administered first, followed by AB-101, on Day 0 or Day 0 and 7. Vehicle animals were treated like the combination group, receiving IP and IV injections on Day 0 and 7. Peripheral blood was collected on day −2 (baseline), Day 7, 14, 21 and 28. The Day 7 blood collection was taken prior to the second administration of any of the test articles. Depletion of human B-cells was assessed in peripheral blood and tissues following treatment with AB-101, obinutuzumab or the combination of AB-10l and obinutuzumab. Toxicity was assessed through mortality/cage side observations, clinical observations, body weight measurements, clinical chemistry (alanine aminotransferase, aspartate aminotransferase, alkaline aphosphatase, blood urea nitrogen, creatinine, calcium, total bilirubin, phosphorus, total protein, lipemia index, hemolysis index), macroscopic and microscopic evaluation of organs and select tissues.

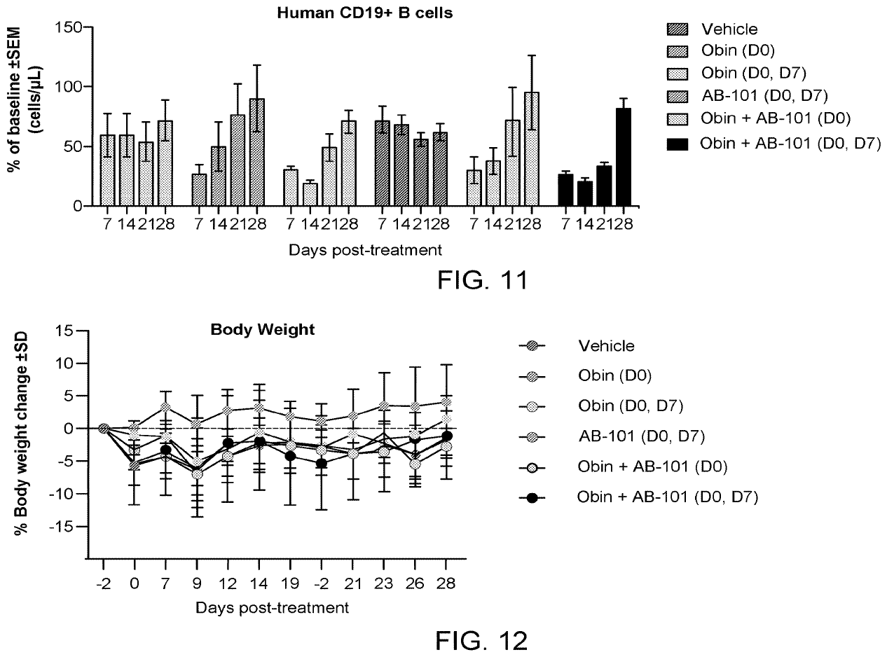

Efficacy of AB-101, obinutuzumab and AB-101 in combination with obinutuzumab was assessed by weekly monitoring of the change in CD19+B-cell levels in peripheral blood from baseline (Day −2) following treatment (FIG. 11). On Day 7, the combination of a single dose of obinutuzumab+AB-101 led to a comparable depletion of CD19+B cells (average 28.15%+4, Groups 5 and 6, n=8) vs obinutuzumab alone (average 29.65%±3, Groups 2 and 3, n=8). However, when comparing the percent change in CD19+B-cells on Day 21 for two doses of either obinutuzumab (average 49.3%±11, n=5) or the combination of AB-101 and obinutuzumab (average 33.6%±3, n=5), there was a trend towards enhanced B-cell depletion with the combination (Table 8). For comparison, the percent change in CD19+B-cells from baseline in vehicle and AB-101 alone group had an average of 59.67±17.6 and 72.3%±11.1, respectively. Little to no off-target apoptosis (T cells, myeloid cells) was observed in the presence of AB-101 alone or in combination with obinutuzumab.

TABLE 8
Effect of AB-101 and Obinutuzumab on the percent
change of CD19+ B cells from baseline (Day-2)

| % baseline, CD19+ | | | | | | | | | | |
|---|---|---|---|---|---|---|---|---|---|---|
| (cells/μl) | | | Study Day | | | | | | | |
| | Group | | D7 | | D14 | | D21 | | D28 | |
| Group | Name | N | AVG | SEM | AVG | SEM | AVG | SEM | AVG | SEM |
| 1 | Vehicle(s) | 3 | 59.5 | 17.7 | 59.5 | 16.7 | 54.0 | 16.2 | 71.6 | 16.8 |
| 2 | Obin, D0 | 4 | 27.3 | 7.7 | 50.1 | 20.2 | 76.3 | 25.9 | 89.7 | 27.7 |
| 3 | Obin, D0, 7 | 5 | 31.1 | 2.4 | 18.9 | 2.6 | 49.3 | 11.0 | 70.5 | 9.7 |
| 4 | AB-101 | 4 | 72.3 | 11.1 | 68.3 | 7.3 | 56.7 | 4.6 | 61.7 | 7.9 |
| 5 | Obin + AB- | 3 | 30.9 | 10.7 | 38.1 | 10.9 | 70.6 | 28.5 | 95.5 | 31.0 |
| | 101, D0 | | | | | | | | | |
| 6 | Obin, AB- | 5 | 26.5 | 2.8 | 21.4 | 2.4 | 33.6 | 3.0 | 81.8 | 8.3 |
| | 101, D0, 7 | | | | | | | | | |

Data on Day 7 is following a single dose of Obin, AB-101 or obin+AB-101 and Day 14 is following two doses of obin, AB-101 or obin+AB-101 for the indicated groups. In the text Day 7 data for Groups 2 and 3 and Groups 5 and 6 are averaged since the test articles were administered once at this timepoint. Comparable depletion of CD19+B-cells was observed with obinutuzumab alone (average 29.65%±3, n=8) vs obinutuzumab+AB-101 (average 28.15%±4, n=8). Obin=obinutuzumab, AVG=average, SEM=standard error of the mean.

The effect of treatment on toxicity endpoints was evaluated. No mortality or clinical observations were noted in any of the treatment groups. There was no significant difference in percent body weight change between vehicle, obinutuzumab, AB-101, or the combination of AB-101 and obinutuzumab treatment groups (FIG. 12). There were no AB-101- or obinutuzumab-related effects, as single agents or in combination, among clinical chemistry parameters across all groups. Fluctuations among individual and mean values were considered sporadic, consistent with biologic variation and/or negligible in magnitude, and not related to test article administration.

At necropsy, there were no test article-related macroscopic findings in the organs from any of the treatment groups. No microscopic findings related to AB-101 or obinutuzumab administration were reported.

In summary, the efficacy and tolerability of AB-101, in combination with the anti-CD20 mAb, obinutuzumab, was assessed in a CD34+ humanized NSG mouse model. Given the high level of human B-cells, it was considered relevant for assessing treatment effects on B-cell levels and general animal health. AB-101, in combination with obinutuzumab, demonstrated a trend towards enhanced B-cell depletion compared to AB-101 or obinutuzumab alone. Little to no off-target apoptosis (T-cells, myeloid cells) was observed in vivo and no toxicity associated with the combination was noted.

The NSG mouse model was used to determine the biodistribution and pharmacokinetics (PK) of AB-101. Vehicle (PBS. Dextran. Albumin (human). DMSO) and AB-101 cells (0.5×10⁷ cells/mouse, 2×10⁷ cells/mouse) were administered intravenously (0.25 mL/mouse) for a total of 8 doses. Animals in vehicle and AB-101 groups were sacrificed at timepoints 4 hr, 1, 3, 7, 14 and 78 days (n=3 male mice, n=3 female mice per timepoint) post last dose infusion.

Pharmacokinetics of AB-101 was determined using a qPCR method. A set of primers/probe specific to human β-globin gene were used to detect AB-101 DNA in the background of NSG mouse matrix DNA.

AB-101 cells were detected predominantly in highly perfused tissues (lungs, spleen, heart and liver) and at the site of injection starting at 4 hrs after administration, until 3 days after administration of final dose of AB-101 (day 53). 7 days after administration of final dose (day 57) AB-101 cells were detected in lung (3 out of 6 samples), spleen (5 out of 6 samples) and injection site (5 out of 6 samples). At 14 days and 28 days after administration of final dose (day 64 and day 78 respectively), AB-101 cells were detected in two and one injection site samples, respectively. The sporadic incidence and low concentrations observed from the injection site samples at day 64 and day 78 would not be indicative of systemic persistence of AB-101.

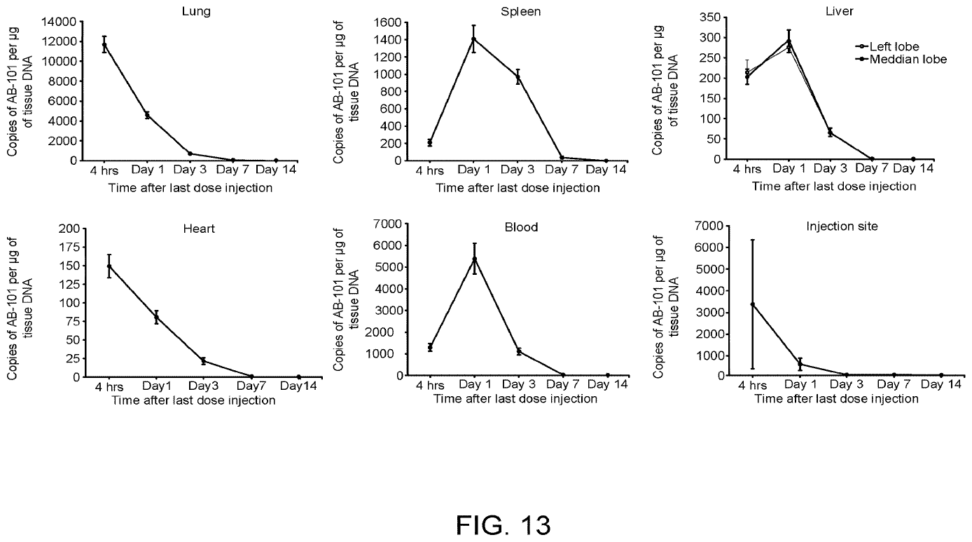

The results from the biodistribution studies indicate that the distribution of AB-101 cells in vivo is consistent with the intravenous route of administration of cellular products and that the cells lack long-term persistence potential and were cleared after 7 days post-administration with no evidence of permanent engraftment. The pharmacokinetic profile of AB-101 is depicted in FIG. 13.

Toxicology studies were performed as a dose range finding study (DRF) and a GLP-toxicology study. The objective of the DRF study was to identify the safe dose range of AB-101 cells in NSG mice after multiple intravenous doses. The objective of the GLP toxicity study was to evaluate the toxicity of AB-101 in NSG mice after repeated intravenous administration of doses identified in DRF study.

The mice carry two mutations on the NOD/ShiLtJ genetic background: severe combined immune deficiency (scid) and a complete null allele of the IL2 receptor common gamma chain (IL2rgnull). The scid mutation is in the DNA repair complex protein Prkdc and renders the mice B and T cell deficient. The IL2rgnull mutation prevents cytokine signaling through multiple receptors, leading to a deficiency in functional NK cells.

The objective of the DRF study was to identify the safe dose range of AB-101 cells in NSG mice after multiple intravenous doses. The regimen tested was 8 weekly intravenous administrations via tail vein. The proposed test dose range in the preclinical toxicology assessments was calculated to deliver a greater exposure of the AB-101 product than the equivalent human dose of 4×10⁹ cells per dose. The dose range of 0.1×10⁷ to 2.5×10⁷ cells/dose/animal tested in the DRF study provides sufficient safety margin with expected clinical dosing. Mouse to human doses were scaled allometrically (Nair 2016): the mid dose level of 0.5×10⁷ cells/animal corresponds to 14×10⁹ cells and high dose level of 2.5×10⁷ cells/animal corresponds to 70×10⁹ cells of human equivalent dosing for a patient of 70 kg. The outcome of the DRF study was used to inform the GLP toxicology study dosing. The experimental design for the DRF study is shown in Table 9.

TABLE 9
Experimental Design

| | | | Dose | Dose | | |
|---|---|---|---|---|---|---|
| Group | | Dose Level | Volume | Concentration | No. of | No. of |
| No. | Treatment | (cells/animal) | (mL) | (cells/mL) | Males | Females |
| 1 | Vehicle Control | NA | 0.25 | 0 | 6 | 6 |
| 2 | AB-101 Dose 1 | 0.1 × 10⁷ | 0.25 | 0.4 × 10⁷ | 6 | 6 |
| 3 | AB-101 Dose 2 | 0.5 × 10⁷ | 0.25 | 2 × 10⁷ | 6 | 6 |
| 4 | AB-101 Dose 3 | 2.5 × 10⁷ | 0.25 | 10 × 10⁷ | 6 | 6 |
| 5 | AB-101 Dose 4 | 2.5 × 10⁷ | 0.25 | 10 × 10⁷ | 6 | 6 |

Dose formulations were prepared freshly on each dosing day from frozen test article stock vials. The cell test article and the vehicle article were thawed by submerging in a water bath set at 37° C. Once thawed, the test article was diluted in the vehicle to reach the target cell numbers (within ±15%) to be used for dosing animals. A pilot pretest run was conducted to test the cell test article stability in the formulation during the bench top storage and its syringe compatibility.

The vehicle control or cell test article were administered to Groups 1-3 on Days 1 and 3 and then weekly starting on Day 12 (Days 1, 3, 12, 19, 26, 33, 40, 47, and 54). Cell test article was administered to Group 4 on Days 1 and 3 only, and to group 5 weekly throughout (Days 1, 8, 15, 22, 29, 36, 43, and 50). The animals were dosed via IV slow bolus injection (over a period of 30-40 seconds) via the tail vein. The dose levels for Groups 1-3 were 0, 0.1×10⁷ cells, and 0.5×10⁷ cells and for Groups 4 and 5 were 2.5×10⁷ cells and administered at a dose volume of 0.25 mL.

Following the first dose, animals in Group 4 exhibited decreased activity and/or skin cold to touch. These observations resolved prior to the next dose. Following Dose 2 (Day 3), animals in Group 4 exhibited clinical and/or veterinary observations of severely decreased activity, hunched posture, piloerection, partially closed eyes, paleness, loss of skin elasticity, skin cold to touch, and irregular respiration. Due to the severity of these findings, these animals were scheduled for early euthanasia on Day 3.

Observations of minor swelling (foot/limb) and/or impaired limb function and/or splayed limbs were noted in all groups, including vehicle control animals. These findings were present in most animals and were considered vehicle related but not adverse due to their minor nature and lack of painful response when palpitated.

Test article-related findings of tremors and unkempt appearance were noted in both Groups 3 and 5. Additional test article-related clinical and/or veterinary findings in Group 5 consisted of moderately decreased activity, ataxia, slow breathing, eyes partially/completely closed, skin cold to touch, and loss of skin elasticity. These findings in Group 5 were primarily noted following the 6th dose and the animals generally recovered by the next day. Following the 8th dose, animals in Group 5 exhibited severely decreased activity, dehydration, weakness, ataxia, partially closed eyes, irregular breathing, unkempt appearance, low carriage, splayed limbs, and skin cold to touch. Despite the noted severity of the findings following Dose 8, all animals recovered by the following day. The findings in Group 5 were considered adverse due to the increased severity of the observations with the increased number of doses administered. The findings in Group 3 were not considered adverse, due to the low rate of occurrence and minor nature of the observations. There were no test article-related findings in Group 2.

Conclusions. AB-101 was given as weekly injections for 8 weeks at dose levels ranging from 0.1×10⁷ to 2.5×10⁷ cells/dose/animal. Among different dose levels that were tested in DRF study, at a dose level of 2.5×10⁷ cells following the 8th dose, adverse clinical observations including severely decreased activity, dehydration, weakness, ataxia, partially closed eyes, irregular breathing, unkempt appearance, low carriage, splayed limbs, and skin cold to touch were noted. These findings were considered adverse, despite recovery, due to the increased severity and number of findings noted in these animals following the 8th dose. Non-adverse test article related changes were noted at dose levels of 0.5×10⁷ and 2.5×10⁷ cells. Non-adverse clinical observations including tremors, unkempt appearance, piloerection were noted at a dose level of 0.5×10⁷ cells. At a dose level of 2.5×10⁷ cells, non-adverse test article related changes such as decreased gain in body weights and changes in clinical pathology endpoints that included decreased red cell mass and reticulocytes, increased leukocytes and neutrophils were observed. No anatomic pathology findings were observed in animals in DRF study. In conclusion, the dose level of 2.5×10⁷ cells that was administered intravenously once weekly for 8 weeks to NSG mice was determined as the Maximum Tolerated Dose (MTD).

The objective of the GLP toxicity study was to evaluate acute and delayed toxicity of AB-101 in NSG mice after repeated intravenous dosing. Doses for GLP toxicity study were selected as per the results obtained from DRF study. In DRF study, at the dose of 2.5×10⁷ cells following the 8th dose, adverse clinical observations were noted. Thus, a cell number of 2×10⁷ was used as the highest dose in the GLP toxicity study. Mice were administered AB-101 once weekly for 8 weeks. Doses 0.5×10⁶ cells/animal and 2×10⁷ cells/animal used in GLP toxicity study provides sufficient safety margin with expected clinical dosing (0.5×10⁷ cells/animal corresponds to 14×10⁹ cells of equivalent human cell dose and 2×10⁷ cells/animal corresponds to 56×10⁹ cells of equivalent human cell dose for a patient of 70 kg). Mouse to human doses were scaled allometrically. The experimental design for the study is shown in Table 10.

TABLE 10
Experimental Design for Acute and Delayed Toxicity Study

| | | | | # NSG Mice |
|---|---|---|---|---|
| | | | # NSG Mice | (Recovery |
| Group | Test Article | Target Dose | (Main Study) | Study) |
| 1 | Vehicle | 0.25 mL | 20 (10 male, 10 | 26 (13 male, 13 |
| | | | female)^{a,b} | female)^{a,b,c} |
| 2 | AB-101 | 0.5 × 10⁷ cells/dose | 20 (10 male, 10 | 26 (13 male, 13 |
| | | in 0.25 mL | female)^{a,b} | female)^{a,b,c} |
| 3 | AB-101 | 2 × 10⁷ cells/dose | 20 (10 male, 10 | 26 (13 male, 13 |
| | | in 0.25 mL | female)^{a,b} | female)^{a,b,c} |

^{a}5/sex/group for hematology analysis, histology tissue collection
^{b}5/sex/group for clinical chemistry (minus electrolytes) analysis, histology tissue collection
^{c}Last surviving 3/sex/group for qPCR blood and tissue collection Day 78 (i.e. 4 weeks after last dose)

As in DRF study, dose formulations were prepared freshly on each dosing day from frozen test article stock vials.

The cell test article and vehicle control were administered by intravenous injection via the tail vein. The dose was administered by a slow bolus injection (over period of 30 to 40 seconds). Administration occurred 1× Weekly for 8 weeks.

Once weekly intravenous administration of AB-101 at dose levels of 0.5×10⁷ and 2×10⁷ viable cells, in mice, resulted in no test article related mortalities, changes in body weight, ophthalmology, clinical pathology, or anatomic pathology endpoints. Based on a lack of adverse findings, the No-Observed-Effect-Level (NOEL) was the dose level of 2×10⁷ viable cells.

### Example 7: Treatment of Lupus Nephritis with AB-101 and a B-Cell Depleting mAb

B cells are recognized as key mediators of SLE pathogenesis and anti-CD20 mAbs promote direct B-cell killing via antibody-dependent cellular cytotoxicity (ADCC) and induction of apoptosis. However, studies have shown that B cell depletion is incomplete in the tissues of certain patients following rituximab treatment, leading to pathogenic B cell escape and hindering effective reset of the immune system. NK cells from SLE patients have been shown to be reduced in number in peripheral blood, with reduced cytotoxicity as well as defective ADCC, which could lead to incomplete B cell depletion by rituximab. Various non-clinical studies indicated that AB-101 can potentiate anti-CD20-mediated ADCC against CD20-expressing malignant B-lymphocytes in vitro and in vivo and is not negatively impacted by glucocorticoids. Further, AB-101 has been shown to induce B cell apoptosis in combination with rituximab in PBMCs isolated from SLE patients. Initial clinical data with rituximab and AB-101 in subjects with advanced B-cell malignancies showed that this treatment approach is generally well tolerated and can induce deep responses in malignant B-cells disorders.

AB-101 is a non-engineered, allogeneic, off-the-shelf, cryopreserved cord blood-derived natural killer (NK)-cell therapy. Cord blood units (CBU's) are pre-selected for KIR-B haplotype and the natural high-affinity variant of CD16 (158V/V), which is associated with ADCC enhancement, resulting in a highly active NK cell product without the requirement for additional engineering.

AB-101 is a commercially scaled and cryopreserved allogeneic NK cell product in an infusion ready media. The starting material is a Food and Drug Administration (FDA) licensed cord blood unit with the following attributes: selected killer immunoglobulin-like receptor B (KIR B) haplotype: demonstrates a more active phenotype (Cooley, 2009); selected V/V CD16 polymorphism at F158: demonstrates higher affinity binding to mAb Fc domain for enhanced antibody-dependent cellular cytotoxicity [ADCC](Musolino, 2008).

In preclinical studies, AB-101 demonstrated direct, specific, and potent killing of multiple tumor cell lines in vitro and in vivo when combined with tumor-targeting monoclonal antibodies. AB-101 has also demonstrated the ability to secrete cytokines such as tumor necrosis factor alpha (TNFα) and interferon gamma (IFNγ) upon activation. AB-101 is currently being investigated in a Phase1/2 clinical trial as monotherapy and in combination with rituximab in subjects with r/r NHL of B-cell origin (ClinicalTrials.gov: NCT04673617) and in combination with AFM13 (a bispecific CD30/CD16 antibody designed to redirect and enhance natural killer (NK) cell-mediated antibody-dependent cellular cytotoxicity) in patients with r/r HL and CD30+PTCL (NCT05883449).

NK cells are unique in their ability to elicit rapid cytolytic responses without the need for antigen presentation or prior sensitization (Miller 2013; Malmberg 2017). This is accomplished in part through the integration of signals delivered from both inhibitory and activating NK cell receptors, and this combinatorial regulation of activity allows for the recognition of a broad range of malignant or virally infected cell types while avoiding improper targeting of healthy cells and tissues (Vivier 2008; Guillerey, Huntington, and Smyth 2016). In addition to direct cytotoxicity, NK cells can exert their effects through antibody-dependent cytotoxicity (ADCC), whereby NK cells bind to the fragment crystallizable (Fc) portion of tumor-targeting antibodies, which coat the target cell surface, and lyse the target cell through the release of cytotoxic factors (e.g. perform, granzymes).

In SLE, NK cell dysfunction includes defective ADCC (Kozlowski, 1982, Green, 2005). Anti-CD20 antibodies use ADCC as part of the mechanism of action to kill malignant B-cells in cancer (Manches. 2003, Boross, 2012) and to deplete pathogenic B-cells in SLE (Albert, 2008). As discussed above, treatment of SLE/LN patients with rituximab failed to demonstrate significant therapeutic efficacy and was associated with a variable degree of B-cell depletion (Rovin, 2012). Obinutuzumab treatment of SLE/LN patients demonstrated improved efficacy and more rapid, deep, and durable B-cell depletion compared to that observed with rituximab (Furie, 2020); however, not all patients responded thus indicating that there is still an area of unmet need. Replacement of endogenous SLE NK cells with an allogeneic NK cell product would provide competent effector cells to enhance the activity of anti-CD20 antibodies. AB-101 is combined with these antibodies to enhance B-cell depletion through ADCC. ADCC is mediated through the expression of Fcγ receptors (FcγR) on the surface ofNK cells, namely CD16 (or FcγRIII). CD16 is expressed on ≥80% of AB-101 making it an ideal cell therapy candidate for combination with anti-CD20 antibodies in SLE/LN.

Nonclinical pharmacology of AB-10l has been investigated in both in vitro and in vivo studies to evaluate cytotoxicity against tumor cell lines, biodistribution, efficacy, and safety.

Relevant to the ADCC mechanism of action, the cytotoxic activity of AB-101 was evaluated against B-cell lymphoma or leukemia cell lines, normal human peripheral blood mononuclear cells (PBMCs) and PBMCs isolated from SLE patients, in combination with anti-CD20 (rituximab and obinutuzumab) or anti-CD19 (tafasitamab) monoclonal antibodies. Tumor cells or PBMCs were incubated with or without antibodies at different effector (NK cell) to target (tumor cells or PBMCs) ratios (E:T ratios). Apoptotic (caspase positive) cells were quantified by flow cytometry.

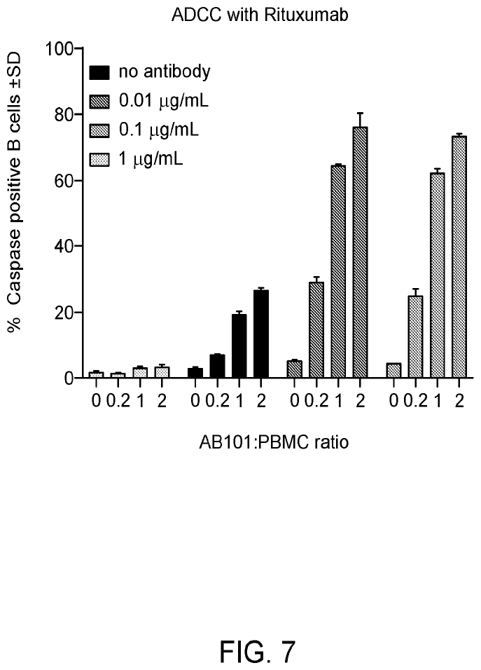

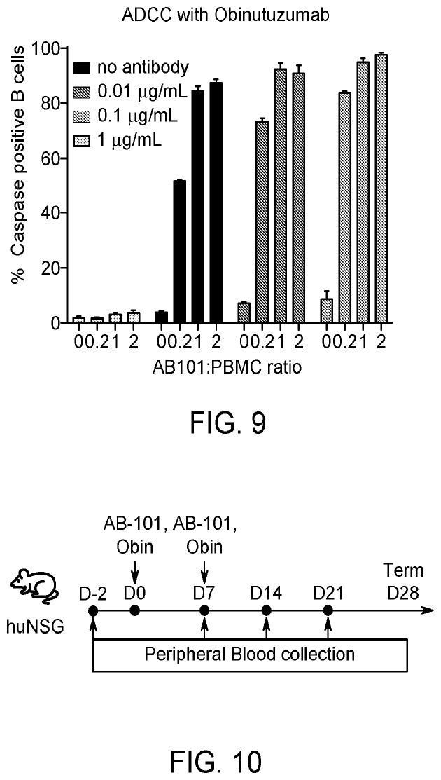

All three antibodies enhanced ADCC activity of AB-101 against B-cell lymphoma or leukemia cell lines. Cytotoxicity experiments utilizing normal human PBMCs co-cultured with AB-101 demonstrated enhanced AB-101-mediated B cell killing at different E:T ratios in the presence of anti-CD20 or anti-CD19 antibodies. Enhanced ADCC against SLE B cells was observed when AB-101 was combined with Rituximab (FIG. 7) or Obinutuzumab (FIG. 9) in an antibody concentration-dependent and an E:T ratio-dependent manner. The specificity of cell killing in the SLE PBMC sample was evaluated by examining the effects of the combination on SLE T-cells. Little to no off-target apoptosis was observed in this cell population in the presence of AB-101 and any of the three antibodies tested.

SLE patient PBMCs were isolated from peripheral blood and combined with thawed AB-101, with or without the anti-CD20 antibody, rituximab, for 4 hours. The percentage of caspase positive B cells was determined by flow cytometry. Data are expressed as the mean+/−SD of duplicate wells. Representative data is shown from one SLE patient sample.

The combination of AB-101 and a B-cell depleting mAb (e.g., rituximab or obinutuzumab) may be an effective means to cause substantial and deep B-cell depletion, as well as inhibiting replenishment of short-lived plasmablasts, allowing the immune system to reset in these patients. This has the potential to provide a meaningful and long-lasting clinical response in patients with advanced proliferative lupus nephritis.

Subjects may receive concomitant antihypertensive and antiproteinuric therapy with blockade of the renin-angiotensin system (e.g., angiotensin-converting enzyme [ACE] inhibitor or angiotension II receptor blocker.

### Example A

In this example, adult subjects with lupus nephritis Class III or IV either with or without the presence of Class V who relapsed or did not respond to previous standard of care treatment approaches are treated with AB-101 plus a B-cell depleting mAb (e.g., Rituximab) after a lymphodepletion regimen. Patients are administered AB-101 and Rituximab for up to a total of two treatment cycles, spaced 24-weeks apart.

Subjects will receive, at the beginning of each treatment cycle, 3 consecutive days (Days 1-3) of a lymphodepleting regimen to induce lymphocyte depletion and to create an optimal environment for expansion of AB-101 in vivo. Rituximab will be administered twice (spaced ~2 weeks apart) as an intravenous infusion of 1000 mg of each treatment cycle, after premedication with methylprednisolone to reduce the risk of infusion-related reactions. AB-101 will be dosed at one of three levels (Table 11) after the administration of the lymphodepleting regimen and after the first dose of Rituximab.

TABLE 11
Dose Levels and Dosing Days for AB-101

| Dosing | Day 6 | Day 9 | Day 13 | Day 16 | Day 20 |
|---|---|---|---|---|---|
| Dose Level −1 | 2 × 10⁹ cells | NA | 2 × 10⁹ cells | NA | 1 × 10⁹ cells |
| Dose Level 1 | 4 × 10⁹ cells | NA | 4 × 10⁹ cells | NA | 2 × 10⁹ cells |
| Dose Level 2 | 4 × 10⁹ cells | 2 × 10⁹ cells | 4 × 10⁹ cells | 2 × 10⁹ cells | NA |

The lymphodepletion regimen is a fludarabine and cyclophosphamide regimen in which fludarabine is administered at 30 mg/m2 on day 1, 2, and 3 at the beginning of each treatment cycle and cyclophosphamide is administered at 1000 mg/m2 on day 3 of each treatment cycle.

Rituximab is administered at 1000 mg on Day 2 and Day 13 of each treatment cycle.

The first dose of AB-101 is administered at least 48 hours after the last infusion of the lymphodepletion regimen for each treatment cycle, using the dosing scheme set forth in Table 11.

### Example B

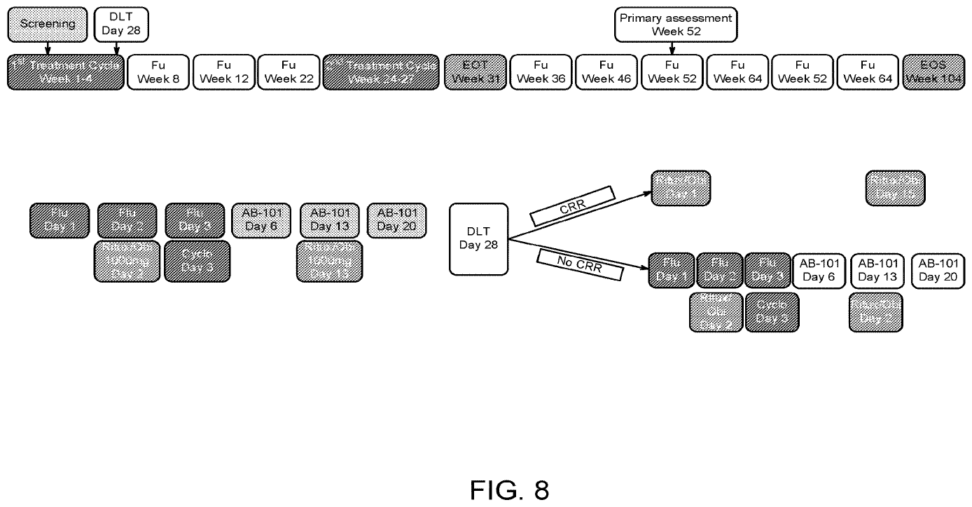

In this example, subjects adult subjects with relapsed/refractory lupus nephritis Class III or IV, with or without the presence of Class V, using the Common Terminology Criteria for Adverse Events (CTCAE) v.5.0 criteria are treated with AB-101 plus a B-cell depleting mAb (e.g., rituximab, obinutuzumab) after a lymphodepletion regimen. Patients are administered AB-101 and a B-cell depleting mAb for up to a total of two treatment cycles, spaced 24-weeks apart, as depicted in FIG. 8. Subjects with a complete renal response (CRR) at Week 22 will receive only the assigned mAb, if applicable. Subjects who have not achieved a complete renal response will receive the second cycle of the initial treatment regimen.

All subjects will receive, during their first treatment cycle, 3 consecutive days (Days 1-3) of a lymphodepleting regimen consisting of fludarabine (Day 1-3) and cyclophosphamide (Day 3) to induce lymphocyte depletion and to create an optimal environment for expansion of AB-101 in vivo. Rituximab or Obinutuzumab will be administered twice (spaced ~2 weeks apart) as an intravenous infusion of 1000 mg at each treatment cycle, after premedication with methylprednisolone to reduce the risk of infusion-related reactions. AB-101 will be dosed at two different schedules (see below) after the administration of the lymphodepleting regimen and after the first dose of Rituximab.

Seven cohorts of patients will be treated with AB-101 with or without a B-cell depleting antibody as follows:

TABLE 12
Cohorts and Dose Levels for AB-101

| Cohorts | Paired mAb | AB-101 Dose Levels |
|---|---|---|
| Monotherapy DL−1 | None | Dose Level −1 (5 × 10⁸ cells) |
| Monotherapy DL1 | None | Dose Level 1: 1 × 10⁹ cells |
| Monotherapy DL2# | None | Dose Level 1: 4 × 10⁹ cells |
| Rituxmab DL1 | Rituximab 1,000 mg/dose | Dose Level 1: 1 × 10⁹ cells |
| Rituximab DL2 | Rituximab 1,000 mg/dose | Dose Level 1: 4 × 10⁹ cells |
| Obinutuzumab DL1 | Obinutuzumab 1,000 mg/dose | Dose Level 1: 1 × 10⁹ cells |
| Obinutuzumab DL2 | Obinutuzumab 1,000 mg/dose | Dose Level 1: 4 × 10⁹ cells |

The Monotherapy DL1 cohort subjects will receive AB-101 as monotherapy on Days 6, 13 and 20 of Cycle 1.

Fludarabine (30 mg/m²) is administered on Day 1, Day 2 and Day 3 at the beginning of each treatment cycle; dose adjustment due to renal insufficiency or other medical conditions may apply. Cyclophosphamide (1000 mg/m²) is administered on Day 3 of each treatment cycle. AB-101 is administered as an IV infusion via gravity at a rate of 5-10 mL per minute on days 6, 13, and 20 of each treatment cycle. Patients may be pre-treated with one or more of: acetaminophen (500 to 1,000 mg given orally), diphenhydramine 12.5 to 25 mg IV or 25 mg given orally (e.g., when needed to treat hypersensitivity reactions to AB-101), and/or a second-generation oral antihistamine (e.g., cetirizine, fexofenadine, or loratadine).

The first dose of AB-101 in each cycle should be administered at least 48 h after the last infusion of the lymphodepletion (LD) regimen.

Rituximab or Obinutuzumab (1000 mg, if administered) is administered via infusion (at 50-400 mg/hour) on day 1 and day 15 of each treatment cycle. The patient may be pre-treated with one or more of a corticosteroid (prednisone, prednisolone, dexamethasone, or methylprednisolone), analgesic/antipyretic, and/or antihistamine, for example as outlined in Table 13, e.g., to reduce the incidence and severity of infusion-related-reactions (IRRs). The patient may also be pre-treated as outlined in Table 14. The patient's dose may also be adjusted as outlined in Table 14.

TABLE 13
Example of Pre-Medication Prior to Administration

| | Patients Requiring | | |
|---|---|---|---|
| Timepoint | Pre-medication | Pre-medication | Administration |
| Day 2 Cycle 1 | All patients | Oral corticosteroid ^{a} | Administer ≥1 hour prior to |
| | | | obinutuzumab infusion |
| | All patients | Oral analgesic/anti- | Administer ≥30 minutes |
| | | pyretic ^{b} | prior to obinutuzumab |
| | | Antihistamine drug ^{c} | infusion |
| | Patients at risk for TLS | Allopurinol or suitable | Administer prior to |
| | (e.g.. because of bulky | alternative such as | obinutuzumab infusion |
| | disease or renal | rasburicase, along with | |
| | impairment (creatinine | adequate hydration | |
| | clearance <70 mL/min) | | |

IRR = infusion-related reaction; TLS = tumor lysis syndrome.
^{a} Treat with 100 mg of prednisone or prednisolone (as an alternative: 20 mg of dexamethasone, or 80 mg of methylprednisolone may be administered).
^{b} For example, 1000 mg of acetaminophen/paracetamol.
^{c} For example, 50 mg of diphenhydramine.

TABLE 14
Example of Pre-Medication Prior to Administration
and Optional Dose Modifications

| | | Premedication | | Optional |
|---|---|---|---|---|
| | Patients | (30-60 min | | AB-101 |
| AB-101 | requiring | before NK | | Dose |
| infusions | premedication | cell infusion) | Administration | Modification |
| 1^{st} Infusion | All patients | 650-1,000 mg | At least 30 | |
| | | acetaminophen | minutes before | |
| | | antihistamine (e.g., | AB-101 | |
| | | 25-50 mg | infusion. | |
| | | diphenhydramine) | | |
| 2^{nd} Infusion and | All patients | 650-1,000 mg | At least 30 | |
| all subsequent | | acetaminophen | minutes before | |
| infusions | | | AB-101 | |
| | | | infusion. | |
| | Patients with | 650-1,000 mg | At least 30 | |
| | Grade 1-2 | acetaminophen | minutes before | |
| | IRR, CRS or | antihistamine | AB-101 | |
| | other Immune- | (e.g., 50 mg | infusion. | |
| | mediated | diphenhydramine) | | |
| | events with the | | | |
| | previous | | | |
| | infusion | | | |
| | Patients with a | 650-1,000 mg | At least 30 | Dose |
| | Grade 3 IRR, | acetaminophen | minutes before | reduction |
| | CRS or other | | AB-101 | by 50% |
| | Immune- | | infusion. | |
| | mediated | antihistamine | At least 30 | |
| | events with the | (e.g., 50 mg | minutes before | |
| | previous | diphenhydramine) | AB-101 | |
| | infusion | | infusion. | |
| | | Intravenous | At least 60 | |
| | | glucocorticoid: 20 | minutes before | |
| | | mg dexamethasone | AB-101 | |
| | | or 80 mg | infusion. | |
| | | methylprednisolone | | |

### Example 8: Treatment of Autoimmune Disorders with NK Cells and a B-Cell Depleting mAb

This study will evaluate the safety and activity of allogenic NK cells in combination with a B-cell depleting antibody (Rituximab) in adult subjects with one of the following disease conditions who have failed prior treatment approaches and are considered ‘resistant/refractory’ by the Investigator using the clinical indication specific disease criteria (below): Rheumatoid Arthritis (RA); Pemphigus vulgaris (PV); Granulomatosis with polyangiitis (GPA)/microscopic polyangiitis (MPA); and Systemic Lupus Erythematosus (SLE).

### Disease-Specific Inclusion Criteria

### Rheumatoid Arthritis:

i. Documented diagnosis of RA, meeting the 2010 American College of Rheumatology (ACR)/European League Against Rheumatism (EULAR) classification criteria for RA (Kay, 2012).
ii. Have had prior treatment with a biologic disease-modifying anti-rheumatic drugs (bDMARD e.g., Infliximab, Rituximab, Etanercept, Tocilizumab)) and/or a targeted synthetic disease-modifying anti-rheumatic drugs (tsDMARD e.g., baricitinib, tofacitinib)) and were deemed refractory by either:
   a. In the opinion of the Investigator, there was a lack of benefit to at least two bDMARDs or one bDMARD and one tsDMARDs.
   b. Lack of benefit may include inadequate improvement in joint counts, physical function, or disease activity. Intolerance to at least two lines of prior therapy, including bDMARDs and/or tsDMARDs.
iii. Minimum of 6 swollen joint counts (SJC) and 6 tender joint counts (TJC).
   Pemphigus vulgaris:
i. Confirmed diagnosis of pemphigus vulgaris with active lesions
ii. Positive for anti-desmoglein Dsg1 or Dsg3.
iii. Pemphigus Disease Area Index score (Shimizu, 2014) of >80%.
   Granulomatosis with Polyangiitis (GPA)/Microscopic Polyangiitis (MPA):
i. Clinical diagnosis of granulomatosis with polyangiitis (GPA) or microscopic polyangiitis (MPA).
ii. Have ≥1 “major” item, or ≥3 “other” items, or ≥2 renal items on the Birmingham Vasculitis Activity Score Version 3 (BVASv3).

### Systemic Lupus Erythematous:

i. Diagnosis of SLE according to the 2019 European League Against Rheumatism/American College of Rheumatology (EULAR/ACR) Classification Criteria.
ii. Total systemic lupus erythematosus disease activity index (SLEDAI-2K) total score of ≥6 at screening.
iii. Positive for anti-double-stranded deoxyribonucleic acid (dsDNA) antibodies.

Patients are administered: 1) NK cells; and 2) a B-cell depleting antibody (Rituximab). The NK cells administered (AB-101) are a cryopreserved, infusion-ready suspension cell therapy comprised of ex-vivo expanded allogenic cord blood-derived NK cells that have not been genetically modified, made from Food and Drug Administration (FDA)-licensed cord blood units pre-selected for a) V/V CD16 polymorphism at F158 for enhanced antibody-dependent cellular cytotoxicity (ADCC) and b) killer immunoglobulin-like receptor B (KIR-B) haplotype.

All subjects will receive one treatment cycle. The treatment cycle will be initiated with 3 consecutive days (Days 1 to 3) of a lymphodepleting regimen consisting of fludarabine (25 mg/m² on Day 1, Day 2 and Day 3 (reduced or eliminated for subjects with impairment of renal function)) and cyclophosphamide (1000 mg/m² on Day 3). Rituximab will be administered twice (1000 mg on Day 2 and Day 13) as an intravenous infusion, after premedication with methylprednisolone to reduce the risk of infusion-related reactions. NK cells will be dosed at 1B (1×10\hat{ }9) cells on Day 6, Day 13, and Day 20. The first dose of NK cells should be administered at least 48 h (and up to 7 days) after the last infusion of the lymphodepletion regimen. The infusion schedule, cycle duration, and study drug administration schedule may be changed based on emerging safety, pharmacokinetic, and biomarker data as determined by the Investigator.

### Study Assessments/Endpoints

Safety: Safety will be assessed throughout the study conduct by monitoring Adverse events (AEs), concomitant medications, physical examinations, vital signs, and laboratory test findings. The severity of AEs will be graded according to the CTCAE version 5.0, except for the adverse events noted: Cytokine Release Syndrome (CRS): severity of CRS events will be assessed by American Society for Transplantation and Cellular Therapy (ASTCT) grading (Lee, 2019): Immune Effector Cell-associated Neurotoxicity Syndrome (ICANS); severity of ICANS events will be assessed by American Society for Transplantation and Cellular Therapy (ASTCT) grading (Lee, 2019); Graft-versus-Host-Disease (GvHD); diagnosis and grading of GvHD should follow the Mount Sinai Acute GvHD International Consortium (MAGIC) criteria (Harris, 2016)

Primary Activity Endpoint: RA: Change From Baseline in DAS28 at Week 12 and 24: PV: Change from baseline in the Pemphigus Disease Area Index (PDAI) at Week 12 and 24: GPA/MPA: Change from baseline in the Birmingham Vasculitis Activity Score at Week 12 and 24; SLE: Change from baseline in the SLE Disease Activity Index (SLEDAI) at Week 12 and 24:

Exploratory Endpoints (change from baseline at Week 12 and 24). Rheumatoid Arthritis: Change in Clinical Disease Activity Index (CDAI); Change in participant response assessed by EULAR criteria. Pemphigus vulgaris: Time to and number of disease flares; Time to initial complete remission, evaluated by PDAI; Change in health related QoL (by Dermatology Life Quality Index (DLQI); Blood DSG 1 and 3 levels. Granulomatosis with polyangiitis (GPA) and microscopic polyangiitis (MPA): Time to and number of disease flares: Time to initial complete remission; Change in health related QoL (by Dermatology Life Quality Index (DLQI). Systemic Lupus Erythematosus (SLE): Change is Systemic Lupus Activity Measure (SLAM): C3 and C4 complement levels.

### Example 9: SLE Donor Cell Characterization

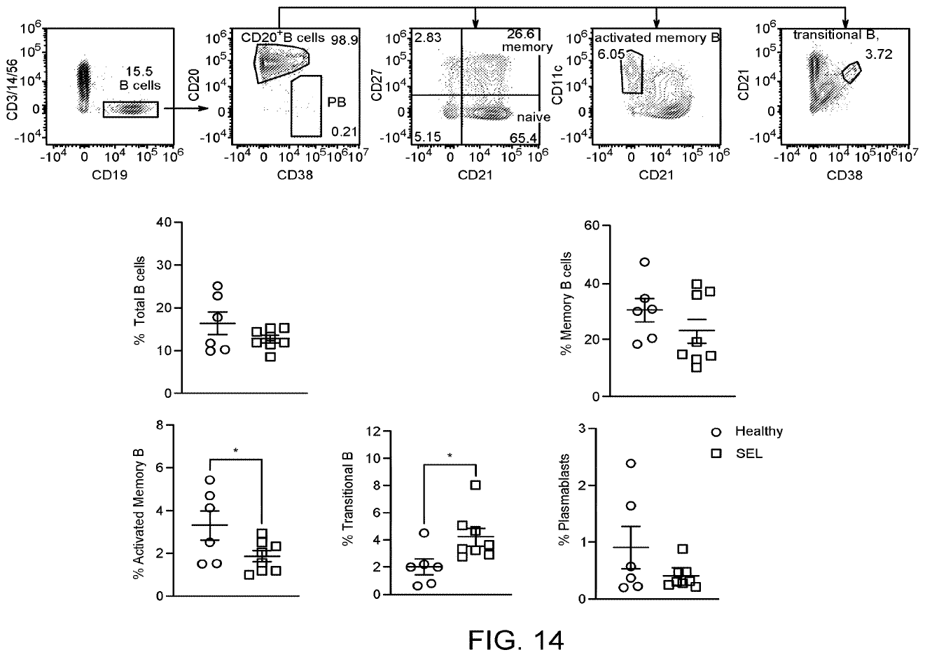

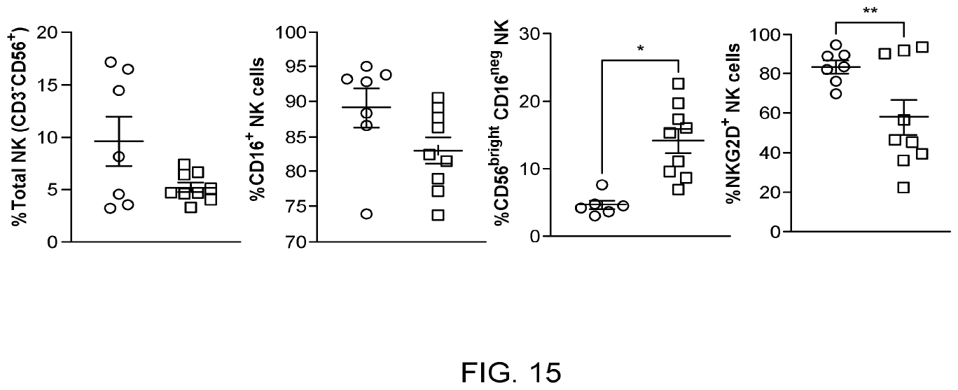

SLE and healthy donor B cells and NK cells were characterized. As shown in FIG. 14, SLE donors had altered B cell subsets with increased transitional B and decreased activated memory B cells. As shown in FIG. 15, SLE donors had reduced total NK, CD16+ and NKG2D but increased CD56^{bright} CD16^{neg} 8 NK cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a patient suffering from an autoimmune disorder, the method comprising administering a population of natural killer cells (NK cells) and an antibody targeted to an immune cell, wherein the NK cells are allogenic to the patient.

2. The method of claim 1, wherein the immune cell is implicated in an autoimmune reaction.

3. The method of claim 1 or claim 2, wherein the immune cell is a B cell.

4. The method of any one of claims 1 to 3, wherein the antibody is a B-cell depleting antibody.

5. The method of any one of claims 1 to 4, wherein the antibody is an antibody targeted to human CD19 and/or human CD20.

6. The method of any one of claims 1 to 5, wherein the NK cells are KIR-B haplotype and homozygous for a CD16 158V polymorphism.

7. The method of any one of claims 1 to 6, wherein the autoimmune disease is selected from, Acromegaly, Acquired aplastic anemia, Acquired hemophilia, Primary Agammaglobulinemia, Alopecia areata, Ankylosing spondylitis (AS), Anti-NMDA receptor encephalitis, Antiphospholipid syndrome (APS)|catastrophic antiphospholipid syndrome (CAPS)/Asherson's syndrome, Arteriosclerosis, Autoimmune Addison's disease (AAD), Autoimmune autonomic ganglionopathy (AAG)/autoimmune dysautonomia|autoimmune gastrointestinal dysmotility (AGID), Autoimmune encephalitis|acute disseminated encephalomyelitis (ADEM), Autoimmune gastritis, Autoimmune hemolytic anemia (AIHA), Autoimmune hepatitis (AIH), Autoimmune hvperlipidemia, Autoimmune hypophysitis, Autoimmune inner ear disease (AIED), Autoimmune lymphoproliferative syndrome (ALPS), Autoimmune myelofibrosis, Autoimmune myocarditis, Autoimmune oophoritis, Autoimmune pancreatitis (AIP), Autoimmune polyglandular syndromes, types I, II, & III (APS type 1, APS type 2, APS type 3, APECED). Autoimmune progesterone dermatitis, Autoimmune retinopathy (AIR), Autoimmune sudden sensorineural hearing loss (SNHL), Balo disease, Behçet's disease, Birdshot chorioretinopathy/birdshot uveitis, Bullous pemphigoid, Castleman disease, Celiac disease, Chagas disease, Chronic inflammatory demyelinating polyneuropathy (CIDP), Chronic urticaria (CU), Churg-Strauss syndrome/eosinophilic granulomatosis with polyangiitis (EGPA), Cogan's syndrome, Cold agglutinin disease, CREST syndrome|limited cutaneous systemic sclerosis, Crohn's disease (CD), Cronkhite-Canada syndrome (CSS), Cryptogenic organizing pneumonia (COP), Dermatitis herpetiformis, Dermatomyositis, Type 1 Diabetes, Discoid lupus, Dressler's syndrome/postmyocardial infarction/postpericardiotomy syndrome, Eczema/Atopic Dermatitis, Endometriosis, Eosinophilic esophagitis, Eosinophilic fasciitis, Erythema nodosum, Essential mixed cryoglobulinemia, Evans syndrome, Fibrosing alveolitis/Idiopathic pulmonary fibrosis (IPF), Giant cell arteritis/temporal arteritis/Horton's disease, Giant Cell Myocarditis, Glomerulonephritis, Goodpasture's syndrome/anti-GBM/anti-TBM disease, Granulomatosis with polyangiitis (GPA)/Wegener's granulomatosis, Graves disease/thyroid eye disease, Guillain-Barre syndrome (GBS), Hashimoto's thyroiditis/chronic lymphocytic thyroiditis/autoimmune thyroiditis, Henoch-Schönlein purpura/IgA vasculitis, Hidradenitis suppurativa, Hurst's disease/acute hemorrhagic leukoencephalitis (AHLE), Hypogammaglobulinemia, IgA nephropathy/Berger's disease, Immune-mediated necrotizing myopathy (IMNM), Immune thrombocytopenia (ITP)/autoimmune thrombocytopenic purpura/autoimmune thrombocytopenia, Inclusion body myositis, IgG4-related sclerosing disease (ISD), Interstitial cystitis, Juvenile idiopathic arthritis/Adult-onset Still's disease, Juvenile polymyositis|Juvenile dermatomyositis|juvenile myositis, Kawasaki disease, Lambert-Eaton myasthenic syndrome (LEMS), Leukocytoclastic vasculitis, Lichen planus, Lichen sclerosus, Ligneous conjunctivitis, Linear IgA disease (LAD)|linear IgA bullous dermatosis (LABD), Lupus nephritis, Lyme disease/chronic Lyme disease/post-treatment Lyme disease syndrome (PTLDS), Lymphocytic colitis/microscopic colitis, Lymphocytic hypophystitis/autoimmune hypophystitis, Meniere's disease, Microscopic polyangiitis (MPA)/ANCA-associated vasculitis, Mixed connective tissue disease (MCTD), Mooren's ulcer, Mucha-Habermann disease, Multifocal motor neuropathy, Multiple sclerosis (MS), Myalgic encephalomyelitis (ME)/Chronic fatigue syndrome (CFS), Myasthenia gravis (MG), Narcolepsy, Neuromyelitis Optica/Devic's disease, Ocular cicatricial pemphigoid, Opsoclonus-myoclonus syndrome (OMS), Palindromic rheumatism, Paraneoplastic cerebellar degeneration, Paraneoplastic pemphigus, Parry-Romberg syndrome (PRS)/Hemifacial atrophy (HFA)/Progressive facial hemiatrophy, Paroxysmal nocturnal hemoglobinuria (PNH), Peripheral uveitis/pars planitis, PANS/PANDAS, Parsonage-Turner syndrome, Pemphigus gestationis/herpes gestationis, Pemphigus foliaceus, Pemphigus vulgaris, Pemicious anemia, POEMS syndrome, Polyarteritis nodosa, Polymyalgia rheumatica, Polymyositis, Postural orthostatic tachycardia syndrome (POTS), Primary biliary cirrhosis (PBC)/primary biliary cholangitis, Primary sclerosing cholangitis (PSC), Psoriasis, Palmoplantar Pustulosis, Psoriatic arthritis, Pulmonary fibrosis, idiopathic (IPF), Pure red cell aplasia (PRCA), Poderma gangrenosum, Rasmussen's encephalitis, Raynaud's syndrome/phenomenon, Reactive arthritis/Reiter's syndrome, Reflex sympathetic dystrophy syndrome (RSD)/Complex regional pain syndrome (CRPS), Relapsing polychondritis, Restless leg syndrome (RLS)/Willis-Ekbom disease, Rheumatic fever, Rheumatoid arthritis, Sarcoidosis, Schmidt syndrome/autoimmune polyendocrine syndrome type II, Scleritis, Scleroderma, Sclerosing Mesenteritis/Mesenteric Panniculitis, Serpiginous choroidopathy, Sjögren's syndrome, Stiff person syndrome (SPS), Small fiber sensory neuropathy, Systemic lupus erythematosus (SLE), Subacute bacterial endocarditis (SBE), Subacute cutaneous lupus, Susac syndrome, Sydenham's chorea, Sympathetic ophthalmia, Takayasu's arteritis (vasculitis), Testicular autoimmunity (vasculitis, orchitis), Tolosa-Hunt syndrome, Transverse Myelitis (TM), Tubulointerstitial nephritis uveitis syndrome (TINU), Ulcerative colitis (UC), Undifferentiated connective tissue disease (UCTD), Uveitis|anterior/intermediate/posterior, Vasculitis, VEXAS Syndrome, Vitiligo, Vogt-Koyanagi-Harada syndrome (VKH), and combinations thereof.

8. The method of claim 7, wherein autoimmune disorder is Systemic lupus erythematosus (SLE).

9. The method of any one of the preceding claims, wherein the patient has lupus nephritis.

10. The method of claim 7, wherein autoimmune disorder is rheumatoid arthritis (RA).

11. The method of claim 7, wherein autoimmune disorder is pemphigus vulgaris (PV)

12. The method of claim 7, wherein autoimmune disorder is granulomatosis with polyangiitis (GPA).

13. The method of claim 7, wherein autoimmune disorder is microscopic polyangiitis (MPA).

14. The method any one of the foregoing claims, wherein the patient has relapsed after treatment with an anti-CD19 and/or anti-CD20 antibody.

15. The method of any one of the foregoing claims, wherein the patient has experienced disease progression after treatment with autologous stem cell transplant or chimeric antigen receptor T-cell therapy (CAR-T).

16. The method of any one of the foregoing claims, wherein the patient is administered 1×10⁸ to 1×10¹⁰ NK cells.

17. The method of any one of the foregoing claims, wherein the patient is administered 1×10⁹ to 8×10⁹ NK cells.

18. The method of any one of the foregoing claims, wherein the patient is administered 4×10⁸, 1×10⁹, 4×10⁹, or 8×10⁹ NK cells.

19. The method of any one of the foregoing claims, wherein the patient is administered 5×10\hat{ }8, 1×10\hat{ }9, or 4×10\hat{ }9 NK cells.

20. The method of any of the forgoing claims, wherein the antibody is selected from Table 1, Table 2 or Table 3.

21. The method of claim 20, wherein the antibody is rituximab, obinutuzumab, or tafasitamab.

22. The method of claim 20, wherein the antibody is rituximab.

23. The method of claim 20, wherein the antibody is obinutuzumab.

24. The method of claim 20, wherein the antibody is tafasitamab.

25. The method of any of the forgoing claims, wherein the patient is subjected to lymphodepleting chemotherapy prior to treatment.

26. The method of claim 25, wherein the lymphodepleting chemotherapy is non-myeloablative chemotherapy.

27. The method of claim 25 or claim 26, wherein the lymphodepleting chemotherapy comprises treatment with at least one of cyclophosphamide and fludarabine.

28. The method of claim 27, wherein the lymphodepleting chemotherapy comprises treatment with cyclophosphamide and fludarabine.

29. The method of any one of claims 27-28, wherein the cyclophosphamide is administered between 100 and 500 mg/m²/day.

30. The method of claim 29, wherein the cyclophosphamide is administered at 250 or 300 mg/m²/day.

31. The method of claim 29, wherein the cyclophosphamide is administered at 500 mg/m²/day.

32. The method of any one of claims 27-31, wherein the fludarabine is administered between 10 and 50 mg/m²/day.

33. The method of claim 31, wherein the fludarabine is administered 30 mg/m²/day.

34. The method of any of the forgoing claims further comprising administering IL-2.

35. The method of claim 34, wherein the patient is administered 1×10⁶ IU/m² of IL-2.

36. The method of claim 34, wherein the patient is administered 6 million IU of IL-2.

37. The method of any one of claims 34-36, wherein administration of IL-2 occurs within 1-4 hrs of administration of the NK cells.

38. The method of any of the forgoing claims wherein the administration of the NK cells and the antibody occurs weekly.

39. The method of any of the forgoing claims wherein the NK cells and the antibody are administered weekly for 4 to 8 weeks.

40. The method of any one of the foregoing claims, wherein lymphodepletion occurs on days 1, 2, and 3 of a treatment cycle.

41. The method of any one of the foregoing claims, wherein the NK cells are administered on days 6, 13, and 20 or 6, 9, 13 and 16 of a treatment cycle.

42. The method of any one of the foregoing claims, wherein the NK cells are administered at or at about 2 billion or 4 billion cells per administration.

43. The method of any one of the foregoing claims, wherein, if administered, the NK cells are administered at or at about 4 billion cells on days 6, and 13.

44. The method of any one of the foregoing claims, wherein, if administered, the NK cells are administered at or at about 2 billion cells on days 9, 16, and 20.

45. The method of any one of the foregoing claims, wherein the NK cells are administered at or at about 5×10\hat{ }8, 1×10\hat{ }9, or 4×10\hat{ }9 NK cells on days 6, 13, and 20.

46. The method of any one of the foregoing claims, wherein the antibody is administered on days 2 and 13 of a treatment cycle.

47. The method of any of the forgoing claims wherein the administration of the NK cells occurs weekly and the administration of the antibody occurs every other week.

48. The method of any of the forgoing claims, wherein the NK cells are not genetically modified.

49. The method of any of the forgoing claims, wherein at least 70% of the NK cells are CD56+ and CD16+.

50. The method of any of the forgoing claims, wherein at least 85% of the NK cells are CD56+ and CD3−.

51. The method of any of the forgoing claims, wherein 1% or less of the NK cells are CD3+, 1% or less of the NK cells are CD19+ and 1% or less of the NK cells are CD14+.

52. The method of any of the forgoing claims wherein each administration of NK cells is administration of 1×10⁹ to 5×10⁹ NK cells.

53. The method of claim 34, wherein each administration of NK cells is administration of 1×10⁹ to 5×10⁹ NK cells.

54. The method of any of the forgoing claims wherein the patient receives a dose of the CD20 targeted antibody before the first dose of NK cells.

55. The method of any of the forgoing claims, wherein the expanded natural killer cells are expanded umbilical cord blood natural killer cells.

56. The method of any of the forgoing claims, wherein the population of expanded natural killer cells comprises at least 60%, e.g., at least 70%, at least 80%, at least 90% at least 95%, at least 99%, or 100% CD16+ cells.

57. The method of any of the forgoing claims, wherein the population of expanded natural killer cells comprises at least 60%, e.g., at least 70%, at least 80%, at least 90% at least 95%, at least 99%, or 100% NKG2D+ cells.

58. The method of any of the forgoing claims, wherein the population of expanded natural killer cells comprises at least 60%, e.g., at least 70%, at least 80%, at least 90% at least 95%, at least 99%, or 100% NKp46+ cells.

59. The method of any of the forgoing claims, wherein the population of expanded natural killer cells comprises at least 60%, e.g., at least 70%, at least 80%, at least 90% at least 95%, at least 99%, or 100% NKp30+ cells.

60. The method of any of the forgoing claims, wherein the population of expanded natural killer cells comprises at least 60%, e.g., at least 70%, at least 80%, at least 90% at least 95%, at least 99%, or 100% DNAM-1+ cells.

61. The method of any of the forgoing claims, wherein the population of expanded natural killer cells comprises at least 60%, e.g., at least 70%, at least 80%, at least 90% at least 95%, at least 99%, or 100% NKp44+ cells.

62. The method of any of the forgoing claims, wherein the population of expanded natural killer cells comprises less than 20%, e.g., 10% or less, 5% or less, 1% or less, 0.5% or less, or 0% CD3+ cells.

63. The method of any of the forgoing claims, wherein the population of expanded natural killer cells comprises less than 20% or less, e.g., 10% or less, 5% or less, 1% or less, 0.5% or less, or 0% CD14+ cells.

64. The method of any of the forgoing claims, wherein the population of expanded natural killer cells comprises less than 20% or less, e.g., 10% or less, 5% or less, 1% or less, 0.5% or less, or 0% CD19+ cells.

65. The method of any of the forgoing claims, wherein the population of expanded natural killer cells comprises less than 20% or less, e.g., 10% or less, 5% or less, 1% or less, 0.5% or less, or 0% CD38+ cells.

66. The method of any of the forgoing claims, wherein the natural killer cells do not comprise a CD16 transgene.

67. The method of any of the forgoing claims, wherein the natural killer cells do not express an exogenous CD16 protein.

68. The method of any of the forgoing claims, wherein the expanded natural killer cells are not genetically engineered.

69. The method of any of the forgoing claims, wherein the expanded natural killer cells are derived from the same umbilical cord blood donor.

70. The method of any of the forgoing claims, wherein the population of NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, I trillion, 2 trillion, 3 trillion, 4 trillion, 5 trillion, 6 trillion, 7 trillion, 8 trillion, 9 trillion, or 10 trillion expanded natural killer cells.

71. The method of any of the forgoing claims, wherein the population of NK cells is produced by a method comprising:
(a) obtaining seed cells comprising natural killer cells from umbilical cord blood:
(b) depleting the seed cells of CD3+ cells;
(c) expanding the natural killer cells by culturing the depleted seed cells with a first plurality of Hut78 cells engineered to express a membrane bound IL-21, a mutated TNFα, and a 4-1BBL gene to produce expanded natural killer cells,
thereby producing the population of expanded natural killer cells.

72. The method of any of the forgoing claims, wherein the population of NK cells is produced by a method comprising:
(a) obtaining seed cells comprising natural killer cells from umbilical cord blood;
(b) depleting the seed cells of CD3+ cells;
(c) expanding the natural killer cells by culturing the depleted seed cells with a first plurality of Hut78 cells engineered to express a membrane bound IL-21, a mutated TNFα, and a 4-1BBL gene to produce a master cell bank population of expanded natural killer cells; and
(d) expanding the master cell bank population of expanded natural killer cells by culturing with a second plurality of Hut78 cells engineered to express a membrane bound IL-21, a mutated TNFα, and a 4-1BBL gene to produce expanded natural killer cells;
thereby producing the population of expanded natural killer cells.

73. The method of claim 71 or claim 72, wherein the population of NK cells is produced by a method further comprising, after step (c),
(i) freezing the master cell bank population of expanded natural killer cells in a plurality of containers; and
(ii) thawing a container comprising an aliquot of the master cell bank population of expanded natural killer cells,
wherein expanding the master cell bank population of expanded natural killer cells in step (d) comprises expanding the aliquot of the master cell bank population of expanded natural killer cells.

74. The method of any one of claims 71 to 73, wherein the umbilical cord blood is from a donor with the KIR-B haplotype and homozygous for the CD16 158V polymorphism.

75. The method of any one of claims 71-74, wherein the population of NK cells is produced by a method comprising expanding the natural killer cells from umbilical cord blood at least 10.000 fold, e.g., 15,000 fold, 20,000 fold, 25,000 fold, 30.000 fold, 35,000 fold, 40,000 fold, 45,000 fold, 50,000 fold, 55,000 fold, 60,000 fold, 65,000 fold, or 70,000 fold.

76. The method of any one of claims 71-75, wherein the population of expanded natural killer cells is not enriched or sorted after expansion.

77. The method of any one of claims 71-76, wherein the percentage of NK cells expressing CD16 in the population of expanded natural killer cells is the same or higher than the percentage of natural killer cells in the seed cells from umbilical cord blood.

78. The method of any one of claims 71-77, wherein the percentage of NK cells expressing NKG2D in the population of expanded natural killer cells is the same or higher than the percentage of natural killer cells in the seed cells from umbilical cord blood.

79. The method of any one of claims 71-78, wherein the percentage of NK cells expressing NKp30 in the population of expanded natural killer cells is the same or higher than the percentage of natural killer cells in the seed cells from umbilical cord blood.

80. The method of any one of claims 71-79, wherein the percentage of NK cells expressing NKp44 in the population of expanded natural killer cells is the same or higher than the percentage of natural killer cells in the seed cells from umbilical cord blood.

81. The method of any one of claims 71-80, wherein the percentage of NK cells expressing NKp46 in the population of expanded natural killer cells is the same or higher than the percentage of natural killer cells in the seed cells from umbilical cord blood.

82. The method of any one of claims 71-81, wherein the percentage of NK cells expressing DNAM-1 in the population of expanded natural killer cells is the same or higher than the percentage of natural killer cells in the seed cells from umbilical cord blood.

83. A method for treating systemic lupus erythematosus (SLE), the method comprising:
administering a population of natural killer cells (NK cells) and an antibody targeted to a B cell to a patient diagnosed with SLE, wherein the NK cells are allogenic to the patient, wherein the NK cells are KIR-B haplotype and homozygous for a CD16 158V polymorphism.

84. The method of claim 83, wherein the patient has lupus nephritis (LN).

85. The method of claim 83 or 84, wherein the antibody is an anti-CD19 antibody and/or an anti-CD20 antibody.

86. The method of claim 85, wherein the antibody is rituximab, obinutuzumab, or tafasitamab.

87. The method of claim 85, wherein the antibody is rituximab.

88. The method of any one of claims 83 to 87, wherein the patient is administered a population of from 1×10\hat{ }9-5×10\hat{ }9 NK cells.

89. The method of claim 88, wherein the patient is administered a population of 2×10\hat{ }9 NK cells, or thereabout.

90. The method of claim 88, wherein the patient is administered a population of 4×10\hat{ }9 NK cells, or thereabout.

91. The method of claim 88, wherein the patient is administered a population of 5×10\hat{ }8 NK cells, or thereabout.

92. The method of claim 88, wherein the patient is administered a population of 1×10\hat{ }9 NK cells, or thereabout.

93. The method of any one of claims 83-90, wherein the patient is administered from 500 to 1500 mg of the antibody, or thereabout.

94. The method of claim 93, wherein the patient is administered 100 mg of the antibody, or thereabout.

95. The method of any one of claims 83-94, wherein the patient is administered lymphodepleting chemotherapy prior to administration of the NK cells.

96. The method of claim 95, wherein the lymphodepleting chemotherapy is non-myeloablative chemotherapy.

97. The method of claim 95 or claim 96, wherein the lymphodepleting chemotherapy comprises treatment with at least one of cyclophosphamide and fludarabine.

98. The method of claim 97, wherein the lymphodepleting chemotherapy comprises treatment with cyclophosphamide and fludarabine.

99. The method of any one of claims 97-98, wherein the cyclophosphamide is administered between 100 and 1500 mg/m2/day.

100. The method of claim 99, wherein the cyclophosphamide is administered at 500 or 1000 mg/m2/day.

101. The method of claim 99, wherein the cyclophosphamide is administered at 1000 mg/m2/day.

102. The method of any one of claims 97-101, wherein the fludarabine is administered between 10 and 50 mg/m2/day.

103. The method of claim 102, wherein the fludarabine is administered 30 mg/m2/day.

104. The method of any one of claims 83 to 103, comprising a first treatment cycle.

105. The method of claim 104, wherein the first treatment cycle comprises administration of lymphodepleting chemotherapy prior to administration of the NK cells.

106. The method of claim 105, wherein administration of lymphodepleting chemotherapy comprises administration of cyclophosphamide and fludarabine.

107. The method of claim 106, wherein fludarabine is administered on days 1, 2, and 3 of the treatment cycle.

108. The method of claim 106 or 107, wherein the fludarabine is administered at 30 mg/m²/day.

109. The method of any one of claims 106-108, wherein the cyclophosphamide is administered on day 3 of the treatment cycle.

110. The method of any one of claims 106-109, wherein the NK cells are administered at least 48 hours after the last administration of the lymphodepletion chemotherapy.

111. The method of any one of claims 106-110, wherein the cyclophosphamide is administered at 100 mg/m²/day.

112. The method of any one of claims 104-111, wherein the antibody is administered on days 2 and 13 of the treatment cycle.

113. The method of any one of claims 104-112, wherein the antibody is rituximab.

114. The method of claim 113, wherein the antibody is administered at 1000 mg.

115. The method of any one of claims 104-114, wherein the NK cells are administered on one or more of days 6, 9, 13, 16, and 20.

116. The method of claim 115, wherein the NK cells are administered on days 6, 13, and 20.

117. The method of claim 115, wherein the NK cells are administered on days 6, 9, 13, and 16.

118. The method of any one of claims 104-114, wherein the NK cells are administered at 2×10\hat{ }9 cells on days 6 and 13 and 1×10\hat{ }9 cells on day 20.

119. The method of any one of claims 104-114, wherein the NK cells are administered at 4×10\hat{ }9 cells on days 6 and 13, and 2×10\hat{ }9 cells on day 20.

120. The method of any one of claims 104-114, wherein the NK cells are administered at 4×10\hat{ }9 cells on days 6 and 13, and 2×10\hat{ }9 cells on days 9 and 16.

121. The method of any one of claims 104-120, further comprising a second treatment cycle.

122. The method of claim 121, wherein the second treatment cycle comprises administration of lymphodepleting chemotherapy prior to administration of the NK cells.

123. The method of claim 122, wherein administration of lymphodepleting chemotherapy comprises of the second treatment cycle comprises administration of cyclophosphamide and fludarabine.

124. The method of claim 123, wherein fludarabine is administered on days 1, 2, and 3 of the second treatment cycle.

125. The method of claim 123 or 124, wherein the fludarabine is administered at 30 mg/m²/day during the second treatment cycle.

126. The method of any one of claims 123-125, wherein the cyclophosphamide is administered on day 3 of the second treatment cycle.

127. The method of any one of claims 122-126, wherein the NK cells are administered at least 48 hours after the last administration of the lymphodepletion chemotherapy during the second treatment cycle.

128. The method of any one of claims 122-126, wherein the cyclophosphamide is administered at 100 mg/m²/day during the second treatment cycle.

129. The method of any one of claims 121-128, wherein the antibody is administered on days 2 and 13 of the second treatment cycle.

130. The method of any one of claims 121-129, wherein the antibody administered during the second treatment cycle is rituximab.

131. The method of claim 130, wherein the antibody is administered at 1000 mg during the second treatment cycle.

132. The method of any one of claims 121-131, wherein the NK cells are administered on one or more of days 6, 9, 13, 16, and 20 of the second treatment cycle.

133. The method of claim 132, wherein the NK cells are administered on days 6, 13, and 20 of the second treatment cycle.

134. The method of claim 132, wherein the NK cells are administered on days 6, 9, 13, and 16 of the second treatment cycle.

135. The method of any one of claims 121-132, wherein the NK cells are administered at 2×10\hat{ }9 cells on days 6 and 13 and 1×10\hat{ }9 cells on day 20 of the second treatment cycle.

136. The method of any one of claims 121-132, wherein the NK cells are administered at 4×10\hat{ }9 cells on days 6 and 13, and 2×10\hat{ }9 cells on day 20 of the second treatment cycle.

137. The method of any one of claims 121-132, wherein the NK cells are administered at 4×10\hat{ }9 cells on days 6 and 13, and 2×10\hat{ }9 cells on days 9 and 16 of the second treatment cycle.

138. The method of any one of claims 121-137, wherein the second treatment cycle is administered 18-30 weeks after the first dose of NK cells in the first treatment cycle.

139. The method of claim 138, wherein the second treatment cycle is administered about 24 weeks after the first dose of NK cells in the first treatment cycle.

140. The method of any one of claims 83-139, wherein the patient has lupus nephritis Class III or IV.

141. The method of claim 140, wherein the patient has lupus nephritis Class III.

142. The method of claim 140, wherein the patient has lupus nephritis class IV.

143. The method of any one of claims 84-142, wherein the patient has lupus nephritis Class V.

144. The method of any one of claims 83-143, wherein the patient who is relapsed or refractory after receiving a previous treatment for SLE.

145. The method of claim 144, wherein the previous treatment is selected from the group consisting of glucocorticoids in combination with either mycophenolate mofetil (MMF) or cyclophosphamide, MMF in combination with either a calcineurin inhibitor (e.g., voclosporin or tacrolimus) or belimumab, cyclophosphamide in combination with belimumab, intravenous cyclophosphamide, an anti-CD20 monoclonal antibody (mAb), or intravenous cyclophosphamide in combination with an anti-CD20 monoclonal antibody (mAb), or a combination thereof.

146. The method of any one of claims 83-145, wherein the patient's Overall Renal Response Rate (ORR) is improved after treatment.

147. The method of claim 146, wherein the improvement is Complete Renal Response (CRR).

148. The method of claim 147, wherein CRR is measured as a urinary protein-to-creatinine ratio (UPCR) of <0.5 and/or normal renal function without worsening of baseline serum creatinine by more than 15%.

149. The method of claim 148, wherein normal renal function is measured as serum creatinine less than or equal to ULN.

150. The method of claim 146, wherein the improvement is Partial Renal Response (PRR).

151. The method of claim 150, wherein PRR is measured as a 50% or more reduction in UPCR from baseline to a value<3.

152. The method of claim 151, wherein PRR is measured as a 50% or more reduction in UPCR from baseline to a value<1.

153. The method of any one of claims 146-152, wherein the ORR is measured within 1, 2, 3, 4, 5, 6, or 7 days after administration.

154. The method of any one of claims 146-152, wherein the ORR is measured within 1, 2, 3, 4, 5, 6, 7, 8, 9, 10 or 12 months after administration.

155. The method of any of the forgoing claims, wherein the NK cells are not genetically modified.

156. The method of any of the forgoing claims, wherein at least 70% of the NK cells are CD56+ and CD16+.

157. The method of any of the forgoing claims, wherein at least 85% of the NK cells are CD56+ and CD3−.

158. The method of any of the forgoing claims, wherein 1% or less of the NK cells are CD3+, 1% or less of the NK cells are CD19+ and 1% or less of the NK cells are CD14+.

159. The method of any of the forgoing claims, wherein the NK cells are expanded umbilical cord blood natural killer cells.

160. The method of any of the forgoing claims, wherein the population comprises at least 60%, e.g., at least 70%, at least 80%, at least 90% at least 95%, at least 99%, or 100% CD16+ cells.

161. The method of any of the forgoing claims, wherein the population comprises at least 60%, e.g., at least 70%, at least 80%, at least 90% at least 95%, at least 99%, or 100% NKG2D+ cells.

162. The method of any of the forgoing claims, wherein the population comprises at least 60%, e.g., at least 70%, at least 80%, at least 90% at least 95%, at least 99%, or 100% NKp46+ cells.

163. The method of any of the forgoing claims, wherein the population comprises at least 60%, e.g., at least 70%, at least 80%, at least 90% at least 95%, at least 99%, or 100% NKp30+ cells.

164. The method of any of the forgoing claims, wherein the population comprises at least 60%, e.g., at least 70%, at least 80%, at least 90% at least 95%, at least 99%, or 100% DNAM-1+ cells.

165. The method of any of the forgoing claims, wherein the population comprises at least 60%, e.g., at least 70%, at least 80%, at least 90% at least 95%, at least 99%, or 100% NKp44+ cells.

166. The method of any of the forgoing claims, wherein the population comprises less than 20%, e.g., 10% or less, 5% or less, 1% or less, 0.5% or less, or 0% CD3+ cells.

167. The method of any of the forgoing claims, wherein the population comprises less than 20% or less, e.g., 10% or less, 5% or less, 1% or less, 0.5% or less, or 0% CD14+ cells.

168. The method of any of the forgoing claims, wherein the population comprises less than 20% or less, e.g., 10% or less, 5% or less, 1% or less, 0.5% or less, or 0% CD19+ cells.

169. The method of any of the forgoing claims, wherein the population comprises less than 20% or less, e.g., 10% or less, 5% or less, 1% or less, 0.5% or less, or 0% CD38+ cells.

170. The method of any of the forgoing claims, wherein the natural killer cells do not comprise a CD16 transgene.

171. The method of any of the forgoing claims, wherein the natural killer cells do not express an exogenous CD16 protein.

172. The method of any of the forgoing claims, wherein the natural killer cells are not genetically engineered.

173. The method of any of the forgoing claims, wherein the natural killer cells are derived from the same umbilical cord blood donor.

174. The method of any of the forgoing claims, wherein the population of NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, I trillion, 2 trillion, 3 trillion, 4 trillion, 5 trillion, 6 trillion, 7 trillion, 8 trillion, 9 trillion, or 10 trillion expanded natural killer cells.

175. The method of any of the forgoing claims, wherein the population of NK cells is produced by a method comprising:
(a) obtaining seed cells comprising natural killer cells from umbilical cord blood:
(b) depleting the seed cells of CD3+ cells:
(c) expanding the natural killer cells by culturing the depleted seed cells with a first plurality of Hut78 cells engineered to express a membrane bound IL-21, a mutated TNFα, and a 4-1BBL gene to produce expanded natural killer cells,
thereby producing the population of expanded natural killer cells.

176. The method of any of the forgoing claims, wherein the population of NK cells is produced by a method comprising:
(a) obtaining seed cells comprising natural killer cells from umbilical cord blood;
(b) depleting the seed cells of CD3+ cells;
(c) expanding the natural killer cells by culturing the depleted seed cells with a first plurality of Hut78 cells engineered to express a membrane bound IL-21, a mutated TNFα, and a 4-1BBL gene to produce a master cell bank population of expanded natural killer cells; and
(d) expanding the master cell bank population of expanded natural killer cells by culturing with a second plurality of Hut78 cells engineered to express a membrane bound IL-21, a mutated TNFα, and a 4-1BBL gene to produce expanded natural killer cells;
thereby producing the population of expanded natural killer cells.

177. The method of claim 175 or claim 176, wherein the population of NK cells is produced by a method further comprising, after step (c),
(i) freezing the master cell bank population of expanded natural killer cells in a plurality of containers; and
(ii) thawing a container comprising an aliquot of the master cell bank population of expanded natural killer cells,
wherein expanding the master cell bank population of expanded natural killer cells in step (d) comprises expanding the aliquot of the master cell bank population of expanded natural killer cells.

178. The method of any one of claims 175 to 177, wherein the umbilical cord blood is from a donor with the KIR-B haplotype and homozygous for the CD16 158V polymorphism.

179. The method of any one of the forgoing claims, wherein the population of NK cells is produced by a method comprising expanding the natural killer cells from umbilical cord blood at least 10,000 fold, e.g., 15,000 fold, 20,000 fold, 25,000 fold, 30,000 fold, 35.000 fold, 40,000 fold, 45,000 fold, 50,000 fold, 55,000 fold, 60,000 fold, 65,000 fold, or 70,000 fold.

180. The method of any one of 175-179, wherein the population of expanded natural killer cells is not enriched or sorted after expansion.

181. The method of any one of the forgoing claims, wherein the percentage of NK cells expressing CD16 in the population is the same or higher than the percentage of natural killer cells in the seed cells from umbilical cord blood.

182. The method of any one of the forgoing claims, wherein the percentage of NK cells expressing NKG2D in the population is the same or higher than the percentage of natural killer cells in the seed cells from umbilical cord blood.

183. The method of any one of the forgoing claims, wherein the percentage of NK cells expressing NKp30 in the population is the same or higher than the percentage of natural killer cells in the seed cells from umbilical cord blood.

184. The method of any one of the forgoing claims, wherein the percentage of NK cells expressing NKp44 in the population is the same or higher than the percentage of natural killer cells in the seed cells from umbilical cord blood.

185. The method of any one of the forgoing claims, wherein the percentage of NK cells expressing NKp46 in the population is the same or higher than the percentage of natural killer cells in the seed cells from umbilical cord blood.

186. The method of any one of the forgoing claims, wherein the percentage of NK cells expressing DNAM-1 in the population is the same or higher than the percentage of natural killer cells in the seed cells from umbilical cord blood.
